(19) United States
(12) Patent Application Publication
(10) Pub. No.: US 2026/0229353 A1
(43) Pub. Date: Aug. 6, 2026

(54) METHOD OF PROFILING DISEASES

(71) Applicant: AKRIVIA BIOMEDICS LIMITED, London (GB)

(72) Inventor: Amit Gupta, London (GB)

(73) Assignee: AKRIVIA BIOMEDICS LIMITED, London (GB)

(21) Appl. No.: 19/147,888

(22) PCT Filed: Jan. 12, 2024
(86) PCT No.: PCT/GB2024/050085

§ 371 Date: Jul. 14, 2025

(30) Foreign Application Priority Data
Jan. 13, 2023 (EP) 23151631.1

(51) Int. Cl. G16H 50/20; G16B 20/20; G16B 25/10; G16B 40/20; G16H 10/40; G16H 10/60; G16H 50/30; G16H 50/70

(52) U.S. Cl. CPC G16H 50/20; G16B 20/20; G16B 25/10; G16B 40/20; G16H 10/40; G16H 10/60; G16H 50/30; G16H 50/70

(57) ABSTRACT

The present invention provides a method of stratifying genomic aberrations associated with a disease and subjects suffering from a disease comprising at least one of the same genomic aberrations. Also provided are methods of determining the prognosis of the subject as well as methods of determining a treatment plan for the subject. In addition, the invention provides computer programs and apparatus, such as a computer, for executing the methods of the invention. Further provided are methods of identifying biomarkers for stratified subjects and databases and/or models for stratifying subject using the methods of the invention.

### FIELD OF INVENTION

The present invention provides a method of stratifying genomic aberrations associated with a disease and subjects suffering from a disease comprising at least one of the same genomic aberrations. Also provided are methods of determining the prognosis of the subject as well as methods of determining treatment plan for the subject. In addition, the invention provides computer programs and apparatus, such as a computer, for executing the methods of the invention. Further provided are methods identifying biomarkers for stratified subjects and databases and/or models for stratifying subjects using the methods of the invention.

### BACKGROUND

Cancer ranks as a leading cause of morbidity and mortality worldwide, in every world region, irrespective of the level of human development. There have been an estimated 10 million cancer deaths worldwide in 2020 alone, with over 19.3 million new cancer cases. Moreover, the global cancer burden would reach 28.4 million cases by 2040, an almost 50% rise from 2020. Increases in mortality rates will likely parallel increases in cancer incidences unless resources are placed within health services to treat and manage the growing number of cancers appropriately. Thus, the provision of good cancer care is critical for global cancer control.

However, the current approach undertaken for cancer treatment can be best described as one-size-fits-all. Unfortunately, though, cancer drugs don't work the same way for everyone. A report published by Personalized Medicine Coalition in 2017 pointed out that any particular class of cancer drugs is ineffective in 75% of patients and unfortunately, it takes too long to figure this out until the disease relapses or gets worse. Due to the one-size-fits-all approach, a large number of cancer patients fail to respond to their prescribed treatments and experience serious adverse side effects, which drastically compromises their quality of life and even contributes to their premature death and shorter survival. This has a significant impact on society and the economy. The economic burden due to adverse drug reactions is more than 30 billion dollars annually in the United States alone. In addition, most cancer drugs are expensive, and it is thus difficult to justify widespread usage when it is not possible to predict if a subject will respond well to a drug. Currently, there are no clinically useful tests available to predict a response to treatments and guide the selection of the most appropriate therapies for individual patients.

Moreover, the detection of diseases such as cancer at early stages with current screening programmes has led to a major problem of cancer overdiagnosis and overtreatment. While an early and accurate diagnosis of diseases such as cancer can lead to improved quality of life and better treatment modalities, over-diagnosis can lead to unnecessary and costly interventions that cause undesirable morbidities. In order to be able to anticipate the natural history of the disease in individual patients and avoid over treatment, the nature of the disease in each subject should be clearly understood since only individually tailored molecular profiles and markers could spare the patients from undergoing a potentially more harmful, aggressive chemical therapy or even leave them untreated.

In today's era of targeted therapy (the latest generations of anticancer agents founded on biological mechanisms), it is essential to identify subject subsets that are either likely or unlikely to respond to a particular drug. Doing so would maximise the efficacy and minimise unnecessary toxicity associated with the use of the drug in question. More than 850 molecularly targeted cancer drugs are currently in development or clinically available. Finding the right target subject population is the key to the commercial success of each. Also, retargeting or repurposing existing drugs to new populations could unlock substantial untapped revenues for pharma and help patients, and providers. Thus, it is more important than ever to develop diagnostic tests that can guide selecting the most appropriate therapies for individual patients.

Precision medicine is an emerging approach to medical treatment that allows doctors to select treatment regimens that are most likely to help patients based on the genetic understanding of their disease. For cancer therapy, however, there are significant challenges in applying personalised and precision medicine approaches. Cancer is a highly complex, extremely heterogeneous disease with a highly chaotic molecular landscape. It is a disease of genome with high levels of genomic aberrations present within a patient's cancerous growth. Typically, a single tumour contains anywhere from tens to millions of genomic aberrations-including point mutations, gene fusions, small insertions, small deletions, and large copy number alterations (CNAs)—that differ from the patient's normal cells. Also, genomic aberrations within tumours are highly variable across cancer patients—each tumour carries a unique panoply of genomic aberrations with no two tumours genomically the same. This intrinsic complexity of cancer is not associated with complete knowledge of its biology, and even less with the availability of markers for use in clinical practice which fully reflect its genomic and biological complexity, and which allow an accurate prognosis to be made in each case and enable the likelihood of success of the treatments to be determined. These factors are essential for guiding the choice of treatment and, when necessary, the development of new intervention strategies. The genetic complexity represents a major impediment to designing effective personalised treatment strategies against cancer.

The classification of tumours based on morphology (histological type and grade) remains the mainstay of current clinical practice. Early attempts to improve this situation by using genomic technology focused on data-driven methods, including unsupervised transcriptome-based classification (PAM50 classification method for breast cancer & others) and gene signatures trained against a specific clinical outcome. However, this approach is not based on the underlying molecular changes which ultimately constitute a tumour's oncogenic drive. As a result, they fail to capture the full complexity of tumoural heterogeneity and are thus not suitable for precision medicine purposes. Moreover, it is often argued that first-generation prognostic signatures only constitute a reproducible and quantitative analysis of tumour cell proliferation. Their prognostic power is primarily related to assessing proliferation-related/cell-cycle-related genes. Rightly, some investigators have argued that these first-generation signatures are nothing but mere surrogates of proliferation. Furthermore, it has become clear that the first-generation prognostic gene signatures provide complementary information and not a replacement for that provided by current prognostic algorithms based on clinicopathologic features. There remains a need for better prognostic or predictive biomarkers for cancers.

Over the past decade, the development of high-throughput technologies to study genomic, transcriptomic, epigenetic and proteomic changes have allowed for rapid progress in the understanding of the complexity of disease biology, such as cancer biology. There have been recent attempts to integrate this molecular information, for example, by classifying tumours based on unsupervised analysis of paired CNA-transcriptome profiles (including IntClust as a genomic and transcriptome integrated classifier for breast cancer by Carlos Caldas' group). However, stratification of cancers based on such integrative approaches have not been able to refine the molecular classification of the disease so far. Often these computer model-based integrative classifications systems have limited clinical utility and are too complicated, and it is a challenge to make sense from the information they provide. The biology of disease is much more complex. For example, for cancer it involves the tumour microenvironment and crosstalk between various signalling pathways, including the immune system. Many of these first and second-generation multi-gene prognostic signatures, thus, do not capture the true complexities of the tumour.

Over the last decade, predictive biomarkers, primarily based on single genes, have also been approved with their companion therapeutic agents (for instance, PIK3CA mutation has been approved as a predictive biomarker for treatment response to PI3Kα inhibitor, PIQRAY, which is a highly toxic drug). This ‘single-gene biomarker’ precision medicine strategy focuses on single genetic alterations/mutations without understanding its nuance, context and importance. The approach only works in a small proportion of cancers; up to 90% of cancer patients become resistant to targeted drugs and suffer unnecessary side effects. Given the highly heterogeneous and chaotic landscape in cancer biology, the isolated molecular entities have little/no value on their own for precision medicine purposes. Advances in DNA sequencing technologies indicate that the average subject with site-specific solid tumours such as lung cancer have 200-300 non-synonymous mutations per tumour in a single patient, while patients with breast, oesophageal or colon cancer have somewhere between 50 to 500 mutations per tumour. Consequently, making decisions on such evolving high rates of mutations in solid human tumours make these approaches fraudulent (‘molecular false flags’) and irresponsible, as evident from the high failure rate outcomes of ‘molecular target’ therapies. The claimed molecular target drugs that aim at one or two specific mutations of growth factors, receptors, or enzymes, whether or not the mutations are at the “driver seat” at the time they are identified, would maximally have 1-3% chances of therapeutic success.

A primary aim in cancer management is to tailor clinical decisions to the individual, based on a detailed understanding of the molecular profile of the tumour and the likely clinical outcome of the individual's disease. This progress will facilitate personalised treatment approaches that are more targeted, have superior efficacy and are associated with less toxicity. Moreover, because the latest generations of targeted therapies are founded on biological mechanisms, a detailed molecular stratification is a requirement for appropriate clinical management. Such stratification, based on molecular drivers, will be important for selecting patients for clinical trials in which response to therapy is evaluated. It will also facilitate the discovery of novel drivers, the study of tumour evolution, the identification of mechanisms of treatment resistance, and to inform combination therapy strategies.

An increased understanding of the genomic complexity of a disease is the key to achieving the aims of personalised medicine. However, the genomic complexity of diseases such as cancer is likely far more complex than the current understanding of multi-dimensional or the existing molecular classifications. What is needed are molecular classification systems that take into careful account the genomic complexity of a disease and its immune landscape and provide actionable insights into the underlying biology driving the disease in individual patients.

### BRIEF SUMMARY OF THE DISCLOSURE

The present invention relates to methods for classifying genomic aberrations associated with a disease and subjects suffering from a disease that includes at least one of the same genomic aberrations into biologically informative molecular subgroups, evaluating prognosis and strategies of identifying a subject with the disease as a candidate for a therapy having a specific efficacy for treating a molecular subgroup, and selecting a treatment for a subject with the disease. For example, the disease may be cancer.

The methods include determining molecular subgroups of a disease and assigning subjects suffering from the disease to one of the molecular subgroups for predicting and informing possible response to specific therapies in subjects with the disease in question and administering specific therapies that are effective for treating the molecular subgroup. The methods provide biological insights into the potential molecular drivers and pathways underlying the molecular subgroups, with distinct implications for the rational development of targeted therapeutics. The methods also may help identify subject subsets who would not require any therapeutic interventions and thus could be prevented from being overtreated with harmful interventions. The invention further includes methods for identifying novel targets and biological pathways (biomarkers) for therapeutic interventions for a molecular cancer subgroup. The methods further comprise identifying and stratifying subject subsets to optimise subject enrolment onto clinical trials testing the efficacy of various investigational and marketed/approved therapeutics. The methods may also provide a more accurate prognosis for a subject that has a disease such as cancer. Being able to consider the biological, molecular and immunological context of the disease, the methods described herein include informing the rational for therapy strategies to help provide improved responses.

The invention encompasses a multi-tier approach that integrates the diseases genomic and transcriptomic profiles with the information on the diseases immune landscape. The invention helps to identify subjects with active immune systems from specific molecular subgroups of disease that would benefit from for example immune checkpoint inhibitor therapies and/or wherein immediate therapeutic intervention may not be required, and the subject may be selected for active surveillance based on their disease prognosis without any therapeutic intervention.

Using cancer as an example, the invention classifies genomic aberrations present in control subjects suffering from the cancer into two groups (Group A and Group B) based on genomic aberrations downstream transcriptional effects [the fold-change direction of differentially expressed genes (DEGs)]. The fold-change direction of DEGs between the Group A and Group B genomic aberrations, in general, follow an inverse expression pattern. The Group A set of genomic aberrations may include TP53 gene mutations, the majority of the somatic copy number alterations (SCNAs) and gene fusions. Group A also has any other genomic aberrations/somatic mutations that follow similar transcriptome expression patterns (the fold-change direction of DEGs) to a control gene such as TP53 gene mutations. In contrast, Group B includes genomic aberrations that broadly follow the fold-change direction of the shared (overlapping) DEGs contrariwise to Group A genomic aberrations. The Group B set of genomic aberrations include, for example, PIK3CA, MAP3K1, GATA3, CDH1, and KMT2C gene mutations in breast cancer; PTEN and PIK3CA gene mutations in endometrial cancers; and, SPOP and FOXA1 gene mutations in prostate cancers.

The invention selects the shared (overlapping) set of DEGs between these two groups (Groups A and B). Tier 1 classification is based on the gene expression profiles of the shared (overlapping) set of genes between Group A and Group B genomic aberrations for the cancer. Using gene set enrichment analysis (GSEA) it can be seen that tumours with genomic aberrations in Group B may in a majority of cases have gene signatures related to critical tumour growth-supporting pathways downregulated. This includes cell cycle, DNA replication, RNA transport and ribosome-biogenesis-associated molecular pathways. Given this the cancer type-specific shared (overlapping) set of DEGs are altered with most cancer-related genomic alterations and generally follow an inverse expression change trend with the genomic aberrations of Group B associated with the downregulation of cancer-promoting pathways' signature genes, this highlights their utility in defining the aggregate effects of driver genomic alterations in cancer. This ‘global’ feature provides an exceptional opportunity to comprehend the genomic complexities of cancer and understand the nuance, context and importance of the complex genomic alterations and thereby predict the best clinical and therapeutic approach for cancer patients. The present invention exploits the advantages provided by this ‘global’ feature to delineate the genomic and molecular complexities associated with various cancer types.

The invention herein describes a novel application of these findings in disease diagnostics and prognosis and precision medicine. The invention describes an application of these findings in classifying, for example various cancer types into different biologically informative molecular subgroups, utilising a multi-tier approach. Firstly, the method involves tentatively grouping a sample from a cancer type into a low or high-risk group based on a genomic score (also referred to as a risk score or AG score) derived from a two or more DEGs (disease-specific gene signature selected from the shared DEG sets described above).

A close examination of this ‘global’ feature across various cancer types reveals that genomic aberrations in Group A transcriptionally follow similar cancer development mechanisms broadly. Genomic aberrations within Group A can have distinct impacts on AG scores but usually have a linear relation to prognosis. The higher the impact/contribution of a genomic aberration on the AG-score, the worse the prognosis, or the lower the impact/contribution of the genomic aberration on the AG-score, the better the prognosis. In most cancer types, usually, the TP53 mutations have the highest impact on the AG scores among the Group A genomic aberrations. However, each genomic aberration in Group B tends to follow a somewhat unique mechanism of cancer development transcriptionally. The genomic aberrations in Group B across the cancer types usually contribute to lower-AG scores. However, irrespective of the contribution of genomic aberrations in Group B to the AG-score, it does not always follow a linear relation with the overall prognosis. That means a Group B genomic aberration resulting in a relatively lower AG score can have a worse prognosis than a genomic aberration resulting in a considerably higher AG score and vice versa.

The second-tier classification involves further subgrouping the high and low-risk groups based on the cancer type-specific sample genomic profile.

The third tier of molecular classification includes subgrouping based on an immune gene signature reflecting the tumour's immune landscape.

The fourth tier involves classifying the above-identified groups into additional subclasses based on the median AG score (genomic score/risk score).

The fifth tier involves classifying these sub-groups into further subclasses based on the mutational profiles of genes not included in the tier-two classification level.

The sixth tier of molecular classification includes subgrouping based on a metastatic gene signature reflecting the tumour's metastatic potential.

It will also be understood that each tier of classification may be used independently of each other tier or in any combination. In particular, for example, the methods may include carrying out one of Tier 1, Tier 3, or Tier 6 classification. The methods may include at least one of Tier 1, Tier 3, or Tier 6 classification and further include tier 2, tier 4 and/or tier 5 classification.

This first-of-its-kind multi-tier cancer classification approach helps refine the biology and prognosis at each tier by separating tumours into different subgroups. This proactive, holistic multi-tier classification method enables dissection of a diseases biology and prognosis in detail.

The method also allows for the identification of group-specific biomarkers in each group of each tier. This set of biomarkers provides a signature that can be used to determine which group a test subject may fall within without the need for carrying out the complete methods described herein. For example, a group-specific biomarker or biomarkers can be used as part of tests in the traditional histopathological clinical setting to stratify or classify subjects solely based on the levels of the group-specific biomarkers in a subject sample.

In the case of cancer, this multi-tier analysis will classify samples, such as tumour samples in various sub-groups that have specific tumour biology's and clinical courses, which may directly affect subject treatment recommendations. This integrated multi-tier analysis provides key molecular insights into disease (e.g. tumour) biology, which may directly affect treatment recommendations for patients. In addition, it provides opportunities for precision medicine, biomarker-guided clinical trials and the development of novel drugs. Other innovative features associated with our molecular classifiers include:
a) Identifying subject subsets that would not require any therapeutic interventions and thus could be prevented from being over treated with harmful interventions.
b) Predicting a more accurate disease prognosis/clinical outcomes compared with existing classification schemes.

In one aspect of the invention there is provided a method of classifying one or more genomic aberrations associated with a disease, the method comprising: identifying genes in a plurality of control subjects suffering from the disease that undergo a change of expression in response to a first genomic aberration and selecting the genes that undergo a change of expression in response to the first genomic aberration to provide a first set of differentially expressed genes (DEGs) associated with the first genomic aberration; identifying DEGs of the first set of DEGs that overlap with DEGs of a control set of DEGs for a control genomic aberration and selecting the overlapping DEGs to form a first set of overlapping DEGs; comparing the fold direction of change of expression of each DEG of the first set of overlapping DEGs to a fold direction change of expression of the corresponding DEG of the control set of DEGs; classifying the first genomic aberration into a first or second group wherein: the first group comprises at least 51% overlapping DEGs that comprises a fold direction of change of expression that is the same as the fold direction of change of expression as the corresponding DEG of the control genomic aberration (Group A); the second group comprises at least 51% overlapping DEGs that comprise a fold direction of change of expression that is inverse to the fold direction of change of expression of the corresponding DEG of the control genomic aberration (Group B). In certain embodiments, the method comprises stratifying a subject suffering from the disease, wherein stratifying comprises; calculating a risk score for the subject based on the classified genomic aberration and the subject is stratified based on the risk score; and wherein the calculated risk score is indicative of prognosis of the subject.

In another aspect of the invention there is provided a method of calculating an immune risk score for a subject suffering from a disease, the disease comprising one or more genomic aberrations, the method comprising: selecting two or more immune associated genes associated with disease to form at least one immune signature; optionally performing further statistical analysis such as Lasso, univariate, and/or multivariate Cox regression analyses to identify prognosis-related immune associated genes for a immune signature (disease-specific immune signature); assigning a direction of association to each gene of the immune signature based on a change of expression for a plurality of control subjects; wherein each gene with an increased expression level is designated with a direction of association and each gene with a decreased expression is designated with a second direction of association inverse to the first direction of association; determining an expression level for each gene of the immune signature based on a level of RNA transcript for each gene for the plurality of control subjects; and determining an expression level for each gene of the immune signature based on a level of RNA transcript for each gene for the subject. In certain embodiments, the method comprises stratifying the subject wherein stratifying comprises, calculating an immune risk score based on the direction of association and the expression level of the each gene of the immune signature of the subject stratifying the subject as high immune risk, a low immune risk and/or an intermediate immune risk based on the calculated immune risk score; and wherein the immune risk score is indicative of a prognosis of the subject.

In another aspect of the invention there is provided a method of calculating a metastatic score for a subject suffering from a disease, the disease comprising one or more genomic aberrations, the method comprising: selecting two or more metastatic/dissemination associated genes associated with the disease to form at least one metastatic signature; optionally performing statistical analysis such as Lasso, univariate, and/or multivariate Cox regression analyses to identify prognosis-related metastatic/dissemination associated genes for the disease-specific metastatic signature; assigning a direction of association to each gene of the disease-specific metastatic signature based on a change of expression for a plurality of control subjects; wherein each gene with an increased expression level is designated with a direction of association and each gene with a decreased expression is designated with a second direction of association inverse to the first direction of association; determining an expression level for each gene of the disease-specific metastatic signature based on a level of RNA transcript for each gene for the plurality of control subjects; and determining an expression level for each gene of the disease-specific metastatic signature based on a level of RNA transcript for each gene for the subject. In certain embodiments, the method further comprises stratifying the subject wherein stratifying comprises, calculating a metastatic risk score based on the direction of association and the expression level of the each gene of disease-specific metastatic signature of the subject stratifying the subject as high metastatic risk, a low metastatic risk or an intermediate metastatic risk based on the calculated metastatic risk score; and wherein the metastatic risk score is indicative of a prognosis of the subject.

In another aspect of the invention there is provided a method of determining a prognosis for a subject suffering from a disease, the disease comprising one or more genomic aberrations, the method comprising: providing a subject sample; identifying one or more genomic aberrations associated with the disease from the subject sample; classifying the one or more genomic aberrations and stratifying the subject as described herein; and determining a prognosis for the subject based on the risk score. In certain embodiments, method may further comprises further stratifying the subject based on analysis according to any one or more of tiers 2 to 6 as described herein.

In another aspect of the invention there is provided a method of determining a prognosis for a subject suffering from a disease, the disease comprising one or more genomic aberrations, the method comprising: providing a subject sample; analysing the subject sample and calculating an immune risk score for the subject and stratifying the subject as described herein; and determining a prognosis for the subject based on the immune risk score. In certain embodiments, method may further comprises further stratifying the subject based on analysis according to any one or more of tiers 1 2, 4 and/or 6 as described herein.

In another aspect of the invention there is provided a method of determining a prognosis for a subject suffering from a disease, the disease comprising one or more genomic aberrations, the method comprising: providing a subject sample; analysing the subject sample and calculating a metastatic risk score for the subject and stratifying the subject according as described herein; and determining a prognosis for the subject based on the metastatic risk score. In certain embodiments, method may further comprises further stratifying the subject based on analysis according to any one or more of tiers 1 2, 4 and/or 5 as described herein.

In another aspect of the invention there is provided a treatment for cancer for use in a method of treating a subject suffering from a cancer comprising one or more genomic aberrations, wherein the subject has been stratified as described herein. In another aspect of the invention there is provided use of the methods as described herein as a companion diagnostic.

In another aspect of the invention there is provided a computer program comprising instructions which, when the program is executed by a computer, cause the computer to carry out at least one of the methods described herein. In another aspect of the invention there is provided a computer-implemented method for generating a classification model for classifying genomic aberrations to stratify patients into one or more groups.

In another aspect of the invention there is provided a method for predicting a prognosis of a subject suffering from a disease comprising; determining one or more group-specific biomarkers as described herein; measuring a level of one or more of the group-specific biomarkers in a sample obtained from the subject; classifying the subject into one or more of the Tier 1, Tier 2, Tier 3, Tier 4, Tier 5 and/or Tier 6 groups based on the level of the one or more of group-specific biomarkers; and predicting the prognosis of the subject based on the classification.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, example or example of the invention are to be understood to be applicable to any other aspect, or example described herein unless incompatible therewith.

Various aspects of the invention are described in further detail below.

### BRIEF DESCRIPTION OF THE FIGURES

Examples of the invention are further described hereinafter with reference to the accompanying drawings, in which:

FIG. 1 show that Group A and Group B genomic aberrations follow inverse (contrariwise) downstream transcriptional effects [the fold-change direction of significant differentially expressed genes (DEGs) in breast cancer: (A) Log fold change (LogFC) of shared (overlapping) DEGs from top 200 statistically significant genes between the following DEG lists: (a) TP53-Mutant (Group A genomic aberration) relative to TP53 wild-type (VVT) tumour samples (TP53-MUT), and (b) PIK3CA-Mutant (Group B genomic aberration) relative to PIK3CA-VVT tumour samples (PIK3CA-MUT). (B) LogFC analysis of DEGs from FIG. 1A (between top 200 DEGs from TP53-MUT and PIK3CA-MUT DEG lists) in the following statistically significant DEG lists: (a) RB1-Mutant (Group A genomic aberration) relative to RB1-WT tumour samples (RB1-MUT), (b) MAP3K1-Mutant (Group B genomic aberration) relative to MAP3K1-VVT tumour samples (MAP3K1-MUT), and (c) GATA3-Mutant (Group B genomic aberration) relative to GATA3-VVT tumour samples (GATA3-MUT). Shown are the shared (overlapping) DEGs. (C-D) mRNA expression analysis of a few representative shared (overlapping) DEGs from FIGS. 1A and 1B between Group A and Group B genomic aberrations. Log 2 mRNA expression analysis of (C) Nostrin RNA transcript and (D) CDCA5 RNA transcript. DEGs were considered significant using a threshold of FDR<0.05.

FIG. 2 shows examples of Tier-1 classification using several breast cancer datasets having risk scores derived from different gene signatures (for example, using two, three, four, five, six or eight combined groups of RNA transcripts/genes from the shared set of DEGs between the representative Group A and Group B genomic aberrations) to stratify breast cancer patients having used different risk score cut-off values for stratification: Kaplan-Meier plot showing breast cancer (BCa)-specific overall survival of patients in the first ten years after diagnosis in the GSE7390 (A), GSE1456 (B), GSE9195 (C-D) and METABRIC (E-G) breast cancer datasets belonging to different settings (e.g., having data from all patients or ER+ patients only wherein in some datasets patients were untreated with any systemic therapies or treated, for example, with hormone therapy as specified in the drawings). (A) The survival was analyzed according to the 4-genes (AURKA, ABAT, SLC7A5 and UBE2C) risk score (AG-score). An AG score cut-off of 13 is used to classify a subject as low (Low AG-Score) or high-risk (High AG-Score). (B) 4-genes (ABAT1, HSP90AA1, Nostrin and SLC7A5) risk score with a cut-off value of 17 used to classify a subject as low (Low AG-Score) or high-risk (High AG-Score). (C-D) 8-genes (CAV1, GCH1, LRP8, ABAT1, Nostrin, AURKA, UBE2C and SLC7A5) risk score with a cut-off value of 33 used to classify a subject as low (Low AG-Score) or high-risk (High AG-Score) in selected settings—in all ER+ patients (C) or lymph node-positive ER+ patients only (D). (E-G) 4-genes (AURKA, Nostrin, UBE2C and CBX2) risk score with two cut-off values (10 and 12) are used to classify a subject as low (Low AG-Score) or high-risk (High AG-Score) in selected settings—in all untreated (patients did not receive adjuvant systemic therapies, i.e., chemotherapy and/or hormone therapy) patients (E) or ER+ untreated patients only (F) or in systemic therapy-untreated but radiotherapy treated and/or untreated breast cancer patients. The hazard ratio (HR) and confidence interval (CI) and p-value for comparisons high vs. low AG-score are shown in the Kaplan-Meier survival curves (Log-rank Test, GraphPad Prism). The number of patients (n) is shown in brackets.

FIG. 3 shows examples how Tier-2 classification approach is exploited to find the nuance, context and significance of PIK3CA gene mutations in breast cancer (the most recurrent alterations in breast cancer), whose prognostic and predictive values are still not well understood. (A-C and E-H) Kaplan-Meier plot showing breast cancer (BCa)-specific overall survival of breast patients in the first ten years after diagnosis in the METABRIC (A, B) and TCGA Firehose (C) breast cancer datasets. Patients belong to different settings (e.g., having data from all patients or ER+ patients only wherein in some datasets patients were untreated with any systemic therapies or treated, for example, with hormone therapy and/or chemotherapy wherein in some examples patients are lymph node-negative or -positive as specified in the drawings). The high and low-risk groups from Tier-1 classification are further sub-grouped based on the PIK3CA gene mutation profile for the Tier-2 classification. Also shown in (B) are the Tier-2 subgroups-specific average and median AG-scores. A threshold of p<0.05 is used to determine the statistical significance. (D) Log 2 mRNA levels of LATS2 RNA transcripts showing significantly higher expression in PIK3CA mutant tumours associated with a good clinical outcome (i.e., Low AG-score_PIK3CA-MUT) than the Low AG-score_PIK3CA-WT or High AG-score_PIK3CA-MUT [i.e. Subgroup 3 (with PIK3CA-MUT, TP53-WT genomic profile) and Subgroup 5 (with PIK3CA-MUT, TP53-MUT genomic profile). LATS2 transcripts were considered significant using a threshold of p<0.05. In (A-C and E-H) the survival was analyzed according to the 4-genes (AURKA, ABAT, SLC7A5 and UBE2C) risk score (AG-score). An AG score cut-off of 12 is used to classify a subject as low (Low AG-Score) or high-risk (High AG-Score). The hazard ratio (HR) and confidence interval (CI) and p-value for comparisons between various Tier-2 subgroups are shown in the Kaplan-Meier survival curves (Log-rank Test, GraphPad Prism). The number of patients (n) is shown in brackets.

FIG. 4 shows another way for Tier 2 classification based on the breast cancer-specific Group A (TP53) and Group B (PIK3CA) genomic aberrations where the high-risk group (High AG-score groups—from tier-1 classification step) were further subclassified into four subgroups with the following genomic profiles—Subgroup 2 (TP53-WT, PIK3CA-WT), Subgroup 3 (TP53-WT, PIK3CA-MUT), Subgroup 4 (TP53-MUT, PIK3CA-WT) and Subgroup 5 (TP53-MUT, PIK3CA-MUT). (A-E) Kaplan-Meier plot showing breast cancer (BCa)-specific overall survival of breast patients in the first ten years after diagnosis in the METABRIC breast cancer dataset. Patients in the examples provided belong to different settings (e.g., having data from all patients or ER+ patients only wherein in some examples patients were untreated with any systemic therapies or treated, for example, with hormone therapy and/or chemotherapy wherein in some examples patients are lymph node-negative or -positive as specified in the drawings). Also shown in (B) are the Tier-2 subgroups-specific average and median AG-scores. A threshold of p<0.05 is used to determine the statistical significance. (E) shows Subgroup 3 (TP53-WT, PIK3CA-MUT) breast cancer patients-specific improvement in overall survival with hormone therapy. In (A-E) the survival was analyzed according to the 4-genes (AURKA, ABAT, SLC7A5 and UBE2C) risk score (AG-score). An AG score cut-off of 12 is used to classify a subject as low (Low AG-Score) or high-risk (High AG-Score). The hazard ratio (HR) and confidence interval (CI) and p-value for comparisons between various Tier-2 subgroups are shown in the Kaplan-Meier survival curves (Log-rank Test, GraphPad Prism). The number of patients (n) is shown in brackets.

FIG. 5 shows identified biomarker(s) for the Tier-2 subgroups (A-E). Tier-2 subgroups-specific tumour biology (pathway/biomarker analysis at protein level) based on Reverse Phase Protein Array (RPPA) data from TCGA Firehose legacy breast cancer dataset in following settings: (A) all patients i.e. both ER+ and ER− breast cancer cohort, (B) NO nodal stage (lymph node-negative) patients, (C) N1 and above nodal stage (lymph node-positive) patients, (D) in N2 and N3 nodal stage (with >3 lymph node involvement) patients only, and (E) in breast patients with PIK3CA hotspot H1047 kinase domain mutation. Following breast cancer patients were excluded from the analysis in (A-E): patients with Gata3, MAP3K1 and/or PTEN mutant profiles and HER2+ (with 3+ IHC score) patients. A threshold of p<0.05 is used to determine the statistical significance between the Tier-2 subgroups.

FIG. 6 shows examples of Tier-3 classification, which includes subgrouping based on an immune gene signature reflecting the tumour's immune landscape. The provided examples encompass various breast cancer histopathological subtypes and stratifies patients using different immune risk score cut-off values as Low-immune risk score and High-immune risk score subgroups. Kaplan-Meier plot showing breast cancer (BCa)-specific overall survival of breast patients in (A) systemically untreated [patients did not receive adjuvant systemic therapies, i.e., chemotherapy (C) and/or hormone therapy (H), but were treated or untreated with radiotherapy (R)]lymph node-negative ER+ high-risk (High AG-score) Subgroups 3, 4 and 5 breast cancer cluster, (B) systemically untreated lymph node-negative ER-negative HER2-negative (TNBC) high-risk (High AG-score) Subgroups 2, 3, 4 and 5 breast cancer cluster, (C) lymph node-positive TNBC high-risk Subgroups 2, 3, 4 and 5 breast cancer cluster, (E) lymph node-negative ER+ high-risk (High AG-score) Subgroups 3, 4 and 5 breast cancer cluster to identify cohort that benefits from hormone therapy. (D) Mean expression values of various immune checkpoints between Low AG-immune risk score vs. High AG-immune risk score patients. A threshold of p<0.05 is used to determine the statistical significance. Tier-3 classification shown in (A-E) are using METABRIC breast cancer dataset. Immune risk score cut-off of 30 and/or 27 (for drawing B) are used to classify a subject as Low-immune risk score or High-immune score. The immune risk score (AG-immune score) in (A-E) is derived using the following immune-associated genes from Table 5: CCL5, CD3D, CXCL9, CXCL10, GBP1, GZMB, and IDO1. In (A-E) the survival was analyzed according to the 4-genes (AURKA, ABAT, SLC7A5 and UBE2C) risk score (AG-score). An AG score cut-off of 12 is used to classify a subject as low (Low AG-Score) or high-risk (High AG-Score). The hazard ratio (HR) and confidence interval (CI) and p-value for comparisons between various Tier-3 subgroups are shown in the Kaplan-Meier survival curves (Log-rank Test, GraphPad Prism). The number of patients (n) in each subgroup is shown in drawings.

FIG. 7 shows examples of Tier-4 classification, which involves classifying the subgroups from tier 1, tier 2 or tier 3 classification into additional subclasses based on each subgroup's median AG-score (genomic score). (A) Kaplan-Meier plot showing breast cancer (BCa)-specific overall survival of breast patients in early-stage ER-negative high-risk subgroup cluster (identified from tier 3 classification, comprising Low_immune_score_Subgroups 2, 3, 4 and 5), this further classification step results in the identification of intermediate-risk cancer subgroups (those on the underside of each subgroup's median AG-score—Subgroups 2A, 3A, 4A and 5A) along with high-risk subgroups (those on upper-side of each subgroup's median AG-score—Subgroups 2B, 3B, 4B and 5B) that are characterised by relatively better and extremely poor prognosis, respectively. The hazard ratio (HR) and confidence interval (CI) and p-value for comparisons between Tier-4 subgroups are shown in the Kaplan-Meier survival curves (Log-rank Test, GraphPad Prism). The number of patients (n) in each subgroup is shown in drawings. (B-E) Identified biomarkers for the Tier-4 subgroups. Tier-4 subgroups-specific tumour biology (pathway/biomarker analysis at protein level) based on Reverse Phase Protein Array (RPPA) data (B, C) and mRNA expression data showing individual transcripts (D, E) from TCGA Firehose legacy breast cancer dataset in following settings: (B and D) ER+ breast cancer cohort, (C and E) ER− breast cancer cohort. Following breast cancer patients were excluded from the analysis in (A-E): patients with Gata3, MAP3K1 and/or PTEN mutant profiles and HER2+(with 3+ IHC score) patients. A threshold of p<0.05 is used to determine the statistical significance between the Tier-2 subgroups.

FIG. 8 shows examples of Tier-5 classification, which involves classifying the subgroups from tier 1, tier 2, tier 3 or tier 4 classification steps into further subgroups based on the mutational profiles of genes (from Group A and Group B genomic aberrations list—Table 1) not already directly included in the tier 2 classification. (A-D) shows sub-classifying high-risk and low-risk groups (Tier-2 subgroups) into further subgroups based on the tumour's CDH1 (A, B) or MAP3K1 (C, D) gene mutation statuses in the following settings: (A) all ER+ Subgroup 1 (Low-risk/Low AG-score) breast cancer patients, (B) Lymph-node positive ER+ Subgroup 1 breast cancer cohort, (C) systemically untreated [patients did not receive adjuvant systemic therapies, i.e., chemotherapy (C) and/or hormone therapy (H), but were treated or untreated with radiotherapy (R)]lymph node-negative ER+ breast cancer cohort, and (D) all ER+ lymph-negative breast cancer cohort. In (A-D) the Kaplan-Meier plot show breast cancer (BCa)-specific overall survival of breast patients. The survival was analyzed according to the 4-genes (AURKA, ABAT, SLC7A5 and UBE2C) risk score (AG-score). An AG score cut-off of 12 is used to classify a subject as low (Low AG-Score) or high-risk (High AG-Score). The hazard ratio (HR) and confidence interval (CI) and p-value for comparisons between various Tier-5 subgroups are shown in the Kaplan-Meier survival curves (Log-rank Test, GraphPad Prism). The number of patients (n) in each subgroup is shown in drawings. (E) Tier-5 subgroups (based on the tumour's CDH1 gene mutation status) tumour biology using Reverse Phase Protein Array (RPPA) in TCGA firehose ER+ breast cancer cohort. A threshold of p<0.05 is used to determine the statistical significance between the Tier-5 subgroups.

FIG. 9 shows classification based on the metastatic score (tier-6) derived from the metastatic gene signature (selected from the gene list provided in Table 8), further stratifies the Tier 1 (A) low-risk group (B) high-risk group into further prognostic molecular subgroups in lymph node-negative ER+ HER2− breast cancer patients. Patients were stratified based on the median metastatic score cut-off.

FIG. 10 shows that the multi-tier classification method used as described herein can be applied to other diseases and other cancer types. (A, B) shows that Group A and Group B genomic aberrations follow inverse (contrariwise) downstream transcriptional effects [the fold-change direction of significant differentially expressed genes (DEGs) in endometrial cancer and prostate cancer (B): (A) Log fold change (LogFC) of shared (overlapping) DEGs from top 200 statistically significant genes between the following DEG lists: (a) TP53-Mutant (Group A genomic aberration) relative to TP53 wild-type (VVT) tumour samples (TP53-MUT), (b) PTEN-Mutant (Group B genomic aberration) relative to PTEN-WT tumour samples (PTEN-MUT), and (c) (b) KRAS-Mutant (Group B genomic aberration) relative to KRAS-WT tumour samples (KRAS-MUT). (B) LogFC of shared (overlapping) DEGs from top 200 statistically significant genes between the following DEG lists: (a) TP53-Mutant (Group A genomic aberration) relative to TP53 wild-type (VVT) tumour samples (TP53-MUT), (b) SPOP-Mutant (Group B genomic aberration) relative to SPOP-WT tumour samples (SPOP-MUT), and (c) (b) TMPRSS2-ERG gene fusion (Group A genomic aberration) relative to VVT tumour samples (TMPRSS2-ERG gene fusion). DEGs were considered significant using a threshold of FDR<0.05. (C) shows example of Tier-3 classification in Endometrial cancer, which includes subgrouping based on an immune gene signature reflecting the tumour's immune landscape. The provided example stratifies patients using a immune risk score cut-off value as Low-immune risk score and High-immune risk score subgroups in Tier 1 Group 1 (Low AG-score) patients. Kaplan-Meier plot showing overall survival of Endometrial cancer patients. The immune risk score (AG-immune score) is derived using the following immune-associated genes from Table 5: CCL5, CD3D, CXCL9. CXCL10, GBP1, GZMB, and IDO1. The Tier 1 stratification was analysed according to the 5-genes (SRARP, IHH, TFF3, EYA4 and ANKRD33) risk score (AG-score). (D, E) shows identified biomarker(s) for the Tier-2 and Tier-4 subgroups. Tier-2 subgroups-specific tumour biology (pathway/biomarker analysis at protein/mRNA level) based on Reverse Phase Protein Array (RPPA) and log 2 mRNA expression data from TCGA Firehose legacy Uterine Corpus Endometrial cancer dataset (D) and TCGA Firehose legacy Prostate Adenocarcinoma dataset (E). Endometrial cancer Tier 2 Subgroups consist of the following genomic profiles: Subgroup 2 (PTEN-WT, TP53-WT), Subgroup 3 (PTEN-MUT, TP53-VVT), Subgroup 4 (PTEN-WT, TP53-MUT), Subgroup 5 (PTEN-MUT, TP53-MUT), Tier-4 classification step results in the identification of intermediate-risk cancer subgroups (those on the underside of each subgroup's median AG-score—Subgroups 2A, 3A, 4A and 5A) along with high-risk subgroups (those on upper-side of each subgroup's median AG-score—Subgroups 2B, 3B, 4B and 5B). Prostate cancer Tier 2 Subgroups consist of the following genomic profiles: Subgroup 2 (SPOP-VVT, TP53-WT), Subgroup 3 (SPOP-MUT, TP53-WT), Subgroup 4 (SPOP-WT, TP53-MUT), Subgroup 5 (SPOP-MUT, TP53-MUT),

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

It is noted that throughout the description cancer is provided as an example disease. This is done to help described the invention. However, a person skilled in the art would readily recognise that the methods described herein may be applied to any disease that includes genomic aberrations as described herein as part of the pathology of the disease. Therefore, the description should not be considered to be limited to cancer.

### Tier 1

There is provided herein one or more genomic aberrations associated with a disease, the method comprising:
a. identifying genes in a plurality of control subjects suffering from the disease that undergo a change of expression in response to a first genomic aberration and selecting the genes that undergo a change of expression in response to the first genomic aberration to provide a first set of differentially expressed genes (DEGs) associated with the first genomic aberration;
b. identifying DEGs of the first set of DEGs that overlap with DEGs of a control set of DEGs for a control genomic aberration and selecting the overlapping DEGs to form a first set of overlapping DEGs;
c. comparing the fold direction of change of expression of each DEG of the first set of overlapping DEGs to a fold direction change of expression of the corresponding DEG of the control set of DEGs;
d. classifying the first genomic aberration into a first or second group wherein:
   i. the first group comprises at least 51% overlapping DEGs that comprises a fold direction of change of expression that is the same as the fold direction of change of expression as the corresponding DEG of the control genomic aberration (Group A);
   ii. the second group comprises at least 51% overlapping DEGs that comprise a fold direction of change of expression that is inverse to the fold direction of change of expression of the corresponding DEG of the control genomic aberration (Group B).

In some example, the method may be a method stratifying a subject suffering from the disease or a disease that includes at least one genomic aberration associated with the disease. The classified genomic aberration is used to calculate a risk score for the subject and the subject is stratified based on the risk score; and
wherein the calculated risk score is indicative of prognosis of the subject.

Genomic aberrations as used herein may refers to any alteration to a subject's genetic information. For example, genomic aberrations may include gene mutations, such as point mutations, gene fusions, insertions, and/or deletions. In some examples, the mutations may be somatic mutations and/or germline mutations. In addition, genomic aberrations include somatic changes such as copy number alterations.

Association of a genomic aberration with a disease may be determined using any suitable method. For example, the genomic aberrations may associated with a disease by the occurrence of the genomic aberration in subjects suffering from the disease. For example, the genomic information of subjects suffering from the disease may be analysed and any genomic aberrations that are shown to be present in the subjects may be considered to be associated with disease. In some examples, the genomic aberration may occur in threshold number or fraction of subjects suffering from the disease. In some examples, the genomic aberration may not be or may be less prevalent in subjects not suffering from the disease. In some examples, the genomic aberrations may be the cause of the disease.

Detection of genomic aberrations in a subject or in each of the plurality of control subject may be by any method known in the field. For example, genomic aberrations may be detected by nucleic acid sequencing methods. In particular, DNA may be extracted from a sample, such as a tumour sample, from the subject to be utilized directly for identification of the individual's genomic aberrations. Particularly, examples of nucleic acid analysis methods are: direct sequencing or pyrosequencing, massively parallel sequencing, high-throughput sequencing (next generation sequencing), high performance liquid chromatography (HPLC) fragment analysis, capillarity electrophoresis and quantitative PCR (as, for example, detection by Taqman® probe, Scorpions™ ARMS Primer or SYBR Green). Several methods for detecting and analysing PCR amplification products are well known in the art. The general principles and conditions for amplification and detection of genomic aberrations, such as using PCR, are well known for the skilled person in the art.

Alternatively, other methods of nucleic acid analysis such as hybridization carried out using appropriately labelled probes, detection using microarrays e.g. chips containing many oligonucleotides for hybridization (as, for example, those produced by Affymetrix Corp.) or probe-less technologies and cleavage-based methods may be used. Amplification of DNA can be carried out using primers that are specific to the marker, and the amplified primer extension products can be detected with the use of nucleic acid probes. The DNA may be amplified by PCR prior to incubation with the probe and the amplified primer extension products can be detected using procedure and equipment for detection of the label.

In the case of genomic aberrations such as copy number variations, methods of detection may include paired-end mapping based detection (PE), split read based detection (SR), de novo assembly based detection (DA) and read depth based detection (RD). Other laboratory-based approaches may also be used for detecting CNVs, including multiplex ligation-dependent probe amplification (MLPA), microarray based comparative genomic hybridization (aCGH) and SNP microarrays, RNA sequencing, fluorescence in situ hybridization (FISH) and PCR based methods.

For example, for breast cancer the genomic aberrations may include any one or more of TP53 gene mutations, AHNAK2 gene mutations, AKAP9 gene mutations, BRCA1 gene mutations, DNAH11 gene mutations, FLG gene mutations, HERC2 gene mutations, copy number alterations, gene fusions, MUC16 gene mutations, PIK3R1 gene mutations, PTEN gene mutations, RB1 gene mutations, SYNE1 gene mutations, TTN gene mutations, USH2A gene mutations, PIK3CA gene mutations, AKT1 gene mutations, CBFB gene mutations, CDH1 gene mutations, FOXO3 gene mutations, GATA3 gene mutations, KMT2C gene mutations, MAP3K1 gene mutations, MUC12 gene mutations, MUC4 gene mutations, NCOR1 gene mutations, NF1 gene mutations, SF3B1 gene mutations, AHNAK gene mutations, DNAH2 gene mutations, KMT2D gene mutations, DNAH5 gene mutations, RYR2 gene mutations, PDE4DIP gene mutations, TG gene mutations, BIRC6 gene mutations, ERBB2 gene mutations, and/or BRCA2 gene mutations (see Table 1).

For example, for prostate cancer the genomic aberrations may include any one or more of TP53 gene mutations, TTN gene mutations, MUC16 gene mutations, NBPF1 gene mutations, SYNE1 gene mutations, copy number alterations, gene fusions, SPOP gene mutations, MUC17 gene mutations, and/or FOXA1 gene mutations (see Table 2).

For example, for endometrial cancer the genomic aberrations may include any one or more of TP53 gene mutations, CHD4 gene mutations, FBXW7 gene mutations, PTEN gene mutations, PIK3CA gene mutations, ARID1A gene mutations, KRAS gene mutations, MUC16 gene mutations, TTN gene mutations, PIK3R1 gene mutations, KMT2D gene mutations, CTNNB1 gene mutations, RYR2 gene mutations, CTCF gene mutations, SYNE1 gene mutations, copy number alterations, gene fusions, and/or ATM gene mutations (see Table 3).

Identification of genes that undergo a change in expression in response to a genomic aberration may be carried out by any suitable method known in the art. Differentially expressed” refers generally to a protein or nucleic acid (RNA, e.g. mRNA) that is overexpressed (upregulated) or underexpressed (downregulated) in one sample compared to at least one other sample. For example, in comparison to the expression level of one or more housekeeping genes in a control sample. For example, a control sample may be from a subject not suffering from the disease. Expression levels of genes may be normalized to housekeeping genes as is known in the field.

The terms “overexpress”, “overexpression”, “overexpressed” or “upregulated” interchangeably refer to a protein or nucleic acid (RNA) that is translated or transcribed at a detectably higher level, usually in a test sample, in comparison to a control or second test sample.

The term includes overexpression due to changes in transcription, post transcriptional processing, translation, post-translational processing, cellular localization (e.g., organelle, cytoplasm, nucleus, cell surface), RNA stability, protein stability, etc as compared to a control sample. Overexpression can be detected using conventional techniques for detecting RNA (i.e., RT-PCR, PCR, hybridization, RNA-Sequencing, NGS) or proteins (i.e., ELISA, immunohistochemical techniques). Overexpression can be an increase of 10%, 20%, 30%, 40%, 50%, 60%, 70%, 80%, 90% or more in comparison to the control. In certain instances, overexpression is an increase of 1-fold, 2-fold, 3-fold, 4-fold, or more in comparison to the control.

The terms “underexpress”, “underexpression”, “underexpressed” or “downregulated” interchangeably refer to a protein or nucleic acid (RNA) that is translated or transcribed at a detectably lower level, usually in a test sample, in comparison to a control or second test sample. The term includes underexpression due to changes in transcription, post transcriptional processing, translation, post-translational processing, cellular localization (e.g., organelle, cytoplasm, nucleus, cell surface), RNA stability and/or protein stability, as compared to a control sample. Underexpression can be detected using conventional techniques for detecting RNA (i.e., RT-PCR, PCR, hybridization, RNA-Sequencing, NGS) or proteins (i.e., ELISA, immunohistochemical techniques). Underexpression can be a decrease of 10%, 20%, 30%, 40%, 50%, 60%, 70%, 80%, 90% or more in comparison to the control. In certain instances, underexpression is a decrease of 1-fold, 2-fold, 3-fold, 4-fold, or more in comparison to the control.

Differential expression of genes may be determined by performing RNA expression analysis. RNA may be extracted from samples of a subject or plurality of subjects and the level of RNA may be quantified by hybridisation of probes to provide a gene expression value or count. The level of expression, gene count, of each gene may then be normalised based on the expression levels of a number of housekeeping genes by subtracting the average counts of the housekeeping genes from the counts of the gene of interest. The counts for the gene of interest may be expressed as log 10 or log 2 normalised gene counts to provide less skewed data. The level of expression, gene count of each gene may be normalised using a Z-score normalization method, which refers to the process of normalizing every count of the gene of interest in a dataset such that the mean of all of the counts is 0 and the standard deviation is 1 by using the following formula on every count of the gene of interest in a dataset: New value=(x−μ)/σ, where: x is Original count/expression value, μ is Mean count of gene of interest and a is Standard deviation of gene of interest expression values/count.

In some examples differential expression is evaluated by determining a magnitude of change in nucleic acid molecule or protein expression, to determine if gene or protein expression is up- or down-regulated. For example, a relative value of expression can be determined. In some examples, a decrease in the relative value of expression indicates that the gene or protein is downregulated, while an increase in the relative value of expression indicates that the gene or protein is upregulated.

Differential expression of genes and the expression levels of genes may be determined by any known methods and statistical analysis tools. For example, using RNAseq based methods such as DESeq, edgeR, NBPSeq, TSPM, baySeq, EBSeq, NOISeq, SAMseq and ShrinkSeq.

Differential expression of genes may be analysed using systems such as NanoString®, and Illumina HT 12 ®. Differential expression of genes may be analysed using the Limma R/Bioconductor software package, which calculates the p-value, adjusted p-value, and fold change for all the genes.

The plurality of control subjects may be a collection of any subjects who have been identified as suffering from the disease and have had a prognosis determined. Having a prognosis already determined allows for each group defined by the methods described herein to have a prognosis associated therewith.

For example, the plurality of control subjects and data as to genetic aberrations and/or DEGs associated therewith may be obtained from publicly or privately available disease specific datasets. For example, those available from the European Genome-Phenome Archive or The Cancer Genome Atlas (TCGA) program. For example, when the disease is breast cancer, the plurality of control subjects may include subjects and data associated therewith may include data from one or more of the Metabric Breast Cancer Datasets, TGCA Breast Cancer Datasets, The Metastatic Breast Cancer Project Dataset, CPTAC Proteogenomic landscape of Breast Cancer Dataset, SMC Breast Cancer Dataset, Breast Invasive Carcinoma Dataset from Broad Institute and Sanger Institute, and/or Breast GEO datasets, including GEO databases—GSE7390, GSE1456, GSE20685, and GSE9195. For example, when the disease is endometrial cancer, the plurality of control subjects may include subjects and data associated therewith from one or more of the TCGA Uterine Corpus Endometrial Carcinoma Datasets and/or CPTAC Endometrial Carcinoma Dataset. For example, when the disease is prostate cancer, the plurality of control subjects may include subjects and data associated therewith from one or more of the TCGA Prostate Adenocarcinoma Datasets, SU2C/PCF Dream Team Metastatic Prostate Adenocarcinoma Dataset, MSK/DFSI Prostate Adenocarcinoma Dataset and/or Fred Hutchinson CRC Prostate Adenocarcinoma Dataset.

The method described herein compares the DEGs associated with each genomic aberration to DEGs of a control genomic aberration. The control genomic aberration may be selected by analysis of genomic aberrations associated with the disease for the plurality of control subjects and determining the at least one, two, three, four, five, six, seven, eight, nine, ten, or more most commonly occurring genomic aberrations. In some examples, the control genomic aberration is selected based on the frequency of occurrence and/or the number of DEGs associated with the genomic aberration. For example, the control genomic aberration is selected from the most frequently occurring gene alterations or mutations for the disease. For example, the genomic aberration with the highest frequency of occurrence and/or highest number of statistically significant DEGs in a specific cancer. For example, the five, ten, fifteen or twenty or more most commonly occurring genomic aberrations associated with a disease may be determined and the control genomic aberration may be selected from one of these. In some examples, the control may be the most commonly occurring genomic aberration. In other examples, the control genomic aberration may be the second third, fourth fifth or more most commonly occurring genomic aberration and is selected based on the number of DEGs associated with the genomic aberration. In some examples, the control genomic aberration is selected based on the DEGs associated with the genomic aberration and the role these play in disease and/or subject. For example, the control genomic aberration may be selected based on the majority of significantly upregulated gene sets that are related to, for example, cell cycle, DNA replication, RNA transport, DNA repair, spliceosome and ribosome-biogenesis-associated molecular pathways. In the case of cancer the genes upregulated may be related to critical tumour growth-supporting pathways.

For example, when the disease is cancer the control genomic aberration may be mutations of TP53. For example, mutations of the TP53 encoding gene. The TP53 gene encodes a tumour suppressor protein (p53) containing transcriptional activation, DNA binding, and oligomerization domains. The encoded protein responds to diverse cellular stresses to regulate expression of target genes, thereby inducing cell cycle arrest, apoptosis, senescence, DNA repair, or changes in metabolism. TP53 mutations are universal across cancer types. TP53 is the most frequently mutated gene in human cancer. More than 50% of cancers involve a missing or damaged TP53 gene. The loss of a tumour suppressor is most often through large deleterious events, such as frameshift mutations, or premature stop codons. In TP53 however, many of the observed mutations in cancer are found to be single nucleotide missense variants. These variants are broadly distributed throughout the gene, but with the majority localizing in the DNA binding domain. There is no single hotspot in the DNA binding domain, but a majority of mutations occur in amino acid positions 175, 245, 248, 273, and 282 (NM_000546). To fulfil its proper biological function four TP53 polypeptides must form a tetramer which functions as a transcription factor, therefore even if one out of four polypeptides has inactivating mutation it may lead to dominant negative phenotype of variable degree.

Upon comparison of the DEGs of a genomic aberration to the DEGs control genomic aberration those DEGs that are shared between the genomic aberration and the control. These genes that are shared may be referred to as overlapping DEGs. The direction of change (i.e. upregulated or downregulated) for each of the overlapping genes is compared. For a genomic aberration having the majority of overlapping DEGs with the same direction of change (i.e. a DEG of the genomic aberration is upregulated and the same DEG of the control genomic aberration is also upregulated) the genomic aberration is classified as a Group A genomic aberration. If a genomic aberration has DEGs that have an opposite or inverse direction of change for the majority of DEGs when compared to the same DEGs for the control then the genetic aberration is classed as a Group B genomic aberration.

Once a first genomic aberration has been classified, further genomic aberrations associated with the disease can be classified. Once at least one Group A genomic aberration and at least one Group B genomic aberrations have been classified a representative Group A and Group B genomic aberration can be assigned. A representative genomic aberration is determined by analysing the frequency of occurrence of the genomic aberration and the number of DEGs associated with the genomic aberration. For example, in the case of cancer, TP53 gene mutations may be designated as the Group A representative. In the case of breast cancer, the representative Group B genomic aberration may be PIK3CA gene mutations. When the disease is prostate cancer the representative Group B genomic aberration may be SPOP gene mutations. When the disease is endometrial cancer the representative Group B genomic aberration may be PTEN gene mutations.

In some examples, further genomic aberrations may be designated or assigned to Group A or B by comparison to the representative Group A and B genomic aberration. For example, by comparing the number of DEGs associated with the further genomic aberration that have the same direction of change as the same DEGs of the Group A and B representative. In some examples, the further genomic aberration may have a similarity greater than 50% to both the representative genomic aberrations. In such examples, the further genomic aberration may be grouped based on the similarity. For example, if a further genomic aberration shares 7 DEGs with the DEGs of Group A and B genomic aberrations, and of these 4 have a direction of change the same as the Group A representative (and 3 that are inverse) giving a similarity of 57% and also shares 5 DEGs that have the same direction of change as the Group B representative giving a similarity of 71% the further genomic aberration may classified as Group B.

For example, when the disease is breast cancer, the Group A genomic aberrations may include one or more of TP53 gene mutations, AHNAK2 gene mutations, AKAP9 gene mutations, BRCA1 gene mutations, DNAH11 gene mutations, FLG gene mutations, HERC2 gene mutations, copy number alterations, gene fusions, MUC16 gene mutations, PIK3R1 gene mutations, PTEN gene mutations, RB1 gene mutations, SYNE1 gene mutations, TTN gene mutations, and/or USH2A gene mutations (see Table 1).

For example, when the disease is breast cancer, the Group B genomic aberrations may include one or more of PIK3CA gene mutations, AKT1 gene mutations, CBFB gene mutations, CDH1 gene mutations, FOXO3 gene mutations, GATA3 gene mutations, KMT2C gene mutations, MAP3K1 gene mutations, MUC12 gene mutations, MUC4 gene mutations, NCOR1 gene mutations, NF1 gene mutations, and/or SF3B1 gene mutations (see Table 1).

For example, when the disease is prostate cancer, the Group A genomic aberrations may include one or more of TP53 gene mutations, TTN gene mutations, MUC16 gene mutations, NBPF1 gene mutations, SYNE1 gene mutations, copy number alterations, and/or gene fusions (see Table 2).

For example, when the disease is prostate cancer, the Group B genomic aberrations may include one or more SPOP gene mutations, MUC17 gene mutations, and/or FOXA1 gene mutations (see Table 2).

For example, when the disease is endometrial cancer, the Group A genomic aberrations may include one or more of TP53 gene mutations, CHD4 gene mutations, FBXW7 gene mutations, copy number alterations, and/or gene fusions (see Table 3).

For example, when the disease is endometrial cancer, the Group B genomic aberrations may include one or more PTEN gene mutations, PIK3CA gene mutations, ARID1A gene mutations,

KRAS gene mutations, MUC16 gene mutations, TTN gene mutations, PIK3R1 gene mutations, KMT2D gene mutations, CTNNB1 gene mutations, RYR2 gene mutations, CTCF gene mutations, and/or SYNE1 gene mutations (see Table 3).

After assigning representative genomic aberrations, the DEGs that occur for both the representative Group A and Group B genomic aberrations the DEGs for each group can be compared and those DEGs that occur for both the Group A and Group B genomic aberrations are selected to provide a second set of overlapping DEGs (second set of shared DEGs).

In some examples, after assignment of the representative Group A and/or Group B genomic aberration, one or more further genomic aberrations designated as Group A or Group B may be compared as described to provide a second set of shared DEGs.

From the second set of shared DEGs a disease-specific gene signature is then selected. The DEGs selected for the disease-specific gene signature may be limited to these DEGs of the second shared to those DEGs that have a predefined statistical significance. For example, the disease-specific gene signature may only include DEGs that have a statistical analysis lower than a threshold value.

In some examples, further statistical approaches such as Lasso, univariate, and/or multivariate Cox regression analyses may be performed to identify the prognosis-related DEGs for a disease-specific gene signature.

In some examples, significantly upregulated genes or sets of genes associated with the control genomic aberration may be related to critical tumour growth-supporting pathways. For example, cell cycle, DNA replication, RNA transport, DNA repair, spliceosome and/or ribosome-biogenesis-associated molecular pathways.

In some examples, the threshold value may be a p-value calculated for the change in expression levels between a test sample (e.g. a tumour that includes the respective genomic aberration) and a control sample (e.g. a tumour sample that does not include the respective genomic aberration i.e. is wild-type in respect of the respective genomic aberration). This comparison may be carried out by any suitable method, for example, by using a two-sample t-test. For example, the threshold may be a FDR adjusted p-value (q-value). In some examples, the threshold may be an FDR adjusted p-value of at most 0.1. For example, the threshold may be an DR adjusted p-value of at most 0.1, 0.09, 0.08, 0.07, 0.06, 0.05, 0.04, 0.03, 0.02, or 0.01. In some examples, the DEGs used to form the disease specific gene signature may have an FDR adjusted p-value of about 0.01. In some examples, the DEGs used to form the disease specific gene signature may have an FDR adjusted p-value of less than 0.05.

The disease-specific gene signature includes at least two DEGs. In some examples, the disease-specific gene signature may include at least 2, 3, 4, 5, 6, 7, 8, 9, 10, 20, 30, 40, 50, 60, 70, 80, 90, 100, 200, 300, 400, 500 or more DEGs of the second shared set of DEGs. In some examples, the DEGs of the disease-specific gene signature include at least 2 DEGs that allow grouping (e.g. stratification) of subjects into at least two distinct groups. For example, the groups may be defined by clinical indications, such as overall survival, disease-free survival, distant metastasis-free survival (DMFS) and/or relapse-free survival. In some examples, the two groups may be considered a high-risk group (e.g. with lower probability of positive or non-improved clinical indications) and a low-risk group (e.g. with improved or comparatively higher probability of positive clinical indications).

In some examples, such as when the method is implemented by a computer or a database is formed as described herein, a disease specific gene signature is not defined until a later stage of the method and all the statistically significant DEGs of the second shared set are further analysed as described herein.

When a disease-specific gene signature is defined, at least one of DEGs selected has an inverse relationship of direction of change between Group A and Group B. That is to say that at least one DEG of the disease-specific gene signature has a fold direction of change of 1 for Group A and a fold direction of change of −1 for Group B or vice versa.

For example, the disease-specific gene signature may include at least 1, 2, 3, 4, 5, 6, 7, 8, 9, 10, 20, 30, 40, 50, 100, 200, 300, 400, 500, 1000 or more genes that have an inverse fold direction of change of expression between Group A and B.

In some examples, the disease-specific gene signature may include at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% genes that have an inverse fold direction of change of expression between Group A and B.

Once a disease-specific gene signature has been defined, the expression level of each of the DEGs (genes) included in the disease-specific gene signature is analysed. Expression level refers to the amount of RNA transcript and/or expression product produced for each gene of the disease-specific gene signature for each of the plurality of control subjects.

The amount RNA transcript and/or expression product may be a normalised amount that has been normalised as described above. For example, normalised to housekeeping genes or a Z-score normalisation is performed. In some examples, the expression level may include the amount of coding RNA transcript. In other examples, the expression level may include the amount of coding RNA and non-coding RNA produced from a gene. In some examples, the expression level may only include the amount of non-coding RNA.

The expression levels of genes may be determined using similar methods as described above for determining DEGs. In some examples, the expression level may be predetermined and part of the data provided for the plurality of control subjects from a previously analysed dataset.

The expression level of each DEG is then used to calculate an expression value or score for each gene based on the expression level. This may be done using a number of alternative methods.

For example, the expression values maybe normalised using known methods. For example, by taking the Log-2 transformed value of the expression level for each of the plurality of control subjects. Other methods of normalising the expression values may be used, such as taking the Log-10 transformed value or other Log-scaling methods, feature clipping, scaling to range, or using z-score.

In some examples, the normalised expression levels for each of the plurality of subjects for each gene of the disease-specific gene signature are ordered in ascending order (lowest to highest normalised expression level).

The ordered normalised expression levels are then divided into fractions based on the range of the expression values (for example, using data visualisation to visualise data through graphs and diagrams to see how expression values are distributed and whether or not it contains outliers) over all of the plurality of control subjects. For example, if the normalised expression levels have a range from 5 to 11 (for example, 5.2, 5.4, 5.5, 5.7, 6.2, 6.5, 6.9, 7.1, 7.3, 7.4, 7.9, and so on and up to 11—for example, 10.2, 10.3, 10.5, 10.8, 10.9), the expression values may be divided into six fractions (for example, the first fraction includes expression values 5.2, 5.4, 5.5, 5.7; the second fraction includes expression values 6.2, 6.5, 6.9; and so on and then last (sixth fraction) includes expression values, 10.2, 10.3, 10.5, 10.8, 10.9). If the normalised expression values range from 5 to 7, the normalised expression values may be divided into two fractions. The fractions are not required to be equal in size in order to maintain the variability of the data (for example, if the normalised expression levels have a range from 6 to 12 in a cohort of, 100 patients, and it has to be divided into five fractions then it is not required for the fractions to be equal in size—e.g. to have expression values from 20 patients in each fraction even when the expression value range in a fraction has a quite wide range of expression values, the expression values ranges from 9.2 to 11.9, for example, in the fifth fraction).

For all genes (of the disease-specific gene signature) that are upregulated for the representative Group A genomic aberration, the one or more further Group A genomic aberration and/or the control genomic aberration (for example, the DEG or gene has an increase in expression for the representative Group A genomic aberration, the one or more further Group A genomic aberration and/or the control genomic aberration and so is marked as an arbitrary value 1 in the overlapping set of genes and in the shared set of genes) a relative expression value (score) of 1 to n is assigned to each faction, where n is the fraction with the highest normalised expression level (e.g. assigned from lowest to highest). For example, if the normalised expression levels of a gene has been divided into 6 fractions, and the fold-direction of change is 1 (i.e. up-regulated in the representative Group A genomic aberration, the one or more further Group A genomic aberration and/or the control genomic aberration), then a relative expression value of 1 is assigned to the first fraction, a relative expression value of 2 is assigned to the second fraction, a relative expression value of 3 is assigned to the third fraction, a relative expression value of 4 is assigned to the fourth fraction, a relative expression value of 5 is assigned to the fifth fraction and a relative expression value of 6 is assigned to the sixth fraction.

For all genes (of disease-specific gene signature) that are downregulated for the representative Group A genomic aberration (and/or the control genomic aberration) (for example, the DEG or gene decrease in expression for the representative Group A genomic aberration the one or more further Group A genomic aberration and/or the control genomic aberration so is marked as an arbitrary value −1 in the overlapping set of genes and in the shared set of genes) a relative expression value (score) of 1 to n is assigned to each faction, where n is the fraction with the lowest normalised expression level (e.g. assigned from highest to lowest). For example, if the normalised expression levels of a gene has been divided into 6 fractions, and the fold-direction of change is −1 (i.e. down-regulated in the representative Group A genomic aberration, the one or more further Group A genomic aberration and/or the control genomic aberration), then a relative expression value of 6 is assigned to the first fraction, a relative expression value of 5 is assigned to the second fraction, a relative expression value of 4 is assigned to the third fraction, a relative expression value of 3 is assigned to the fourth fraction, a relative expression value of 2 is assigned to the fifth fraction and a relative expression value of 1 is assigned to the sixth fraction.

The scores of relative expression value assigned to each normalised expression level may then be used to assign a relative expression value to a subject. Therefore, the method may include providing the expression levels of a subject's genes of the disease-specific gene signature. As used herein, “provide”, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample). As used herein, the terms “biological sample”, “test sample”, “sample” and variations thereof refer to a sample obtained or derived from a subject.

For example, the expression levels of each gene of the disease-specific gene signature for the subject. This may be done using the expression level analysis methods provided above. For example, a sample may be taken from a subject and the expression levels determined from the sample.

The sample may be any suitable sample for assessing the disease. For example, if the disease is cancer, the sample may be taken from a tumour or cancerous cells. In the case of neurological diseases the sample may be taken from brain or nervous tissue. The sample may be a biological fluid sample, cell sample or a tissue sample.

In some examples, obtaining the sample is not part of the method as described herein. That is to say that the sample may have been taken previously and the expression levels for the genes being analysed may have already been calculated. Previously calculated expression levels can then be inputted into the method described herein.

The expression values of the subject's genes may be normalised using the same operation as described above for the expression levels of the plurality of control subjects. For example, the expression levels of the subject's genes are normalised by taking a Log-2 transformed value or performing a Z-score normalisation to provide a normalised expression level. The normalised expression level for the subject's gene is then compared to the ordered normalised expression value for the same gene of the plurality of control subjects. Based on what fraction the normalised expression value of the subject falls within, a score is assigned. For example, if the subject's normalised expression value is the same as a normalised expression value for one of the plurality of the control subjects in fraction 4, a score of 4 is assigned to the subject's gene.

This is then repeated for each gene of the disease-specific gene signature, and the score for each gene of the disease-specific gene signature is added together to provide a risk score for the subject.

As mentioned above, in some examples, all of the genes that make up the shared set of genes may analysed. In this case, the DEGs that make up the disease-specific gene signature are selected and the expression values for same DEGs as the disease-specific gene signature are retrieved from the database of scores for each gene already provided by the method above. The expression level of a subject's genes of disease-specific gene signature are provided and compared to the expression values for the plurality of control subjects in order to assign relative expression values to each of the genes of the disease-specific gene signature for the subject by selecting which fraction the subject's expression level for each gene of the disease-specific gene signature falls within.

In other examples, a score may be assigned to a gene by calculating a sum of the expression levels for each DEG of the disease-specific gene signature of the subject that are upregulated for corresponding DEG of the representative Group A genomic aberration, control genomic aberration and/or one of further group A aberrations and calculating the sum of the expression levels of each DEG of the disease-specific gene signature that are downregulated for the corresponding DEG of the representative Group A genomic aberration, about control genomic aberration and/or one of further group A aberrations and taking the difference between these two sums.

In some examples, the score may be calculated first by calculating weights from the effect of each gene included in the disease-specific gene signature on the clinical outcome (based on the coefficient value of each gene) through the following formula:

$$Score = {\sum }_{i = 1}^{n} Exp i * β i ,$$

where Expi is the expression value of each gene, and βi is the regression coefficient of the multivariate Cox analysis for each gene that makes up the disease-specific gene signature.

In another example, a score may be calculated by calculating the ratio of expression levels for the DEGs of the disease-specific gene signature of the subject that are upregulated for the corresponding DEG of representative Group A genomic aberration, control genomic aberration and/or one of further group A aberrations to the expression levels of each DEG of the disease-specific gene signature that are downregulated for the corresponding DEG of representative Group A genomic aberration, control genomic aberration and/or one of further group A aberrations.

In some examples, the efficiency of the prognostic disease-specific gene signature models developed using methods described herein may be assessed based on statistical parameters, for example, based on area under the curve (AUC) of receiver operating characteristic (ROC) curve, C-Index, Youden's index at 100% sensitivity (sensitivity+specificity−1), and/or reversed model size (1−nᵢ/n, where nᵢ is the number of genes in a defined disease-specific gene signature model, and n is the total number of prognostic disease-specific gene signature models). The model with the highest efficiency may be considered the best prognostic gene signature.

It is noted that Group B genomic aberrations are generally (though not always/not in all cancer types) associated with the downregulation of cancer-promoting pathways' signature genes, thus while calculating AG-score weightage is given to Group A genomic aberrations, i.e. with TP53 gene mutations. This implies that a higher expression of an RNA transcript that has a direction of association marked 1 (up-regulated in Group A genomic aberrations, i.e. with TP53 gene mutations) would correlate with a decreased likelihood of a good clinical outcome. Conversely, a higher expression of an RNA transcript with its direction of association marked −1 (down-regulated in Group A genomic aberrations, i.e. with TP53 gene mutations) would correlate with an increased likelihood of a good clinical outcome.

The risk score provides a first tier of stratification to the subject. The score can be used to stratify a subject into a high, low or intermediate risk group. For example, using statistical analysis of the risk score calculated for each of the plurality of control subjects for the same genes as the disease-specific gene signature, it is possible to determine a risk score that may be considered high or low for the disease-specific gene signature. For example, a high and low risk score for a disease-specific gene signature may be determined by the application of receiver-operating-characteristic (ROC) curve analysis to the scores calculated for the plurality of control subjects or, in some examples, based on the median risk score-based stratification method.

In some examples, Kaplan-Meier survival curves and log-rank test may be performed to evaluate the differences in the time to distant metastasis, disease-free survival and/or disease-specific overall survival of predicted good and poor prognosis groups.

### Tier 2

Once grouped into a high or low risk group the subject may be grouped into further subgroups or representative Group A and Group B genomic aberrations may be used to group into subgroups. For example, each of the low and high risk groups may be grouped into risk subgroups based on the genomic profile in relation to the representative Group A and Group B genomic aberrations of the subject. For example, the genomic profile may include the mutational status of the subject for the representative Group A and Group B genomic aberrations.

Mutational status refers to whether a subject suffers from a specified mutation or not. Mutations/aberrations may be detected using nucleic acid-based techniques as described above. In some examples, the high and low risk groups of the first tier may be sub-divided into multiple groups each. For example, the sub-groups may include:
a. representative Group A—mutant;
b. representative Group A—wild-type;
c. representative Group B—mutant;
d. representative Group B—wild-type;
e. representative Group A—mutant and representative Group B—mutant;
f. representative Group A—mutant and representative Group B—wild-type;
g. representative Group A—wild-type and representative Group B—mutant; and
h. representative Group A—wild-type and representative Group B—wild-type.

For example, when the disease is breast cancer, the risk subgroups for each of high and low risk groups of tier 1 may be:
a. TP53—mutant;
b. TP53—wild-type;
c. PIK3CA—mutant;
d. PIK3CA—wild-type;
e. TP53—mutant and PIK3CA—mutant;
f. TP53—mutant and PIK3CA—wild-type;
g. TP53—wild-type and PIK3CA—mutant; and
h. TP53—wild-type and PIK3CA—wild-type.

For example, when the disease is prostate cancer, the risk subgroups for each of high and low risk groups of tier 1 may be:
a. TP53—mutant;
b. TP53—wild-type;
c. SPOP—mutant;
d. SPOP—wild-type;
e. TP53—mutant and SPOP—mutant;
f. TP53—mutant and SPOP—wild-type;
g. TP53—wild-type and SPOP—mutant; and
h. TP53—wild-type and SPOP—wild-type.

For example, when the disease is endometrial cancer, the risk subgroups for each of high and low risk groups of tier 1 may be:
a. TP53—mutant;
b. TP53—wild-type;
c. PTEN—mutant;
d. PTEN—wild-type;
e. TP53—mutant and PTEN—mutant;
f. TP53—mutant and PTEN—wild-type;
g. TP53—wild-type and PTEN—mutant; and
h. TP53—wild-type and PTEN—wild-type.

Tier 2 groups may be further stratified by the mutational status of other Group A and/or Group B genomic aberrations. For example, genomic aberrations designated as Group A or B but that are not the representatives of each group. The other genomic aberrations used to further stratify a subject may be the second, third, fourth, fifth, sixth seventh, eighth, ninth, tenth or greater frequently occurring genomic aberration in group.

This further stratification of low- and high-risk groups results in subgroups with more specific biology and more accurate disease outcomes (prognosis). Tier 2 classification helps move away from the current ‘single-gene biomarker’ precision medicine strategy that focuses on single genetic alterations/mutations without understanding nuance, context and importance. The classification approach of the invention helps understanding of the nuance, context, significance and biology of the Group A and Group B frequent genomic aberrations in each disease type and identifies whether or not the genomic aberrations are in the “driver seat” when they are identified.

A high and low risk score for these specific subgroups may be determined using the methods described above in respect of the plurality of control subjects that fall within the same subgroups.

### Tier 3

An overwhelming amount of data from animal models and compelling data from human subjects indicate that a functional cancer immunosurveillance process can act as an extrinsic tumour suppressor. However, it has also become clear that the immune system can facilitate tumour progression, at least in part, by sculpting the immunogenic phenotype of tumours as they develop. The process by which immune cells modulate tumour progression, known as immunoediting, is a dynamic process that creates a selective pressure that finally leads to the generation of immune-resistant cells and the inability of the immune system to eradicate a tumour (tumour immunosuppression).

Tumour immunosuppression describes the suppressed host immune responses to tumour antigens, resulting in the reduction or loss of antigens on tumour cells, inhibiting the activation of immune effector cells and decreased cell viability of cytotoxic T lymphocytes (CTLs) or natural killer cells. Sometimes, tumours develop various tactics to suppress antitumor immunity, leading to the failure of immune regulation of tumour growth. These tactics include expressing a series of receptors on the tumour cell surface, called immune checkpoints or immunosuppressive ligands; e.g., PD-L1, a transmembrane surface antigen with an immunoglobulin-like structure, is distributed in many tissues and interactions with PD-L1 lead to inhibition of T-cell receptor-mediated T-cell activation. In addition, other immune checkpoints (such as FAS-L and IDO) have also been reported to inhibit T-cell responses by depleting tryptophan and producing kynurenine (toxic to lymphocytes) or mediating activation-induced cell death. Nevertheless, different types of cancer express diverse immune checkpoints and even in the same type of tumour, the expression of immune checkpoints is different across patients.

In this context, the cellular and functional characterisation of the immune compartment within a tumour microenvironment can help to understand tumour progression and, ultimately, create novel predictive and prognostic tools and improve subject stratification for cancer treatment as well as for other diseases involving the immune system.

Following the tier 2 classification (that involves subgrouping the high and low-risk groups based on the subject's genomic profiles), the invention applies a third tier of stratification that includes subgrouping based on an immune gene signature reflecting the immune landscape of a subject by determining an immune score.

In some examples, an immune risk score (immune score) maybe used alone to stratify subjects. As mentioned above, in other examples immune risk score may be used to stratify subjects already grouped into risk groups and/or sub-risk groups.

Therefore, there is also provided herein a method of calculating an immune risk score for a subject suffering from a disease, the disease comprising one or more genomic aberrations, the method comprising:
a. selecting two or more or three or more immune associated genes associated with disease to form at least one immune signature;
b. assigning a direction of association to each gene of the immune signature based on a change of expression for a plurality of control subjects;
c. wherein each gene with an increased expression level is designated with a direction of association and each gene with a decreased expression is designated with a second direction of association inverse to the first direction of association;
d. determining an expression level for each gene of the immune signature based on a level of RNA transcript for each gene for the plurality of control subjects;
e. providing an expression level for each gene of the immune signature based on a level of RNA transcript for each gene for the subject;
f. calculating an immune risk score based on the direction of association and the expression level of the each gene of the immune signature of the subject;
g. wherein the subject is stratified as a high immune risk, a low immune risk or an intermediate immune risk based on the calculated immune score; and
h. wherein the immune risk score is indicative of a prognosis of the subject

Calculating an immune risk score may be done in a similar manner as described above for the risk score. For example, the expression level of each of the genes included in the immune signature is analysed. Expression level refers to the amount of RNA transcript and/or expression product produced for each gene of the immune signature for each of the plurality of control subjects.

The amount RNA transcript and/or expression product may be a normalised amount that has been normalised as described above. For example, normalised to housekeeping genes or a Z-score normalisation is performed. In some examples, the expression level may include the amount of coding RNA transcript. In other examples, the expression level may include the amount of coding RNA and non-coding RNA produced from a gene. In some examples, the expression level may only include the amount of non-coding RNA.

The expression levels of genes may be determined using similar methods as described above. In some examples, the expression level may be predetermined and part of the data provided for the plurality of control subjects from a previously analysed dataset.

The expression level of each gene is then used to calculate an expression value or score for each gene based on the expression level. This may done using a number of alternative methods.

For example, the expression values maybe normalised using known methods such as those described above.

In some examples, the normalised expression levels for each of the plurality of subjects for each gene of the immune signature are ordered in ascending order (lowest to highest normalised expression level).

The ordered normalised expression levels are then divided into fractions based on the range of the expression values over all of the plurality of control subjects. For example, if the normalised expression levels have a range from 5 to 11, the expression values may be divided into 6 fractions. If the range of the normalised expression values is from 5 to 7, the normalised expression values may be divided into 2 fractions. The fractions are not required to be equal in size in order to maintain the variability of the data.

For all genes that have a first direction of association (for example up-regulated genes) a relative expression value (score) of 1 to n is assigned to each faction, where n is the fraction with the highest normalised expression level (e.g. assigned from lowest to highest). For example, if the normalised expression levels of a gene has been divided into 6 fractions, and the first direction of association is up-regulation, then a relative expression value of 1 is assigned to the first fraction, a relative expression value of 2 is assigned to the second fraction, a relative expression value of 3 is assigned to the third fraction, a relative expression value of 4 is assigned to the fourth fraction, a relative expression value of 5 is assigned to the fifth fraction and a relative expression value of 6 is assigned to the sixth fraction.

For all genes that have a direction of association that is inverse to the first direction of association (i.e. down-regulated) a relative expression value (score) of 1 to n is assigned to each faction, where n is the fraction with the lowest normalised expression level (e.g. assigned from highest to lowest). For example, if the normalised expression levels of a gene has been divided into 6 fractions, and the fold-direction of change is the inverse in comprising to the control genomic aberration, then a relative expression value of 6 is assigned to the first fraction, a relative expression value of 5 is assigned to the second fraction, a relative expression value of 4 is assigned to the third fraction, a relative expression value of 3 is assigned to the fourth fraction, a relative expression value of 2 is assigned to the fifth fraction and a relative expression value of 1 is assigned to the sixth fraction.

The scores of relative expression value assigned to each normalised expression level may then be used to assign a relative expression value to a subject. Therefore, the method may include providing the expression levels of a subject's genes of the immune signature from a subject sample as described above.

In some examples, obtaining the sample is not part of the method as described herein. That is to say that the sample may have been taken previously and the expression levels for the genes being analysed may have already been calculated. Previously calculated expression levels can then be inputted into the method described herein.

The expression values of the subject's genes may be normalised using the same operation as described above for the expression levels of the plurality of control subjects. For example, the expression levels of the subject's genes are normalised by taking a Log-2 transformed value or performing a Z-score normalisation to provide a normalised expression level. The normalised expression level for the subject's gene is then compared to the ordered normalised expression value for the same gene of the plurality of control subjects. Based on what fraction the normalised expression value of the subject falls within, a score is assigned. For example, if the subject's normalised expression value is the same as a normalised expression value for one of the plurality of the control subjects in fraction 4, a score of 4 is assigned to the subject's gene.

This is then repeated for each gene of the immune signature and the score for each gene of the immune signature is added together to provide an immune risk score for the subject.

In some examples, the genes that make up the immune signature are selected and the expression values for same genes as the immune signature are retrieved from a database of scores for each gene already provided by the method above. The expression level of a subject's genes of the immune signature are provided and compared to the expression values for the plurality of control subjects in order to assign relative expression values to each of the genes of the immune signature for the subject by selecting which fraction the subject's expression level for each gene of the immune signature falls within.

In other examples, a score may be assigned to a gene by calculating a sum of the expression levels for each gene of the immune signature of the subject having a first direction of association of expression and calculating the sum of the expression levels of each gene of the immune signature having a second direction of association of expression that is inverse to the first direction of association and taking the difference between these two sums.

In some examples, the immune risk score may be calculated first by calculating the weights from the effect of each gene included in the immune signature on the clinical outcome (based on the coefficient value of each gene) through the following formula: Immune score=ΣExpi*βi, where Expi is the expression value of each gene, and βi is the regression coefficient of the multivariate Cox analysis for each gene that makes up the immune signature.

In another example, a score may be calculated by calculating the ratio of expression levels for the each gene of the immune signature of the subject having a first direction of association of expression to the expression levels of each gene of the immune signature having a direction of association of expression that is inverse to first direction of association.

In some examples, the efficiency of the immune signature models developed using methods described above may be assessed based on statistical parameters, for example, based on area under the curve (AUC) of receiver operating characteristic (ROC) curve, C-Index, Youden's index at 100% sensitivity (sensitivity+specificity−1), and/or reversed model size (1−nᵢ/n, where nᵢ is the number of genes in a defined disease-specific immune signature model, and n is the total number of immune signature models). The model with the highest efficiency may be considered the best prognostic immune gene signature.

The immune risk score provides a standalone method of stratification of the subject or may be used to further stratify any of the tier 1 or tier 2 groups described above. The immune risk score can be used to stratify a subject into a high, a low or an intermediate risk group. For example, using statistical analysis of the immune risk score calculated for each of the plurality of control subjects for the same genes as the immune signature it is possible to determine an immune risk score that may be considered high or low for the immune signature. For example, a high and low immune risk score for an immune signature may be determined by application of receiver-operating-characteristic (ROC) curve analysis to the scores calculated for the plurality of control subjects or, in some examples, based on the median immune score-based stratification method.

Immune associated genes and an immune gene signature may be determined by analysis for immune genes that have a change of expression in the plurality of control subjects. In some examples, an immune gene signature may be determined by review of literature relating to a specific disease that identified specific immune genes that have altered expression in subjects suffering from the disease. In some examples, statistical approaches such as Lasso, univariate, and/or multivariate Cox regression analyses may be performed to identify prognosis-related immune genes for a disease-specific immune signature.

In some examples, such as for cancer, the immune associated genes may comprise one or more of the genes listed in Table 5. In some examples, immune associated genes may comprise a plurality of the genes listed in Table. 5

For example, for breast cancer the immune associated genes may include one or more of CCL5, CD3D, CXCL9, CXCL10, GBP1, GBP4, GBP5, GZMB, IDO1, NFS1, NKG7, CD247, CD7, CTLA4, CD2, CD38, ICOS, GZMA, GNLY, IL18BP, CD8A, TCRVB, PTPRCAP, CXCR6, SH2D1A, CXCR3, PRF1, PVRIG, ITK, HCST, LTA, PYHIN1, IRF1, MAP4K1, CD3G, PRKCB, CD48, IL21R, TAP1, CD6.

For example, for endometrial cancer the immune associated genes may include one or more of CCL5, CD3D, CXCL9, CXCL10, GBP1, GBP4, GBP5, GZMB, IDO1, NFS1, NKG7, CD247, CD7, CTLA4, CD2, CD38, ICOS, GZMA, GNLY, IL18BP, CD8A, TCRVB, PTPRCAP, CXCR6, SH2D1A, CXCR3, PRF1, PVRIG, ITK, HCST, LTA, PYHIN1, IRF1, MAP4K1, CD3G, PRKCB, CD48, IL21R, TAP1, CD6.

Cut-off values for classifying subjects, groups and/or subgroups as low or high-immune risk score subgroups can be determined using methods known in the art, such as ROC analysis as described above or, in some examples, based on the median immune score-based stratification method.

In some examples, a high immune risk score may be an indication of activation of adaptive and/or innate immunity in a subject.

The sub-classification of tier 2-subgroups using an additional third tier of stratification (i.e. immune score) further refines the biology and prognosis of tier 2 subgroups. In addition, tier 3 stratification may further identify low-risk subject subgroups (for example with >90% 20-year overall survival probability) from high-risk subjects group identified from the tier 2 stratification. For example, in an untreated early ER+ breast cancer cohort (that received no systemic adjuvant therapy), the additional third tier of stratification of high-risk tier 2-subgroups may identify low-risk subject subgroups with high immune-score and >90% 20-year overall survival probability. These newly recognised low-risk subject subsets identified from the high-risk tier 2 subgroups could thus avoid overdiagnosis and overtreatments, given the >90% 20-year overall survival probability even without any systemic therapies.

Also, the further sub-stratification of tier 2-subgroups using the described additional third tier of stratification (i.e. immune score) may help identify high-risk subject subgroups (with <90% 10-year overall survival probability) from tier 2 low-risk subject subgroups. For example, in an endometrial cancer cohort, the additional third tier of stratification of tier 2 low-risk subgroup having the mutational status of PTEN-mutant (PTEN-MUT) can identify a high-risk subject subgroup with low immune risk score and <90% 10-year overall survival probability.

In some examples, immune score-based stratification can also be used to select subjects who would or would not benefit from certain therapies, such as adjuvant therapies or immune checkpoint inhibitors.

In some examples, immune risk score may also have usefulness in tailoring immunotherapies and designing rational combination therapy strategies for improved responses. For example, in endometrial cancer, the invention may help identify subgroups wherein, despite having an active immune system (having a high immune score), the immune response in the subgroup is not optimal (or functional) given there is no significant prognosis benefit in the high immune score-subgroup compared to the low immune score-counterpart. In general, response to checkpoint inhibitors is better if tumours show high-level microsatellite instability (MSI-high) caused by mismatch repair deficiency. Given subgroup 4 (with PTEN-WT, TP53-MUT genomic profile) in endometrial cancers expresses a high level of proteins involved in DNA mismatch repair (MSH2 and MSH6), a rational combination therapy strategy for improved response could be providing immune checkpoint inhibitors and the inhibitors of the DNA mismatch repair enzymes.

In some examples, immune risk score may identify subjects with the active immune system from specific subgroups with elevated immunogenicity that will not require any systemic adjuvant therapies given a >90% 20-year overall survival prediction and thus may help prevent or avoid overdiagnosis and overtreatment.

### Tier 4

The invention may also include further stratification of subjects based on the median-score. For example, a tier group or subgroup may be further stratified based on the median score for each or multiple groups or subgroups. For example, the median score for the plurality of control subjects in a high-risk group (Tier 1), high risk subgroup (tier 2) and having a high immune risk score may be calculated and compared with a risk score of a subject stratified into the same groups. This further classification step (Tier-4) results in the identification of intermediate-risk cancer subgroups (those on the underside of each subgroup's median AG-score) along with extremely high-risk subgroups (those on the upper side of each subgroup's median AG-score) that are characterised by relatively better prognosis and extremely poor prognosis, respectively. This further classification step (Tier-4) in a low-risk group (Tier 1) results in the identification of intermediate-risk cancer subgroups (those on the upper side of each subgroup's median AG-score) along with extremely low-risk subgroups (those on the underside of each subgroup's median AG-score) that are characterised by relatively poor prognosis and extremely good prognosis, respectively.

In some examples, subjects having a risk score higher than the median-risk score may be further stratified into a high risk subset (extremely high-risk subgroups—Tier 4). In some examples, subjects having a risk score lower than the median-risk score may be further stratified into a low risk subset (intermediate-risk subgroups—Tier 4).

The median-risk score of each group and/or subgroup may be based on each group or subgroup's median risk score plus or minus 1, 2, 3, 4, or 5 median risk score. For example, if the median score in a subgroup is 16, then the cut-off values for subdivision into Tier 4 subgroups e.g. high risk subset or extremely high-risk subgroups and a low risk subset or intermediate-risk subgroups could be 16, 16+1 (i.e. 17), 16+2 (i.e. 18) or 16-1 or 16-2 and so on.

### Tier 5

In some examples, the method encompasses an additional fifth-tier of classification that involves classifying the above-identified groups, subgroups and/or subsets from tier 1, tier 2, tier 3 and/or tier 4 (respectively) stratification steps into further subgroups (sub-risk groups) based on the mutational status of genes (from Group A and Group B genomic aberrations list) not already directly included in the tier 2 stratification. For example, for breast cancer, this would involve sub classifying groups, subgroups and/or subsets into further subgroups based on, for instance, the tumour's CDH1 or MAP3K1 or GATA3 gene mutation statuses. For example, for prostate cancer, this would involve subclassifying groups, subgroups and/or subsets into further subgroups based on, for instance, the tumour's MUC17 or FOXA1 or TTN or MUC16 gene mutation statuses.

Incorporating the tier 5 classification step makes the present classification method an open-ended approach whereby any additional frequent genomic aberrations in cancer could be accommodated in the classification method, and their precise role in the cancer type could be determined. This reflects the utility of the present multi-tier classification method for precision medicine purposes in its truest sense.

### Tier 6

Metastatic tumour progression is a multistep process that begins with the local invasion of the primary tumour into the surrounding tissue, accompanied by the spreading of cancer cells through lymphatics and blood vessels, producing metastases at distant locations.

First and second-generation prognostic signatures are dominated by proliferation pathways. As mentioned above, shared sets of genes associated with Tier 1 disease-specific gene signatures may also be related to critical tumour growth-supporting pathways, for example, cell cycle and proliferation. Tumour dissemination and metastasis are associated with cancer-progression-specific pathways, including invasion, Epithelial-Mesenchymal Transition (EMT), metastasis, intravasation and dissemination. Proliferation is a rate-limiting step of distant colonisation and is thus particularly important for assessing cancer prognosis. A prognostic score derived from a signature of dissemination (or metastasis) could provide complementary and more personalised prognostic and predictive information for patients with a disease such as cancer.

In some examples, the methods described herein encompass or comprise an additional sixth-tier of classification that involves classifying the above-identified groups, subgroups and/or subsets from tier 1, tier 2, tier 3, tier 4 and/or tier 5 (respectively) stratification steps into further subgroups (sub-risk groups) based on a metastatic risk score (metastatic score) derived from a signature of dissemination. For example, for cancer, this would involve subclassifying groups, subgroups and/or subsets into further subgroups based on, for instance, the metastatic risk score derived using the signature of dissemination.

In some examples, the genes and/or proteins associated with metastasis and dissemination are identified in the abundance of the genes/proteins belonging to the dominant proliferation pathways through the analysis of comparing primary tumours from non-metastatic tumours with the primary tumours from patients that develop metastatic tumours separately for each of the low and high risk groups identified at Tier 1 level and identifying DEGs and/or differentially expressed proteins within each of these two groups that would together constitute the signature of dissemination. In other examples, a similar analysis between the primary tumours from non-metastatic and patients that develop metastatic tumours may be performed between any of the tier 1, tier 2, tier 3, tier 4 and/or tier 5 groups described above.

In some examples, the genes and/or proteins associated with metastasis and dissemination are identified in the abundance of the genes/proteins belonging to the dominant proliferation pathways by comparing primary tumours from lymph node-negative patients with the primary tumours from patients with lymph node metastasis separately for each of the low and high risk groups identified at Tier 1 level and identifying DEGs and/or differentially expressed proteins within each of these two groups that would together constitute the signature of dissemination. In some examples, the analysis is performed between the primary tumours from lymph node-negative patients who had no event before 10 years after diagnosis with primary tumours from patients with lymph node metastasis who had an event before 10 years after diagnosis for any of the tier 1, tier 2, tier 3, tier 4 and/or tier 5 groups described above.

From the identified DEGs/proteins, a disease-specific gene metastatic signature is then selected. The DEGs selected for the disease-specific metastatic gene signature may be limited to those with predefined statistical significance. For example, the disease-specific metastatic gene signature may only include DEGs with a statistical analysis lower than a threshold value.

In some examples, statistical approaches such as Lasso, univariate, and/or multivariate Cox regression analyses may be performed to identify prognosis/survival-related DEGs for a disease-specific metastatic gene signature.

In some examples, a metastatic risk score maybe used alone to stratify subjects. As mentioned above, in other examples metastatic risk score may be used to stratify subjects already grouped into risk groups and/or sub-risk groups.

Therefore, there is also provided herein a method of calculating a metastatic risk score for a subject suffering from a disease, the disease comprising one or more genomic aberrations, the method comprising:
a. selecting two or more metastasis/dissemination-associated genes associated with the disease to form at least one disease-specific metastatic signature (metastatic signature);
b. assigning a direction of association to each gene of the disease-specific metastatic signature based on a change of expression for a plurality of control subjects;
c. wherein each gene with an increased expression level is designated with a direction of association and each gene with a decreased expression is designated with a second direction of association inverse to the first direction of association;
d. determining an expression level for each gene of the disease-specific metastatic signature based on a level of RNA transcript for each gene for the plurality of control subjects;
e. providing an expression level for each gene of the disease-specific metastatic signature based on a level of RNA transcript for each gene for the subject;
f. calculating a metastatic risk score based on the direction of association and the expression level of each gene of the disease-specific metastatic signature of the subject;
g. wherein the subject is stratified as a high metastatic risk, a low metastatic risk or an intermediate metastatic risk based on the calculated metastatic score; and
h. wherein the metastatic risk score is indicative of a prognosis of the subject.

The method may also include performing statistical analysis such as Lasso, univariate, and/or multivariate Cox regression analyses to identify prognosis-related metastatic/dissemination associated genes for a disease-specific metastatic signature. For example, this may be done after step (a) and before step (b) above.

Calculating a metastatic risk score may be done in a similar manner as described above for the risk score and immune score. For example, the expression level of each of the genes included in the metastatic signature is analysed. Expression level refers to the amount of RNA transcript and/or expression product produced for each gene of the metastatic signature for each of the plurality of control subjects.

The amount RNA transcript and/or expression product may be a normalised amount that has been normalised as described above. For example, normalised to housekeeping genes or a Z-score normalisation is performed. In some examples, the expression level may include the amount of coding RNA transcript. In other examples, the expression level may include the amount of coding RNA and non-coding RNA produced from a gene. In some examples, the expression level may only include the amount of non-coding RNA.

The expression levels of genes may be determined using similar methods as described above. In some examples, the expression level may be predetermined and part of the data provided for the plurality of control subjects from a previously analysed dataset.

The expression level of each gene is then used to calculate an expression value or score for each gene based on the expression level. This may done using a number of alternative methods as described above for the risk score and immune score.

For example, the expression values maybe normalised using known methods such as those described above.

In some examples, a score may be assigned to a gene by calculating a sum of the expression levels for each gene of the metastatic signature of the subject having a first direction of association of expression and calculating the sum of the expression levels of each gene of the metastatic signature having a second direction of association of expression that is inverse to the first direction of association and taking the difference between these two sums.

In some examples, the metastatic risk score may be calculated first by calculating weights from the effect of each gene included in the metastatic signature on the clinical outcome (based on the coefficient value of each gene) through the following formula: Metastatic score=Σᵢ₌₁ Expi*βi, where Expi is the expression value of each gene, and βi is the regression coefficient of the multivariate Cox analysis for each gene that makes up the metastatic signature.

In another example, a score may be calculated by calculating the ratio of expression levels for the each gene of the metastatic signature of the subject having a first direction of association of expression to the expression levels of each gene of the metastatic signature having a direction of association of expression that is inverse to first direction of association.

In some examples, the efficiency of the disease-specific metastatic signature models developed using methods described above may be assessed based on various statistical parameters, for example, based on area under the curve (AUC) of receiver operating characteristic (ROC) curve, C-Index, Youden's index at 100% sensitivity (sensitivity+specificity−1), and/or reversed model size (1−nᵢ/n, where nᵢ is the number of genes in a defined metastatic signature model, and n is the total number of metastatic signature models). The model with the highest efficiency may be considered the best prognostic metastatic signature.

The metastatic risk score provides a standalone method of stratification of the subject or may be used to stratify further any of the tier 1, tier 2, tier 3, tier 4 and/or tier 5 groups described above. The metastatic risk score can be used to stratify a subject into a high, a low or an intermediate risk group. For example, using statistical analysis of the metastatic risk score calculated for each of the plurality of control subjects for the same genes as the metastatic signature it is possible to determine a metastatic risk score that may be considered high or low for the metastatic signature. For example, a high and low metastatic risk score for an metastatic signature may be determined by application of receiver-operating-characteristic (ROC) curve analysis to the scores calculated for the plurality of control subjects or, in some examples, based on the median metastatic score-based stratification method.

Metastasis-associated genes and metastatic gene signatures may be determined by analysis for metastatic genes that have a change of expression in the plurality of control subjects. In some examples, a metastatic gene signature may be determined by a review of literature relating to a specific disease that has identified specific metastatic genes that have altered expression in subjects suffering from the disease. In some examples, such as for breast cancer, the metastasis/dissemination associated genes may comprise one or more of the genes listed in Table 8. In some examples, metastasis/dissemination associated genes may comprise a plurality of the genes listed in Table 8.

### Combinations

The methods described herein (such as the methods described above, methods of determining or predicting prognosis and/or computer implemented methods) may comprise carrying out analysis according to at least one of Tier 1, Tier 2, Tier 3, Tier 4, Tier 5, and/or Tier 6 as described herein. In one example, the methods described herein include carrying out analysis according to Tier 1. In one example, the methods described herein include carrying out analysis according to Tier 2. In one example, the methods described herein include carrying out analysis according to Tier 3. In one example, the methods described herein include carrying out analysis according to Tier 5. In one example, the methods described herein include carrying out analysis according to Tier 6.

In one example, the methods described herein include carrying out analysis according to Tier 1, and Tier 2. For example, the methods described herein may include calculating a risk score of a subject as described herein stratifying the subject into one or more risk groups and stratifying the subject into one or more risk subgroups (Tier 2 groups) based on the mutational status of the representative Group A and/or Group B genomic aberrations.

In one example, the methods described herein include carrying out analysis according to Tier 1, and Tier 3. For example, the methods described herein may include calculating a risk score of a subject as described herein stratifying the subject into one or more risk groups and stratifying the subject based on the risk score and calculating an immune risk score and further stratifying the subject base on the immune risk score. In some examples, the method may include carrying out tier 1 analysis first and then subsequently carrying out tier 3 analysis. In some examples, the method may include carrying out tier 3 analysis first and then subsequently carrying out tier 1 analysis.

In one example, the methods described herein include carrying out analysis according to Tier 1, and Tier 4. For example, the methods described herein may include calculating a risk score of a subject as described herein, stratifying the subject into one or more risk groups and further stratifying the subject into an intermediate or high risk or low risk subset (Tier 4) based on a median risk score.

In one example, the methods described herein include carrying out analysis according to Tier 1, and Tier 5. For example, the methods described herein may include calculating a risk score of a subject as described herein stratifying the subject into one or more risk groups and stratifying the subject based on the mutational status of one or more additional Group A and/or Group B genomic aberrations to provide sub-risk groups (Tier 5).

In one example, the methods described herein include carrying out analysis according to Tier 1, and Tier 6. For example, the methods described herein may include calculating a risk score of a subject as described herein stratifying the subject into one or more risk groups stratifying the subject base on the risk score and calculating a metastatic risk score and stratifying the subject based on the metastatic risk score. In some examples, the method may include carrying out tier 1 analysis first and then subsequently carrying out tier 6 analysis. In some examples, the method may include carrying out tier 6 analysis first and then subsequently carrying out tier 1 analysis.

In one example, the methods described herein include carrying out analysis according to Tier 2, and Tier 3. For example, the methods described herein may include stratifying the subject into one or more risk subgroups (Tier 2 groups) based on the mutational status of the representative Group A and/or Group B genomic aberrations and calculating an immune risk score and further stratifying the subject base on the immune risk score. In some examples, the method may include carrying out tier 2 analysis first and then subsequently carrying out tier 3 analysis. In some examples, the method may include carrying out tier 3 analysis first and then subsequently carrying out tier 2 analysis.

In one example, the methods described herein include carrying out analysis according to Tier 2, and Tier 4. For example, the methods described herein may include stratifying the subject into one or more risk subgroups (Tier 2 groups) based on the mutational status of the representative Group A and/or Group B genomic aberrations and further stratifying the subject into an intermediate or high risk subset (Tier 4) based on a median risk score. In some examples, the method may include carrying out tier 2 analysis first and then subsequently carrying out tier 4 analysis.

In one example, the methods described herein include carrying out analysis according to Tier 2, and Tier 5. For example, the methods described herein may include stratifying the subject into one or more risk subgroups (Tier 2 groups) based on the mutational status of the representative Group A and/or Group B genomic aberrations and stratifying the subject based on the mutational status of one or more additional Group A and/or Group B genomic aberrations to provide sub-risk groups (Tier 5). In some examples, the method may include carrying out tier 2 analysis first and then subsequently carrying out tier 5 analysis. In some examples, the method may include carrying out tier 5 analysis first and then subsequently carrying out tier 2 analysis.

In one example, the methods described herein include carrying out analysis according to Tier 2, and Tier 6. For example, the methods described herein may include stratifying the subject into one or more risk subgroups (Tier 2 groups) based on the mutational status of the representative Group A and/or Group B genomic aberrations and calculating a metastatic risk score and stratifying the subject base on the metastatic risk score. In some examples, the method may include carrying out tier 2 analysis first and then subsequently carrying out tier 6 analysis. In some examples, the method may include carrying out tier 6 analysis first and then subsequently carrying out tier 2 analysis.

In one example, the methods described herein include carrying out analysis according to Tier 3, and Tier 4. For example, the methods described herein may include calculating an immune risk score and further stratifying the subject base on the immune risk score and further stratifying the subject into an intermediate or high risk subset (Tier 4) based on a median immune risk score. In some examples, the method may include carrying out tier 3 analysis first and then subsequently carrying out tier 4 analysis. In some examples, the method may include carrying out tier 4 analysis first and then subsequently carrying out tier 3 analysis.

In one example, the methods described herein include carrying out analysis according to Tier 3, and Tier 5. For example, the methods described herein may include calculating an immune risk score and further stratifying the subject based on the immune risk score and further stratifying the subject based on the mutational status of one or more additional Group A and/or Group B genomic aberrations to provide sub-risk groups (Tier 5). In some examples, the method may include carrying out tier 3 analysis first and then subsequently carrying out tier 5 analysis. In some examples, the method may include carrying out tier 5 analysis first and then subsequently carrying out tier 3 analysis.

In one example, the methods described herein include carrying out analysis according to Tier 3, and Tier 6. For example, the methods described herein may include calculating an immune risk score and further stratifying the subject based on the immune risk score and calculating a metastatic risk score and stratifying the subject based on the metastatic risk score. In some examples, the method may include carrying out tier 3 analysis first and then subsequently carrying out tier 6 analysis. In some examples, the method may include carrying out tier 6 analysis first and then subsequently carrying out tier 3 analysis.

In one example, the methods described herein include carrying out analysis according to Tier 4, and Tier 5. For example, the methods described herein may include stratifying the subject into a low risk or high risk subset (Tier 4) based on a median risk score and further stratifying the subject based on the mutational status of one or more additional Group A and/or Group B genomic aberrations to provide sub-risk groups (Tier 5). In some examples, the method may include carrying out tier 4 analysis first and then subsequently carrying out tier 5 analysis. In some examples, the method may include carrying out tier 5 analysis first and then subsequently carrying out tier 4 analysis.

In one example, the methods described herein include carrying out analysis according to Tier 4, and Tier 6. For example, the methods described herein may include stratifying the subject into a low risk or high risk subset (Tier 4) based on a median metastatic risk score and calculating an metastatic risk score and stratifying the subject base on the metastatic risk score. In some examples, the method may include carrying out tier 6 analysis first and then subsequently carrying out tier 4 analysis.

In one example, the methods described herein include carrying out analysis according to Tier 5, and Tier 6. For example, the methods described herein may include stratifying the subject based on the mutational status of one or more additional Group A and/or Group B genomic aberrations to provide sub-risk groups (Tier 5) and calculating a metastatic risk score and stratifying the subject base on the metastatic risk score. In some examples, the method may include carrying out tier 5 analysis first and then subsequently carrying out tier 6 analysis. In some examples, the method may include carrying out tier 6 analysis first and then subsequently carrying out tier 5 analysis.

In one example, the methods described herein include carrying out analysis according to Tier 1, Tier 2, and Tier 3. For example, the methods described herein may include calculating a risk score of a subject as described herein stratifying the subject into one or more risk groups, stratifying the subject into one or more risk subgroups (Tier 2 groups) based on the mutational status of the representative Group A and/or Group B genomic aberrations and calculating an immune risk score and further stratifying the subject base on the immune risk score (tier 3). The tiers may be carried out in any order.

In one example, the methods described herein include carrying out analysis according to Tier 1, Tier 2, and Tier 4. For example, the methods described herein may include calculating a risk score of a subject as described herein, stratifying the subject into one or more risk groups, stratifying the subject into one or more risk subgroups (Tier 2 groups) based on the mutational status of the representative Group A and/or Group B genomic aberrations and stratifying the subject into an intermediate or high risk subset (Tier 4) based on a median risk score. The tiers may be carried out in any order with the proviso that for tier 4 analysis at least one of a risk score (tier 1) has been calculated prior to tier 4 analysis.

In one example, the methods described herein include carrying out analysis according to Tier 1, Tier 2, and Tier 5. For example, the methods described herein may include calculating a risk score of a subject as described herein, stratifying the subject into one or more risk groups, stratifying the subject into one or more risk subgroups (Tier 2 groups) based on the mutational status of the representative Group A and/or Group B genomic aberrations and stratifying the subject based on the mutational status of one or more additional Group A and/or Group B genomic aberrations to provide sub-risk groups (Tier 5). The tiers may be carried out in any order.

In one example, the methods described herein include carrying out analysis according to Tier 1, Tier 2, and Tier 6. For example, the methods described herein may include calculating a risk score of a subject as described herein, stratifying the subject into one or more risk groups, stratifying the subject into one or more risk subgroups (Tier 2 groups) based on the mutational status of the representative Group A and/or Group B genomic aberrations and calculating a metastatic risk score and stratifying the subject base on the metastatic risk score. The tiers may be caried out in any order.

In one example, the methods described herein include carrying out analysis according to Tier 1, Tier 3, and Tier 4. For example, the methods described herein may include calculating a risk score of a subject as described herein, stratifying the subject into one or more risk groups, calculating an immune risk score and further stratifying the subject based on the immune risk score (tier 3) and stratifying the subject into an intermediate or high risk subset (Tier 4) based on a median risk score and/or median immune score. The tiers may be carried out in any order with the proviso that for tier 4 analysis at least one of a risk score (tier 1), and/or immune risk score (tier 3) has been calculated prior to tier 4 analysis.

In one example, the methods described herein include carrying out analysis according to Tier 1, Tier 3, and Tier 5. For example, the methods described herein may include calculating a risk score of a subject as described herein, stratifying the subject into one or more risk groups, calculating an immune risk score and further stratifying the subject based on the immune risk score (tier 3) and stratifying the subject based on the mutational status of one or more additional Group A and/or Group B genomic aberrations to provide sub-risk groups (Tier 5). The tiers may be carried out in any order.

In one example, the methods described herein include carrying out analysis according to Tier 1, Tier 3, and Tier 6. For example, the methods described herein may include calculating a risk score of a subject as described herein, stratifying the subject into one or more risk groups, calculating an immune risk score and further stratifying the subject based on the immune risk score (tier 3) and calculating a metastatic risk score and stratifying the subject base on the metastatic risk score (tier 6).

The tiers may be carried out in any order.

In one example, the methods described herein include carrying out analysis according to Tier 1, Tier 4, and Tier 5. For example, the methods described herein may include calculating a risk score of a subject as described herein, stratifying the subject into one or more risk groups, further stratifying the subject into an intermediate or high risk subset (Tier 4) based on a median risk score and stratifying the subject based on the mutational status of one or more additional Group A and/or Group B genomic aberrations to provide sub-risk groups (Tier 5). The tiers may be carried out in any order with the proviso that for tier 4 analysis at least one of a risk score has been calculated prior to tier 4 analysis.

In one example, the methods described herein include carrying out analysis according to Tier 1, Tier 4, and Tier 6. For example, the methods described herein may include calculating a risk score of a subject as described herein, stratifying the subject into one or more risk groups, further stratifying the subject into an intermediate or high risk subset (Tier 4) based on a median risk score and/or median metastatic risk score and calculating a metastatic risk score and stratifying the subject base on the metastatic risk score (tier 6). The tiers may be carried out in any order with the proviso that for tier 4 analysis at least one of a risk score (tier 1), and/or metastatic risk score (tier 6) has been calculated prior to tier 4 analysis.

In one example, the methods described herein include carrying out analysis according to Tier 1, Tier 5, and Tier 6. For example, the methods described herein may include calculating a risk score of a subject as described herein, stratifying the subject into one or more risk groups, stratifying the subject based on the mutational status of one or more additional Group A and/or Group B genomic aberrations to provide sub-risk groups (Tier 5) and calculating a metastatic risk score and stratifying the subject base on the metastatic risk score (tier 6). The tiers may be carried out in any order.

In one example, the methods described herein include carrying out analysis according to Tier 2, Tier 3, and Tier 4. For example, the methods described herein may include stratifying the subject into one or more risk subgroups (Tier 2 groups) based on the mutational status of the representative Group A and/or Group B genomic aberrations and calculating an immune risk score and further stratifying the subject base on the immune risk score (tier 3), and further stratifying the subject into an intermediate or high risk subset (Tier 4) based on a median immune risk score. The tiers may be carried out in any order with the proviso that for tier 4 analysis at least an immune risk score (tier 3) has been calculated prior to tier 4 analysis.

In one example, the methods described herein include carrying out analysis according to Tier 2, Tier 3, and Tier 5. For example, the methods described herein may include stratifying the subject into one or more risk subgroups (Tier 2 groups) based on the mutational status of the representative Group A and/or Group B genomic aberrations and calculating an immune risk score and further stratifying the subject base on the immune risk score (tier 3) and stratifying the subject based on the mutational status of one or more additional Group A and/or Group B genomic aberrations to provide sub-risk groups (Tier 5). The tiers may be carried out in any order with the proviso that for tier 4 analysis at least one an immune risk score (tier 3) has been calculated prior to tier 4 analysis.

In one example, the methods described herein include carrying out analysis according to Tier 2, Tier 3, and Tier 6. For example, the methods described herein may include stratifying the subject into one or more risk subgroups (Tier 2 groups) based on the mutational status of the representative Group A and/or Group B genomic aberrations and calculating an immune risk score and further stratifying the subject base on the immune risk score (tier 3) and calculating an metastatic risk score and stratifying the subject base on the metastatic risk score (tier 6). The tiers may be caried out in any order.

In one example, the methods described herein include carrying out analysis according to Tier 2, Tier 4, and Tier 5. For example, the methods described herein may include stratifying the subject into one or more risk subgroups (Tier 2 groups) based on the mutational status of the representative Group A and/or Group B genomic aberrations, further stratifying the subject into a low risk or high risk subset (Tier 4) based on a median risk score and stratifying the subject based on the mutational status of one or more additional Group A and/or Group B genomic aberrations to provide sub-risk groups (Tier 5).

In one example, the methods described herein include carrying out analysis according to Tier 2, Tier 6, and Tier 4. For example, the methods described herein may include stratifying the subject into one or more risk subgroups (Tier 2 groups) based on the mutational status of the representative Group A and/or Group B genomic aberrations, calculating a metastatic risk score and stratifying the subject base on the metastatic risk score (tier 6) and further stratifying the subject into an intermediate or high risk subset (Tier 4) based on a median metastatic risk score. The tiers may be carried out in any order with the proviso that for tier 4 analysis at least a metastatic risk score (tier 6) has been calculated prior to tier 4 analysis.

In one example, the methods described herein include carrying out analysis according to Tier 2, Tier 5, and Tier 6. For example, the methods described herein may include stratifying the subject into one or more risk subgroups (Tier 2 groups) based on the mutational status of the representative Group A and/or Group B genomic aberrations, stratifying the subject based on the mutational status of one or more additional Group A and/or Group B genomic aberrations to provide sub-risk groups (Tier 5) and calculating a metastatic risk score and stratifying the subject base on the metastatic risk score (tier 6). The tiers may be carried out in any order.

In one example, the methods described herein include carrying out analysis according to Tier 3, Tier 4, and Tier 5. For example, the methods described herein may include calculating an immune risk score and further stratifying the subject base on the immune risk score, further stratifying the subject into an intermediate or high risk subset (Tier 4) based on a median immune risk score and, and stratifying the subject based on the mutational status of one or more additional Group A and/or Group B genomic aberrations to provide sub-risk groups (Tier 5). The tiers may be carried out in any order with the proviso that for tier 4 analysis at least an immune risk score (tier 3) has been calculated prior to tier 4 analysis.

In one example, the methods described herein include carrying out analysis according to Tier 3, Tier 6, and Tier 4. For example, the methods described herein may include calculating an immune risk score and further stratifying the subject base on the immune risk score, calculating a metastatic risk score and stratifying the subject base on the metastatic risk score (tier 6) and further stratifying the subject into an intermediate or high risk subset (Tier 4) based on a median immune risk score and/or metastatic risk score. The tiers may be carried out in any order with the proviso that for tier 4 analysis at least one of an immune risk score (tier 3) and/or metastatic risk score (tier 6) has been calculated prior to tier 4 analysis.

In one example, the methods described herein include carrying out analysis according to Tier 3, Tier 5, and Tier 6. For example, the methods described herein may include calculating an immune risk score and further stratifying the subject based on the immune risk score, stratifying the subject based on the mutational status of one or more additional Group A and/or Group B genomic aberrations to provide sub-risk groups (Tier 5) and calculating a metastatic risk score and stratifying the subject base on the metastatic risk score (tier 6). The tiers may be carried out in any order.

In one example, the methods described herein include carrying out analysis according to Tier 6, Tier 4, and Tier 5. For example, the methods described herein may include stratifying the subject baser on the metastatic risk score (tier 6), stratifying the subject into an intermediate or high risk subset (Tier 4) based on a median metastatic risk score and calculating a metastatic risk score and stratifying the subject based on the mutational status of one or more additional Group A and/or Group B genomic aberrations to provide sub-risk groups (Tier 5). The tiers may be carried out in any order with the proviso that for tier 4 analysis at least a metastatic risk score has been calculated prior to tier 4 analysis.

In one example, the methods described herein include carrying out analysis according to Tier 1, Tier 2, Tier 3, and Tier 4. For example, the methods described herein may include calculating a risk score of a subject as described herein, stratifying the subject into one or more risk groups, stratifying the subject into one or more risk subgroups (Tier 2 groups) based on the mutational status of the representative Group A and/or Group B genomic aberrations, calculating an immune risk score and further stratifying the subject base on the immune risk score (tier 3) and stratifying the subject into an intermediate or high risk subset (Tier 4) based on a median risk score and/or median immune risk score. The tiers may be carried out in any order with the proviso that for tier 4 analysis at least a risk score (tier 1) has been calculated prior to tier 4 analysis.

In one example, the methods described herein include carrying out analysis according to Tier 1, Tier 2, Tier 3, and Tier 5. For example, the methods described herein may include calculating a risk score of a subject as described herein, stratifying the subject into one or more risk groups, stratifying the subject into one or more risk subgroups (Tier 2 groups) based on the mutational status of the representative Group A and/or Group B genomic aberrations, calculating an immune risk score and further stratifying the subject base on the immune risk score (tier 3) and stratifying the subject based on the mutational status of one or more additional Group A and/or Group B genomic aberrations to provide sub-risk groups (Tier 5). The tiers may be carried out in any order.

In one example, the methods described herein include carrying out analysis according to Tier 1, Tier 2, Tier 3, and Tier 6. For example, the methods described herein may include calculating a risk score of a subject as described herein, stratifying the subject into one or more risk groups, stratifying the subject into one or more risk subgroups (Tier 2 groups) based on the mutational status of the representative Group A and/or Group B genomic aberrations, calculating an immune risk score and further stratifying the subject base on the immune risk score (tier 3) and calculating a metastatic risk score and stratifying the subject base on the metastatic risk score. The tiers may be carried out in any order.

In one example, the methods described herein include carrying out analysis according to Tier 1, Tier 2, Tier 4, and Tier 5. For example, the methods described herein may include calculating a risk score of a subject as described herein, stratifying the subject into one or more risk groups, stratifying the subject into one or more risk subgroups (Tier 2 groups) based on the mutational status of the representative Group A and/or Group B genomic aberrations, stratifying the subject into an intermediate or high risk subset (Tier 4) based on a median risk score and/or median immune risk score and stratifying the subject based on the mutational status of one or more additional Group A and/or Group B genomic aberrations to provide sub-risk groups (Tier 5). The tiers may be carried out in any order. The tiers may be carried out in any order with the proviso that for tier 4 analysis at least a risk score (tier 1) has been calculated prior to tier 4 analysis.

In one example, the methods described herein include carrying out analysis according to Tier 1, Tier 2, Tier 4, and Tier 6. For example, the methods described herein may include calculating a risk score of a subject as described herein, stratifying the subject into one or more risk groups, stratifying the subject into one or more risk subgroups (Tier 2 groups) based on the mutational status of the representative Group A and/or Group B genomic aberrations, stratifying the subject into an intermediate or high risk subset (Tier 4) based on a median risk score and/or median immune risk score and calculating an metastatic risk score and stratifying the subject base on the metastatic risk score. The tiers may be caried out in any order with the proviso that for tier 4 analysis at least a risk score (tier 1) has been calculated prior to tier 4 analysis.

In one example, the methods described herein include carrying out analysis according to Tier 1, Tier 2, Tier 5, and Tier 6. For example, the methods described herein may include calculating a risk score of a subject as described herein, stratifying the subject into one or more risk groups, stratifying the subject into one or more risk subgroups (Tier 2 groups) based on the mutational status of the representative Group A and/or Group B genomic aberrations, stratifying the subject based on the mutational status of one or more additional Group A and/or Group B genomic aberrations to provide sub-risk groups (Tier 5) and calculating an metastatic risk score and stratifying the subject base on the metastatic risk score. The tiers may be carried out in any order. The tiers may be carried out in any order.

In one example, the methods described herein include carrying out analysis according to Tier 1, Tier 3, Tier 4, and Tier 5. For example, the methods described herein may include calculating a risk score of a subject as described herein, stratifying the subject into one or more risk groups, calculating an immune risk score and further stratifying the subject base on the immune risk score (tier 3), stratifying the subject into an intermediate or high risk subset (Tier 4) based on a median risk score and/or median immune score and stratifying the subject based on the mutational status of one or more additional Group A and/or Group B genomic aberrations to provide sub-risk groups (Tier 5).

The tiers may be carried out in any order. The tiers may be carried out in any order with the proviso that for tier 4 analysis at least one of a risk score (tier 1) and/or an immune risk score (tier 3) has been calculated prior to tier 4 analysis.

In one example, the methods described herein include carrying out analysis according to Tier 1, Tier 3, Tier 4, and Tier 6. For example, the methods described herein may include calculating a risk score of a subject as described herein, stratifying the subject into one or more risk groups, calculating an immune risk score and further stratifying the subject base on the immune risk score (tier 3), stratifying the subject into an intermediate or high risk subset (Tier 4) based on a median risk score immune risk score and/or metastatic risk score and calculating a metastatic risk score and stratifying the subject base on the metastatic risk score. The tiers may be carried out in any order with the proviso that for tier 4 analysis at least one of a risk score (tier 1) and/or an immune risk score (tier 3) has been calculated prior to tier 4 analysis.

In one example, the methods described herein include carrying out analysis according to Tier 1, Tier 3, Tier 5, and Tier 6. For example, the methods described herein may include calculating a risk score of a subject as described herein, stratifying the subject into one or more risk groups, calculating an immune risk score and further stratifying the subject based on the immune risk score (tier 3), stratifying the subject based on the mutational status of one or more additional Group A and/or Group B genomic aberrations to provide sub-risk groups (Tier 5) and calculating an metastatic risk score and stratifying the subject base on the metastatic risk score. The tiers may be carried out in any order.

In one example, the methods described herein include carrying out analysis according to Tier 1, Tier 4, Tier 5, and Tier 6. For example, the methods described herein may include calculating a risk score of a subject as described herein, stratifying the subject into one or more risk groups, further stratifying the subject into an intermediate or high risk subset (Tier 4) based on a median risk score, and/or metastatic risk score, stratifying the subject based on the mutational status of one or more additional Group A and/or Group B genomic aberrations to provide sub-risk groups (Tier 5) and calculating a metastatic risk score and stratifying the subject base on the metastatic risk score. The tiers may be carried out in any order with the proviso that for tier 4 analysis at least one of a risk score (tier 1) and/or a metastatic risk score (tier 6) has been calculated prior to tier 4 analysis.

In one example, the methods described herein include carrying out analysis according to Tier 2, Tier 3, Tier 4, and Tier 5. For example, the methods described herein may include stratifying the subject into one or more risk subgroups (Tier 2 groups) based on the mutational status of the representative Group A and/or Group B genomic aberrations and calculating an immune risk score and further stratifying the subject base on the immune risk score (tier 3), further stratifying the subject into an intermediate or high risk subset (Tier 4) based on a median risk score and/or immune risk score and stratifying the subject based on the mutational status of one or more additional Group A and/or Group B genomic aberrations to provide sub-risk groups (Tier 5). The tiers may be carried out in any order with the proviso that for tier 4 analysis at least an immune risk score (tier 3) has been calculated prior to tier 4 analysis.

In one example, the methods described herein include carrying out analysis according to Tier 2, Tier 3, Tier 4, and Tier 6. For example, the methods described herein may include stratifying the subject into one or more risk subgroups (Tier 2 groups) based on the mutational status of the representative Group A and/or Group B genomic aberrations and calculating an immune risk score and further stratifying the subject based on the immune risk score (tier 3), further stratifying the subject into an intermediate or high risk subset (Tier 4) based on a median risk score, immune risk score and/or metastatic risk score and calculating an metastatic risk score and stratifying the subject base on the metastatic risk score. The tiers may be carried out in any order with the proviso that for tier 4 analysis at least one of a metastatic risk score (tier 6) and/or an immune risk score (tier 3) has been calculated prior to tier 4 analysis.

In one example, the methods described herein include carrying out analysis according to Tier 2, Tier 3, Tier 5, and Tier 6. For example, the methods described herein may include stratifying the subject into one or more risk subgroups (Tier 2 groups) based on the mutational status of the representative Group A and/or Group B genomic aberrations and calculating an immune risk score and further stratifying the subject base on the immune risk score (tier 3), stratifying the subject based on the mutational status of one or more additional Group A and/or Group B genomic aberrations to provide sub-risk groups (Tier 5) and calculating an metastatic risk score and stratifying the subject based on the metastatic risk score. The tiers may be carried out in any order.

In one example, the methods described herein include carrying out analysis according to Tier 2, Tier 4, Tier 5, and Tier 6. For example, the methods described herein may include stratifying the subject into one or more risk subgroups (Tier 2 groups) based on the mutational status of the representative Group A and/or Group B genomic aberrations and further stratifying the subject into an intermediate or high risk subset (Tier 4) based on a median risk score, and/or metastatic risk score, stratifying the subject based on the mutational status of one or more additional Group A and/or Group B genomic aberrations to provide sub-risk groups (Tier 5) and calculating a metastatic risk score and stratifying the subject base on the metastatic risk score. The tiers may be carried out in any order with the proviso that for tier 4 analysis at least one of a metastatic risk score (tier 6) has been calculated prior to tier 4 analysis.

In one example, the methods described herein include carrying out analysis according to Tier 3, Tier 4, Tier 5, and Tier 6. For example, the methods described herein may include calculating an immune risk score and further stratifying the subject based on the immune risk score, further stratifying the subject into an intermediate or high risk subset (Tier 4) based on a median immune risk score, stratifying the subject based on the mutational status of one or more additional Group A and/or Group B genomic aberrations to provide sub-risk groups (Tier 5) and calculating an metastatic risk score and stratifying the subject base on the metastatic risk score. The tiers may be carried out in any order with the proviso that for tier 4 analysis at least one of a metastatic risk score (tier 1) and/or an immune risk score (tier 3) has been calculated prior to tier 4 analysis.

In one example, the methods described herein include carrying out analysis according to Tier 1, Tier 2, Tier 3, Tier 4, and Tier 5. For example, the methods described herein may include calculating a risk score of a subject as described herein, stratifying the subject into one or more risk groups (tier 1), stratifying the subject into one or more risk subgroups (Tier 2 groups) based on the mutational status of the representative Group A and/or Group B genomic aberrations, calculating an immune risk score and further stratifying the subject base on the immune risk score (tier 3), stratifying the subject into an intermediate or high risk subset (Tier 4) based on a median risk score and/or median immune risk score and stratifying the subject based on the mutational status of one or more additional Group A and/or Group B genomic aberrations to provide sub-risk groups (Tier 5). The tiers may be carried out in any order with the proviso that for tier 4 analysis at least one of a risk score (tier 1), and/or immune risk score (tier 3) has been calculated prior to tier 4 analysis.

In one example, the methods described herein include carrying out analysis according to Tier 1, Tier 2, Tier 3, Tier 4, and Tier 6. For example, the methods described herein may include calculating a risk score of a subject as described herein stratifying the subject into one or more risk groups (tier 1), stratifying the subject into one or more risk subgroups (Tier 2 groups) based on the mutational status of the representative Group A and/or Group B genomic aberrations, calculating an immune risk score and further stratifying the subject base on the immune risk score (tier 3), stratifying the subject into an intermediate or high risk subset (Tier 4) based on a median risk score and/or median immune risk score and calculating an metastatic risk score and stratifying the subject base on the metastatic risk score. The tiers may be caried out in any order with the proviso that for tier 4 analysis at least one of a risk score (tier 1), immune risk score (tier 3) and/or metastatic risk score (tier 6) has been calculated prior to tier 4 analysis.

In one example, the methods described herein include carrying out analysis according to Tier 1, Tier 2, Tier 3, Tier 5, and Tier 6. For example, the methods described herein may include calculating a risk score of a subject as described herein stratifying the subject into one or more risk groups (tier 1), stratifying the subject into one or more risk subgroups (Tier 2 groups) based on the mutational status of the representative Group A and/or Group B genomic aberrations, calculating an immune risk score and further stratifying the subject based on the immune risk score (tier 3), stratifying the subject based on the mutational status of one or more additional Group A and/or Group B genomic aberrations to provide sub-risk groups (Tier 5) and calculating a metastatic risk score and stratifying the subject base on the metastatic risk score. The tiers may be carried out in any order.

In one example, the methods described herein include carrying out analysis according to Tier 1, Tier 2, Tier 4, Tier 5, and Tier 6. For example, the methods described herein may include calculating a risk score of a subject as described herein, stratifying the subject into one or more risk groups (tier 1), stratifying the subject into one or more risk subgroups (Tier 2 groups) based on the mutational status of the representative Group A and/or Group B genomic aberrations, stratifying the subject into an intermediate or high risk subset (Tier 4) based on a median risk score and/or median immune risk score stratifying the subject based on the mutational status of one or more additional Group A and/or Group B genomic aberrations to provide sub-risk groups (Tier 5) and calculating a metastatic risk score and stratifying the subject base on the metastatic risk score. The tiers may be caried out in any order with the proviso that for tier 4 analysis at least one of a risk score (tier 1) and/or metastatic risk score (tier 6) has been calculated prior to tier 4 analysis.

In one example, the methods described herein include carrying out analysis according to Tier 1, Tier 3, Tier 4, Tier 5, and Tier 6. For example, the methods described herein may include calculating a risk score of a subject as described herein, stratifying the subject into one or more risk groups, calculating an immune risk score and further stratifying the subject based on the immune risk score (tier 3), stratifying the subject into an intermediate or high risk subset (Tier 4) based on a median risk score and/or median immune score, stratifying the subject based on the mutational status of one or more additional Group A and/or Group B genomic aberrations to provide sub-risk groups (Tier 5) and calculating a metastatic risk score and stratifying the subject base on the metastatic risk score. The tiers may be carried out in any order with the proviso that for tier 4 analysis at least one of a risk score (tier 1), immune risk score (tier 3) and/or metastatic risk score (tier 6) has been calculated prior to tier 4 analysis.

In one example, the methods described herein include carrying out analysis according to Tier 2, Tier 3, Tier 4, Tier 5, and Tier 6. For example, the methods described herein may include stratifying the subject into one or more risk subgroups (Tier 2 groups) based on the mutational status of the representative Group A and/or Group B genomic aberrations and calculating an immune risk score and further stratifying the subject base on the immune risk score (tier 3), further stratifying the subject into an intermediate or high risk subset (Tier 4) based on a median risk score and/or immune risk score, stratifying the subject based on the mutational status of one or more additional Group A and/or Group B genomic aberrations to provide sub-risk groups (Tier 5) and calculating an metastatic risk score and stratifying the subject base on the metastatic risk score. The tiers may be carried out in any order with the proviso that for tier 4 analysis at least one of an immune risk score (tier 3) and/or metastatic risk score (tier 6) has been calculated prior to tier 4 analysis.

In one example, the methods described herein include carrying out analysis according to Tier 1, Tier 2, Tier 3, Tier 4, Tier 5, and Tier 6. For example, the methods described herein may include calculating a risk score of a subject as described herein, stratifying the subject into one or more risk groups, stratifying the subject into one or more risk subgroups (Tier 2 groups) based on the mutational status of the representative Group A and/or Group B genomic aberrations and calculating an immune risk score and further stratifying the subject base on the immune risk score (tier 3), further stratifying the subject into an intermediate or high risk subset (Tier 4) based on a median risk score and/or immune risk score, stratifying the subject based on the mutational status of one or more additional Group A and/or Group B genomic aberrations to provide sub-risk groups (Tier 5) and calculating a metastatic risk score and stratifying the subject based on the metastatic risk score. The tiers may be carried out in any order with the proviso that for tier 4 analysis at least one of a risk score (tier 1), immune risk score (tier 3) and/or metastatic risk score (tier 6) has been calculated prior to tier 4 analysis.

### Histopathological Classification

In some examples, subjects and the groups, subgroups, immune subsets, metastatic subsets and/or sub-risk sets may also be stratified according to known classifications used in the field.

For example, for cancer, the groups of tiers 1 to 6 may be further stratified by histopathological methods. Histopathological classification or stratification may be done before, concurrently, and/or after determining the groups of each tier as described herein.

In some examples, when the disease breast cancer, one or more of the hormone receptor ER (estrogen receptor, ESR1), PR (progesterone receptor, PGR), and/or HER2 status of a breast tumour sample are determined and used to further stratify a subject and the plurality of control subjects.

In some examples, the ER, PR, and/or HER2 status are determined or known before determining a tier group and/or immune risk score and/or metastatic risk score of a subject. In other examples, the ER, PR, and HER2 status is determined concurrently with a tier group and/or immune risk score and/or metastatic risk score of a subject.

ER, PR and/or HER2 status in some examples are determined at the nucleic acid level using known methods in field such as nucleic acid methods described herein (e.g., by microarray). In other examples, ER, PR and/or HER2 status is determined at the protein level (e.g., by immunochemistry methods known in the art).

In some examples, when the disease is breast cancer the lymph nodal status (lymph node-positive or lymph node-negative), of a subject is determined before or concurrently with determination of a tier group and/or immune risk score and/or metastatic risk score of a subject or after determining a tier group and/or immune risk score and/or metastatic risk score of a subject.

When the disease is prostate cancer, the Gleason grade or the lymph nodal status (lymph node-positive or lymph node-negative) may be determined before or concurrently with the determination of a tier group and/or immune risk score of a subject or after determining a tier group and/or immune risk score and/or metastatic risk score of a subject.

When the disease is endometrial cancer, the tumour type (Uterine Endometrioid Carcinoma or Uterine Serous Carcinoma) or the lymph nodal status (lymph node-positive or lymph node-negative) may be determined before or concurrently with the determination of a tier group and/or immune risk score and/or metastatic risk score of a subject or after determining a tier group and/or immune risk score and/or metastatic risk score of a subject.

### Additional Analysis and Identification of Biomarkers

The methods provided herein may also allow for detection of specific target genes and/or pathways that may be targeted to treat the disease and specific subject groups.

For example, the method may further include the use of proteomic analysis to provide further insight into specific groups defined in each tier. For example by protein detection methods including but not limited to, RPPA, immunohistochemistry, ELISA, suspension bead array, mass spectrometry, dot blot, or western blot analysis.

In some examples, reverse-phase protein arrays (RPPA) may be used to determine underlying mechanisms involved in the pathology of each group. Reverse phase protein (micro)-array (RPPA) is a sensitive, high throughput, functional proteomic technology that offers many advantages.

It extends the power of immunoblotting to provide a quantitative analysis of the differential expression of active (usually phosphorylated or cleaved) and parental proteins. Proteins and their corresponding phosphoproteins can be assessed reflecting the activation state/functionality of a given protein.

Such analysis may be carried out on the plurality of control subjects in each group to identify levels of protein expression that differs between groups. By identifying proteins that are differentially expressed between two groups, such as each tier 2 risk subgroup, proteins that may be targeted in that group may be identified. With RPPA all samples are spotted at the same time making this method ideally suited for retrospective analysis of large numbers of specimens similar to the idea of gene microarrays. Compared to a conventional Western blotting, which uses protein from 5×10⁵ cells, RPPA requires nanolitres of protein lysate (pico- to femtograms of protein). Protein equivalent to 200 cells is printed per slide, per single antibody. Thus samples prepared from only 5,000-20,000 cells are sufficient to analyse 100 different protein targets and from the material previously required for a single western blot, 2500 slides (theoretically=2500 antibodies) can be printed. The printing precision and reliability of RPPA technology are extremely high with low experimental variability.

In some examples, statistical based methods such as Gene Set Enrichment analysis (GSEA), over-representation analysis, pathway enrichment analysis, and/or network enrichment analysis may be used to identify pathways and/or genes that may be targeted in specific groups.

Gene Set Enrichment Analysis (GSEA) is a computational method that determines whether an a priori defined set of genes shows statistically significant, concordant differences between two biological states (e.g. phenotypes). Details of GSEA can be found in Subramanian, Tamayo, et al. (2005, PNAS) and Mootha, Lindgren, et al. (2003, Nature Genetics) which is incorporated herein in its entirety. As used herein, GSEA refers to a method to identify up-regulated gene sets and molecular pathway activities within clusters that are established based on quantitative PWI features. GSEA attributes a specific weight to each gene/protein in the input list that depends on a metric of choice, which is usually represented by quantitative expression data. GSEA can provide insight into each group by focusing on gene sets, that is, groups of genes that share a common biological function, chromosomal location, or regulation. For example, overrepresentation (or enrichment) analysis is a statistical method that determines.

Expression data for the plurality of control subjects in each group may be provided in the data or may be determined using methods as described herein. By carrying out GSEA analysis on each group in each tier, upregulated and/or downregulated genes that differ between each group may be identified and thus may provide a group specific target or biomarker that's modulation may provide a more targeted and subject specific treatment plan.

Over-representation (or enrichment) analysis is a statistical method that determines whether genes from pre-defined sets (for example, those belonging to a specific GO term, KEGG, Reactome, PANTHER or other pathway) are present more than would be expected (over-represented) in a subset of data, for example the data for the plurality of control subjects in each tier group. Such analysis may be carried out using software such as ClusterProfiler®.

Other suitable software for arraying out pathway and/or network enrichment analysis include g:Profiler, ExpressAnalyst, Database for Annotation, Visualization and Integrated Discovery (DAVID), Cytoscape and EnrichmentMap.

Given the above, there is also provided herein a method of identifying one or more biomarkers for a disease comprising one or more genomic aberrations, the method comprising:
a. analysing expression data of a plurality of subjects suffering from disease, wherein each subject has been stratified into one or more groups as described herein; and
b. identifying one or more genes and/or proteins differentially expressed between at least two groups as described herein.

As described above, the plurality of subjects may be the plurality of control subjects as described herein. In some examples, the disease is cancer such as breast cancer, prostate cancer or endometrial cancer. In some examples, identifying may comprise using one or more of Reverse phase protein array (RPPA) analysis, Gene Set Enrichment analysis (GSEA), over-representation analysis, pathway enrichment analysis, and/or network enrichment analysis.

An alternative way to stratify subjects into respective tier-1, tier-2, tier-3, tier-4, tier-5 and/or tier-6 molecular subgroups, is using proteogenomic characterisation and protein markers to discriminate between molecular groups (i.e. as an alternative way to stratify tumours into Tier 1, Tier 2, 3, 4, 5 or 6 subgroups or subsets). For example, some proteins are highly enriched in various breast cancer tier-2 molecular groups: XRCC1 and Cyclin D1 for Subgroup 2, Akt_pT308 and Tuburin_pT1462 for Subgroup 3, mTOR and cell cycle protein gene sets (e.g., S6, 4EBP1_pS65, FoxM1, Cyclin B1, etc.) for Subgroup 4 and G6PD for Subgroup 5. This shows the potential for molecular group classifications to be adopted in conventional pathology laboratories using immunohistochemistry approaches.

Similarly, other omics-based methods such as metabolomics and/or epigenetics (e.g. methylation and/or acetylation) could be used to detect biomarkers (group-specific biomarkers) for stratifying into various groups, subgroups and/or subsets as described herein (after characterising the various molecular subgroups identified here using omics-based approaches) as an alternative way to stratify a disease, for example, a tumour or subject suffering therefrom into respective tier-1, 2, 3, 4, 5 and/or 6 subgroups as described herein.

Similarly, molecular subgroup-specific distinct gene signatures could be derived by comparing subgroups with each other using various statistical approaches described above and identifying a unique set of genes that define each molecular subgroup. These gene signatures highlight an alternative way to stratify a disease, for example, a tumour or subject suffering therefrom into respective tier-1, 2, 3, 4, 5 and/or 6 subgroups as described herein.

As such, there is provided a method of determining or predicting a subject's prognosis by comparing levels of the group specific markers in a subject sample to a predetermined (e.g. a model or database) set of one or more group-specific biomarkers. For example, if the subject has comparable levels of the group-specific biomarkers as those form a control sample, the subject may be classified or stratified into the corresponding group or subgroup.

### Prognosis

The methods provided above can be used to stratify a subject into certain groups. Each of these groups may be used to provide a prognosis of a subject in the specified group. As such, the methods described herein may be used for determining and/or predicting the prognosis of a subject.

In one examples, there is provided method of determining a prognosis for a subject suffering from a disease, the disease comprising one or more genomic aberrations, the method comprising: providing a subject sample; identifying one or more genomic aberrations associated with the disease from the subject sample; classifying the one or more genomic aberrations and stratifying the subject as described herein; and determining a prognosis for the subject based on the risk score. The method may include stratifying the subject based on analysis as described for any one of tiers 2, 3, 4, 5, and/or 6 as described herein. For example, the method may further include, calculating an immune risk as described herein and further stratifying the subject based on the immune risk score and/or calculating a metastatic risk score as described herein further stratifying the subject based on the metastatic risk score.

In another example, there is provided a method of determining a prognosis for a subject suffering from a disease, the disease comprising one or more genomic aberrations, the method comprising: providing a subject sample; analysing the subject sample and calculating an immune risk score for the subject and stratifying the subject as described herein; and determining a prognosis for the subject based on the immune risk score. The method may include stratifying the subject based on analysis as described for any one of tiers 1, 2, 4, 5, and/or 6 as described herein. For example, the method may further include, calculating a risk score as described herein and further stratifying the subject based on the risk score and/or calculating a metastatic risk score as described herein further stratifying the subject based on the metastatic risk score.

In another example, there is provided a method of determining a prognosis for a subject suffering from a disease, the disease comprising one or more genomic aberrations, the method comprising: providing a subject sample; analysing the subject sample and calculating a metastatic risk score for the subject and stratifying the subject as described herein; and determining a prognosis for the subject based on the metastatic risk score. The method may include stratifying the subject based on analysis as described for any one of tiers 1, 2, 3, 4, and/or 5, as described herein. For example, the method may further include, calculating a risk score as described herein and further stratifying the subject based on the risk score and/or calculating an immune risk score as described herein further stratifying the subject based on the immune risk score.

In another example, there is provided a method of determining a prognosis for a subject suffering from a disease, the disease comprising one or more genomic aberrations, the method comprising: providing a subject sample and carrying out tier 2 analysis as descried herein to stratify the subject. The method may include stratifying the subject based on analysis as described for any one of tiers 1, 3, 4, 5, and/or 6, as described herein. In another example, there is provided a method of determining a prognosis for a subject suffering from a disease, the disease comprising one or more genomic aberrations, the method comprising: providing a subject sample and carrying out tier 5 analysis as descried herein to stratify the subject. The method may include stratifying the subject based on analysis as described for any one of tiers 1, 2, 3, 4, and/or 6, as described herein.

Determining prognosis” or “predicting prognosis” refers to methods which can predict the course or outcome of a disease in a subject. The term “prognosis” does not refer to the ability to predict the course or outcome of a condition with 100% accuracy, or even that a given course or outcome is predictably more or less likely to occur based on the risk score, immune risk score and/or metastatic risk score as described herein. Instead, it will be understood that the term “prognosis” refers to an increased probability that a certain course or outcome will occur; that is, that a course or outcome is more likely to occur in a subject suffering from a given disease including the analysed genomic aberrations, when compared to those individuals not suffering from the disease and/or subject does not include the respective genomic aberration i.e. is wild-type in respect of the respective genomic aberration. For example, in subjects not suffering from the disease (e.g., not having one or more of the genomic aberrations as described herein), the chance of a given outcome (e.g., suffering from relapse of cancer) may be very low.

Prognosis may include any one or more of likelihood of relapse, time to relapse, overall survival rate, disease-free survival, recurrence-free survival, metastasis-free survival, event-free survival, time to metastasis, likelihood of metastasis, and/or efficacy of a treatment.

Prognosis may include the likelihood of relapse of subject. The term “relapse” refers to the diagnosis of return, or signs and symptoms of return, of a disease such as cancer after a period of improvement or remission. In the case of cancer “Relapse” can also include “recurrence,” which the National Cancer institute defines as cancer that has recurred, usually after a period of time during which the cancer could not be detected. The cancer may come back to the same location in the body as the original (primary) tumour or to another location in the body (NCI Dictionary of Cancer Terms).

In some examples, not detecting a cancer can include not detecting cancer cells in the subject, not detecting tumours in the subject, and/or no symptoms, in whole or in part, associated with the cancer.

“Overall survival rate” refers to the percentage of people in a study or treatment group who are still alive for a certain period of time after they were diagnosed with or started treatment for a disease. For example, overall survival rate may be a percentage of subjects still alive after a period of 2 years, 5 years, 10 years or 20 years.

“Disease free survival” refers to the length of time after primary treatment for a cancer ends that the patient survives without any signs or symptoms of that cancer.

The prognosis of a subject may be based on a prognosis determined for the plurality of subjects that fall within the same tier group. For example, by analysing the prognosis of each of the plurality of control subjects in each tier group or subgroup, a prognosis can be assigned to the subgroup defined by the score of those subjects with the group or subgroup.

In order to determine a prognosis for a tier group methods such as Kaplan-Meier methods may be used. A Kaplan-Meier survival curve is defined as the probability of surviving in a given length of time while considering time in many small intervals. There are three assumptions used in this analysis. Firstly, it is assumed that at any time subjects who are censored have the same survival prospects as those who continue to be followed. Secondly, it is assumed that the survival probabilities are the same for subjects recruited early and late in the study. Thirdly, it is assumed that the event happens at the time specified. The Kaplan-Meier estimate is also called as “product limit estimate”. It involves computing of probabilities of occurrence of an event at a certain point of time. These successive probabilities are multiplied by any earlier computed probabilities to get the final estimate. total probability of survival till that time interval is calculated by multiplying all the probabilities of survival at all time intervals preceding that time (by applying law of multiplication of probability to calculate cumulative probability). For example, the probability of a patient surviving two days after a kidney transplant can be considered to be probability of surviving the one day multiplied by the probability surviving the second day given that patient survived the first day. This second probability is called as a conditional probability. For more details of Kaplan-Meier analysis see Goel M K, Khanna P, Kishore J. Understanding survival analysis: Kaplan-Meier estimate. Int J Ayurveda Res. 2010; 1(4):274-278. doi:10.4103/0974-7788.76794 which is incorporated herein in its entirety.

Other analysis methods may be used such as Cox-regression methods as described in Abd ElHafeez, Samar, et al. “Methods to analyze time-to-event data: the Cox regression analysis.” Oxidative Medicine and Cellular Longevity 2021 (2021) which is incorporated herein by reference in its entirety.

### Diseases

The methods described herein have been described using cancer as an example disease. However, it will be apparent that the methods described herein can be applied to any disease that includes one or more genomic aberrations as described herein. That is to say that any disease that includes pathogenic gene mutations and/or genetic variations.

For example, the disease may be any one of genetic/inherited disorders, including PIK3CA-related overgrowth spectrum (PROS), PTEN Hamartoma Tumor Syndrome (PHTS—that includes clinical disorders: Cowden syndrome, Bannayan-Riley-Ruvalcaba syndrome, Proteus syndrome, and Proteus-like syndrome), Hereditary breast and ovarian cancer syndrome (HBOC), Lynch syndrome, Familial adenomatous polyposis (FAP), MUTYH-associated polyposis (MAP), Familial juvenile polyposis, Peutz Jeghers syndrome, Sotos syndrome, Neurofibromatosis 1 (NF1), Multiple endocrine neoplasia 2B (MEN 2B), Down Syndrome, Thalassemia, Cystic Fibrosis, Tay-Sachs disease, Sickle Cell Anaemia, and/or neurodegenerative disorders like Parkinson's disease and Alzheimer's disease, autism, and Huntington's disease and/or mTOR hyperactivation-associated syndromes or other rare syndromes.

In some examples, the methods and databases described herein may be used to identify targets in a subject suffering from an unrelated disease that includes at least one genomic aberration that is also included in the disease of the plurality of control subjects. For example, a subject may be suffering from autism and includes one or more PHTS-mutations. By using the methods and databases described biomarkers or targets that are linked to PTEN-mutations may be identified and thus also targeted in subjects suffering from autism.

### Treatment

The stratification of subjects using the methods described herein may provide a medical practitioner a diagnostic report based on the patient's stratification, prognosis and/or disease underlying biology. As such, the methods provided herein may further include generating a diagnostic report based on the subject's prognosis, underlying disease biology and/or stratification. In certain examples, the diagnostic report is provided to a medical professional (such as a medical doctor) for providing guidance on selection of a treatment to be administered.

In some examples, the methods further comprise administering to the subject a treatment.

In some examples, the methods further comprise administering to the subject a treatment regimen based on the patient's prognosis, stratification and/or underlying disease biology determined by the methods described herein.

The methods described herein can further comprise selecting, and optionally administering, a treatment regimen for the subject based on the prognosis, underlying disease biology or stratification (i.e., the tier subgroup described herein). Treatment can include, for example, surgery, therapy (e.g., radiation, hormone, ultrasound, chemotherapy, immunotherapy, targeted therapy), or combinations thereof. However, in some cases, immediate treatment may not be required, and the subject may be selected for active surveillance. The selection of a treatment or further treatment can be based on the risk score and/or immune risk score of a subject calculated as described herein. For example, the treatment may be selected depending on whether a subject is stratified as having greater than 90% survival over a time period such as 5 years, 8 years or 10 years or less than 90% survival of over a time period such as 5 years, 8 years or 10 years.

In some examples, subjects may not be provided a treatment based on the prognosis and/or stratification determined using the methods described herein. Thus avoiding over treatment (e.g. when treatment is not necessary). For example, if a subject is in a group or subgroup of one or more of the tiers described herein that has greater than 90% survival likelihood over a time period such as 5 years or 10 years treatment may not be needed and/or administered. In some examples, subject may be subjected to active surveillance or monitoring and/or surgery.

In some examples, treatment may be administered early to a subject being in a group or subgroup that has a negative or worse prognosis determined by the methods as described herein than a subject who is stratified into a group or subgroup with a better prognosis.

As such, there is provided herein a method of determining a treatment regimen for a subject based up stratification and/or prognosis of the subject using the methods as described herein.

As used herein, the terms “active surveillance”, “monitoring” and “watchful waiting” are used interchangeably herein to mean closely monitoring a subject's condition without giving any treatment until symptoms appear or change.

As used herein, the terms “treat”, “treating” and “treatment” are taken to include an intervention performed with the intention of preventing the development or altering the pathology of a condition, disorder or symptom. Accordingly, “treatment” refers to both therapeutic treatment and prophylactic or preventative measures, wherein the object is to prevent or slow down (lessen) the targeted condition, disorder or symptom. “Treatment” therefore encompasses a reduction, slowing or inhibition of the symptoms of a disease, such as cancer, for example of at least 5%, 10%, 20%, 30%, 40%, 50%, 60%, 70%, 80%, 90% or 100% when compared to the symptoms before treatment.

When a therapeutic agent or other treatment is administered, it is administered in an amount and/or for a duration that is effective to treat the disease or to reduce the likelihood (or risk) of the disease developing in the future. An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. For example, an effective amount can depend upon the degree to which a subject has abnormal levels of certain analytes that are indicative of the disease. It should be understood that any therapeutic agents described herein are used to treat and/or prevent diseases. Thus, in some cases, they may be used prophylactically in subjects at risk of developing the disease or who are at risk of relapse. Thus, in some cases, an effective amount is that amount which can lower the risk of, slow or perhaps prevent altogether the development of the disease. It will be recognized when the therapeutic agent is used in acute circumstances, it is used to prevent one or more medically undesirable results that typically flow from such adverse events. Methods for selecting a suitable treatment, an appropriate dose thereof and modes of administration will be apparent to one of ordinary skill in the art.

The medications or treatments described herein can be administered to the subject by any conventional route, including injection or by gradual infusion overtime. The administration may, for example, be by infusion or by intramuscular, intravascular, intracavity, intracerebral, intralesional, rectal, subcutaneous, intradermal, epidural, intrathecal, percutaneous administration. The medications may also be given in e.g. tablet form or in solution. Several appropriate medications and means for administration of the same are well known in the art.

Given the above, there is provided herein a method of treating a subject suffering from a disease comprising one or more genomic aberrations, the method comprising:
a. stratifying a subject as described herein;
b. determining a prognosis for the subject based on the stratification of the subject;
c. generating a diagnostic report based on the prognosis and/or underlying disease biology;
d. administering a treatment to the subject based on the prognosis and/or underlying disease biology.

There is also provided herein a treatment for use in a method of treating a subject suffering from a disease comprising one or more genomic aberrations, the method comprising:
a. stratifying a subject as described herein;
b. determining a prognosis for the subject based on the stratification of the subject;
c. generating a diagnostic report based on the prognosis and/or underlying disease biology;
d. administering a treatment to the subject based on the prognosis and/or underlying disease biology.

The treatment administered to a subject will be dependent on the disease and the stratification, underlying disease biology and/or prognosis of the subject as determined by the methods provided herein.

For example, if the disease is cancer the treatment may be selected from chemotherapy, hormone therapy, radiotherapy, immunotherapy, targeted therapy, surgery and/or wherein immediate therapeutic intervention may not be required, and the subject may be selected for active surveillance.

“Chemotherapy” refers to treatment with one or more therapeutic agents to reduce or eliminate the growth or proliferation of cancer cells.

Examples of therapeutic agents include, but are not limited to, antibodies, antibody fragments, conjugates, drugs, cytotoxic agents, proapoptotic agents, toxins, nucleases (including DNAses and RNAses), hormones, immunomodulators, chelators, boron compounds, photoactive agents or dyes, radioisotopes or radionuclides, oligonucleotides, interference RNA, peptides, anti-angiogenic agents, chemotherapeutic agents, cytokines, chemokines, prodrugs, enzymes, binding proteins or peptides or combinations thereof.

For example, chemotherapeutic drugs include vinca alkaloids, anthracyclines, epidophyllotoxins, taxanes, antimetabolites, tyrosine kinase inhibitors, alkylating agents, antibiotics, Cox-2 inhibitors, antimitotics, antiangiogenic and proapoptotic agents, doxorubicin, methotrexate, taxol, other camptothecins, and others from these and other classes of anticancer agents, and the like. Other cancer chemotherapeutic drugs include nitrogen mustards, alkyl sulfonates, nitrosoureas, triazenes, folic acid analogs, pyrimidine analogs, purine analogs, platinum coordination complexes, hormones, and the like. Suitable chemotherapeutic agents are described in REMINGTON'S PHARMACEUTICAL SCIENCES, 19th Ed. (Mack Publishing Co. 1995), and in GOODMAN AND GILMAN'S THE PHARMACOLOGICAL BASIS OF THERAPEUTICS, 7th Ed. (MacMillan Publishing Co. 1985), as well as revised editions of these publications. Other suitable chemotherapeutic agents, such as experimental drugs, are known to those of skill in the art.

Exemplary drugs include, but are not limited to, 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, Celebrex, chlorambucil, cisplatin (CDDP), Cox-2 inhibitors, irinotecan (CPT-1 1), SN-38, carboplatin, cladribine, camptothecans, crizotinib, cyclophosphamide, cytarabine, dacarbazine, dasatinib, dinaciclib, docetaxel, dactinomycin, daunorubicin, doxorubicin, 2-pyrrolinodoxorubicine (2P-DOX), cyano-morpholino doxorubicin, doxorubicin glucuronide, epirubicin glucuronide, erlotinib, estramustine, epidophyllotoxin, erlotinib, entinostat, estrogen receptor binding agents, etoposide (VP 16), etoposide glucuronide, etoposide phosphate, exemestane, fingolimod, floxuridine (FUdR), 3′, 5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU 11248, sunitinib, tamoxifen, temazolomide (an aqueous form of DTIC), transplatinum, thalidomide, thioguanine, thiotepa, teniposide, topotecan, uracil mustard, vatalanib, vinorelbine, vinblastine, vincristine, vinca alkaloids and ZD 1839.

“Radiation therapy” refers to a cancer treatment that uses high-energy x-rays or other types of radiation to kill cancer cells or keep them from growing. There are two types of radiation therapy. External radiation therapy uses a machine outside the body to send radiation toward the cancer. Certain ways of giving external radiation therapy can help keep radiation from damaging nearby healthy tissue. For example, three-dimensional conformal radiation therapy (3D-CRT) uses computers to precisely map the location of the tumour. Radiation beams are then shaped and aimed at it from several directions, which makes it less likely to damage normal tissues. Another example is, intensity-modulated radiation therapy (IMRT) is a type of 3-dimensional (3-D) radiation therapy that uses a computer to make pictures of the size and shape of the tumour. Thin beams of radiation of different intensities (strengths) are aimed at the tumour from many angles. Radiation therapy also includes proton beam radiation therapy, image guided radiation therapy (IGRT), helical-tomotherapy and photon beam radiation therapy.

Internal radiation therapy (also referred to as brachytherapy) uses a radioactive substance sealed in needles, seeds, wires, or catheters that are placed directly into or near the cancer. Internal radiation therapy may allow for higher dose of radiation in a smaller area than might be possible with external radiation treatment. Internal radiation therapy includes high-dose-rate (HDR) brachytherapy (using a highly radiative source for a relatively short e.g. 10 to 20 minute amount of time over a number of intervals) and low-dose-rate brachytherapy (use of lower doses of radiation over a longer period).

Radiation therapy may also include systemic radiation therapies such as radioimmunotherapy and peptide receptor radionuclide therapy (PRRT).

Chemotherapy and radiation therapy may both be used either sequentially and/or simultaneously. Use of both therapies or one of chemotherapy or radiation therapy may be referred to as (chemo)radiation therapy. Use of both therapies may be referred to as chemoradiation therapy.

“Immunotherapy” as used herein relates to the treatment of cancer by modulation of the immune response of a subject. Said modulation may be inducing, enhancing, or suppressing said immune response. The term “cell based immunotherapy” relates to a breast cancer therapy comprising application of immune cells, e.g. T-cells, preferably tumour-specific NK cells, to a subject. In other examples, immunotherapy includes a checkpoint inhibitor, a bispecific T cell engager, a stimulator of interferon genes agonist, a RIG I like receptor agonist, a Toll-like receptor agonist, a cytokine, an antibody-cytokine fusion protein, or an antibody-drug conjugate.

As used herein, an “immune checkpoint inhibitor” means an agent that inhibits proteins or peptides (e.g. immune checkpoint proteins) which are blocking the immune system, e.g., from attacking cancer cells. In some examples, the immune checkpoint protein blocking the immune system prevents the production and/or activation of T cells. An immune checkpoint inhibitor can be an antibody or antigen-binding fragment thereof, a protein, a peptide, a small molecule, or combination thereof. Typically, the inhibitor interacts directly to a target immune checkpoint protein (or its ligand, where appropriate) and thereby disrupts its function/biological activity. For example, it may bind directly to a target immune checkpoint protein (or its ligand, where appropriate). In one example, direct binding to a target immune checkpoint protein (or its ligand, where appropriate) inhibits, prevents or reduces the formation of protein complexes which are needed for immune checkpoint protein function/biological activity.

A review describing immune checkpoint pathways and the blockade of such pathways with immune checkpoint inhibitor compounds is provided by Pardoll in Nature Reviews Cancer (April, 2012), pages 252-264. Immune checkpoint inhibitor compounds display anti-tumour activity by blocking one or more of the endogenous immune checkpoint pathways that downregulate an anti-tumour immune response. The inhibition or blockade of an immune checkpoint pathway typically involves inhibiting a checkpoint receptor and ligand interaction with an immune checkpoint inhibitor compound to reduce or eliminate the signal and resulting diminishment of the anti-tumour response.

The immune checkpoint inhibitor compound may inhibit the signalling interaction between an immune checkpoint receptor and the corresponding ligand of the immune checkpoint receptor. The immune checkpoint inhibitor compound can act by blocking activation of the immune checkpoint pathway by inhibition (antagonism) of an immune checkpoint receptor (some examples of receptors include CTLA-4, PD-1, and NKG2A) or by inhibition of a ligand of an immune checkpoint receptor (some examples of ligands include PD-L1 and PD-L2). In such examples, the effect of the immune checkpoint inhibitor compound is to reduce or eliminate down regulation of certain aspects of the immune system anti-tumour response in the tumour microenvironment.

In some examples, the immune checkpoint inhibitor inhibits the CTLA-4 pathway or the PD-L1/PD1 pathway. In some examples, the immune checkpoint inhibitor is an antibody. In some examples, the immune checkpoint inhibitor comprises an antibody that inhibits CTLA-4, PD1, or PD-L1. Immune checkpoint inhibitors, immune checkpoint inhibitors and examples thereof are provided in, e.g., WO 2016/062722.

In some examples, the immune checkpoint inhibitor is an anti-CTLA-4 antibody or derivative or antigen-binding fragment thereof. In examples, the anti-CTLA-4 antibody selectively binds a CTLA-4 protein or fragment thereof. Examples of anti-CTLA-4 antibodies and derivatives and fragments thereof are described in, e.g., U.S. Pat. Nos. 6,682,736; 7,109,003; 7,123,281; 7,411,057; 7,807,797; 7,824,679; 8,143,379; 8,491,895, and US 2007/0243184. In some examples, the anti-CTLA-4 antibody is tremelimumab or ipilimumab.

The immune checkpoint receptor cytotoxic T-lymphocyte associated antigen 4 (CTLA-4) is expressed on T-cells and is involved in signalling pathways that reduce the level of T-cell activation. It is believed that CTLA-4 can downregulate T-cell activation through competitive binding and sequestration of CD80 and CD86. In addition, CTLA-4 has been shown to be involved in enhancing the immunosuppressive activity of T_{Reg} cells.

In some examples, the immune checkpoint inhibitor is an anti-PD-L1 antibody or derivative or antigen-binding fragment thereof. In some examples, the anti-PD-L1 antibody or derivative or antigen-binding fragment thereof selectively binds a PD-L1 protein or fragment thereof. Examples of anti-PD-L1 antibodies and derivatives and fragments thereof are described in, e.g., WO 01/14556, WO 2007/005874, WO 2009/089149, WO 2011/066389, WO 2012/145493; U.S. Pat. Nos. 8,217,149, 8,779,108; US 2012/0039906, US 2013/0034559, US 2014/0044738, and US 2014/0356353. In some examples, the anti-PD-L1 antibody is MEDI4736 (durvalumab), MDPL3280A, 2.7A4, AMP-814, MDX-1105, atezolizumab (MPDL3280A), or BMS-936559.

The immune checkpoint receptor programmed death 1 (PD-1) is expressed by activated T-cells upon extended exposure to antigen. Engagement of PD-1 with its known binding ligands, PD-L1 and PD-L2, occurs primarily within the tumor microenvironment and results in downregulation of anti-tumor specific T-cell responses. Both PD-L1 and PD-L2 are known to be expressed on tumor cells. The expression of PD-L1 and PD-L2 on tumors has been correlated with decreased survival outcomes.

In some examples, the anti-PD-L1 antibody is MEDI4736, also known as durvalumab. MEDI4736 is an anti-PD-L1 antibody that is selective for a PD-L1 polypeptide and blocks the binding of PD-L1 to the PD-1 and CD80 receptors. MEDI4736 can relieve PD-L1-mediated suppression of human T-cell activation in vitro and can further inhibit tumor growth in a xenograft model via a T-cell dependent mechanism. MEDI4736 is further described in, e.g., U.S. Pat. No. 8,779,108. The fragment crystallizable (Fc) domain of MEDI4736 contains a triple mutation in the constant domain of the IgG1 heavy chain that reduces binding to the complement component C1q and the Fey receptors responsible for mediating antibody-dependent cell-mediated cytotoxicity (ADCC).

In some examples, the immune checkpoint inhibitor is an anti-PD-1 antibody or derivative or antigen-binding fragment thereof. In some examples, the anti-PD-1 antibody selectively binds a PD-1 protein or fragment thereof. In some examples, the anti-PD1 antibody is nivolumab, pembrolizumab, or pidilizumab.

NKG2A receptors are inhibitory receptors binding to HLA-E and expressed on tumor infiltrating cytotoxic NK and CD8 T lymphocytes. By expressing HLA-E, cancer cells can protect themselves from killing by NKG2A+ immune cells. HLA-E is frequently up-regulated on cancer cells of many solid tumors or hematological malignancies. Monalizumab (IPH2201), a humanized IgG4, blocks the binding of NKG2A to HLA-E allowing activation of NK and cytotoxic T cell responses. Examples of anti-NKG2A antibodies and derivatives and fragments thereof are described in WO 2016/041947, the content of which is hereby incorporated by reference in its entirety including, but not limited to, the sequence listings.

In some examples, the immune checkpoint inhibitor compound is a small organic molecule (molecular weight less than 1000 daltons), a peptide, a polypeptide, a protein, an antibody, an antibody fragment, or an antibody derivative. In some examples, the immune checkpoint inhibitor compound is an antibody. In some examples, the antibody is a monoclonal antibody, specifically a human or a humanized monoclonal antibody.

Monoclonal antibodies, antibody fragments, and antibody derivatives for blocking immune checkpoint pathways can be prepared by any of several methods known to those of ordinary skill in the art, including but not limited to, somatic cell hybridization techniques and hybridoma, methods. Hybridoma generation is described in Antibodies, A Laboratory Manual, Harlow and Lane, 1988, Cold Spring Harbor Publications, New York. Human monoclonal antibodies can be identified and isolated by screening phage display libraries of human immunoglobulin genes by methods described for example in U.S. Pat. Nos. 5,223,409, 5,403,484, 5,571,698, 6,582,915, and 6,593,081. Monoclonal antibodies can be prepared using the general methods described in U.S. Pat. No. 6,331,415 (Cabilly).

As an example, human monoclonal antibodies can be prepared using a XenoMouse™ (Abgenix, Freemont, CA) or hybridomas of B cells from a XenoMouse. A XenoMouse is a murine host having functional human immunoglobulin genes as described in U.S. Pat. No. 6,162,963 (Kucherlapati).

Methods for the preparation and use of immune checkpoint antibodies are described in the following illustrative publications. The preparation and therapeutic uses of anti-CTLA-4 antibodies are described in U.S. Pat. No. 7,229,628 (Allison), 7311910 (Linsley), and 8017144 (Korman). The preparation and therapeutic uses of anti-PD-1 antibodies are described in U.S. Pat. No. 8,008,449 (Korman) and U.S. Patent Application No. 2011/0271358 (Freeman). The preparation and therapeutic uses of anti-PD-L1 antibodies are described in U.S. Pat. No. 7,943,743 (Korman). The preparation and therapeutic uses of anti-TIM-3 antibodies are described in U.S. Pat. No. 8,101,176 (Kuchroo) and 8552156 (Tagayanagi). The preparation and therapeutic uses of anti-LAG-3 antibodies are described in U.S. Patent Application No. 2011/0150892 (Thudium) and International Publication Number WO2014/008218 (Lonberg). The preparation and therapeutic uses of anti-KIR antibodies are described in U.S. Pat. No. 8,119,775 (Moretta). The preparation of antibodies that block BTLA regulated inhibitory pathways (anti-BTLA antibodies) are described in U.S. Pat. No. 8,563,694 (Mataraza).

In some examples, the immune checkpoint inhibitor compound is a CTLA-4 inhibitor, a PD-1 inhibitor, a LAG-3 inhibitor, a TIM-3 inhibitor, a BTLA inhibitor, or a KIR inhibitor. In some examples, the immune checkpoint inhibitor compound is an inhibitor of PD-L1 or an inhibitor of PD-L2.

In some examples, the immune checkpoint inhibitor compound is an inhibitor of the PD-L1/PD-1 pathway or the PD-L2/PD-1 pathway. In some examples, the inhibitor of the PD-L1/PD-1 pathway is MEDI4736.

In some examples, the immune checkpoint inhibitor compound is an anti-CTLA-4 antibody, an anti-PD-1 antibody, an anti-LAG-3 antibody, an anti-TIM-3 antibody, an anti-BTLA antibody, an anti-KIR antibody, an anti-PD-L1 antibody, or an anti-PD-L2 antibody.

In some examples, the anti-CTLA-4 receptor antibody is ipilimumab or tremelimumab. In some examples the anti-PD-1 receptor antibody is lambrolizumab, pidilizumab, or nivolumab. In some examples, the anti-KIR receptor antibody is lirilumab.

Immune checkpoint inhibitors that may be administered to a subject include but are not limited to an anti-PD-1 antibody, anti-PD-L1 antibody, anti-LAG-3 antibody, anti-TIGIT antibody, anti-KLRB1 antibody, anti-LILRB2 antibody, anti-LILRB4 antibody, anti-LILRB2 and LILRB4 antibody and/or anti-TIM-3 antibody. Examples of immune checkpoint inhibitors include atezolizumab, ipimilumab, pembrolizumab, lambrolizumab (MK-3475, MERCK), nivolumab (BMS-936558, BRISTOL-MYERS SQUIBB), AMP-224 (MERCK), pidilizumab (CT-011, CURETECH LTD) and tislelizumab. Exemplary anti-PD-L1 antibodies include MDX-1105 (MEDAREX), MEDI4736 (MEDIMMUNE) MPDL3280A (GENENTECH) and BMS-936559 (BRISTOL-MYERS SQUIBB). Other examples include LILRB2 and LILRB4 antibodies described in US20190194327A1.

The inhibitor need not be an antibody, but can be a small molecule or other agent or compound. If the inhibitor is an antibody it may be a polyclonal, monoclonal, fragment, single chain, or other antibody variant construct. Inhibitors may target any immune checkpoint protein known in the art, including but not limited to, CTLA-4, PDL1, PDL2, PD1, B7-H3, B7-H4, BTLA, HVEM, TIM3, GAL9, LAG3, VISTA, KIR, 2B4, CD160, CGEN-15049, CHK1, CHK2, A2aR, and the B-7 family of ligands. Combinations of inhibitors for a single target immune checkpoint or different inhibitors for different immune checkpoints may be used. In particular the immune checkpoint therapy may be an inhibitor of one or more of CD274 (PD-L1), PDCD1LG2 (PD-L2), TIGIT, HAVCR2 (TIM-3), LAG-3, KLRB1, LILRB2 and/or LILRB4.

Breast cancer treatments include treatment by surgery, radiation therapy, or a combination of both, as well as systemic treatment by chemotherapy, endocrine therapy, checkpoint inhibitor therapy (or immunotherapy), or a combination thereof. Examples of drugs used for breast cancer chemotherapy include: Cytoxan® (Cyclophosphamide), Methotrexate, 5-Fluorouracil (5-FU), Adriamycin® (Doxorubicin), Prednisone, Nolvadex® (Tamoxifen), Taxol® (Paclitaxel), Leucovorin, Oncovin® (Vincristine), Thioplex® (Thiotepa), Arimidex® (Anastrozole), Taxotere® (Docetaxel), Navelbine®, (Vinorelbine tartrate), Gemzar® (Gemcitabine).

Examples of combination chemotherapy include the following: CMF (cyclophosphamide, methotrexate, and 5-fluorouracil); classic CMF (oral cyclophosphamide plus methotrexate and 5-fluorouracil); CAF or FAC (cyclophosphamide, Adriamycin® (doxorubicin), and 5-fluorouracil); AC (Adriamycin® and cyclophosphamide); ACT (Adriamycin® plus cyclophosphamide and tamoxifen); AC taxol (Adriamycin® plus cyclophosphamide and paclitaxel (Taxol®)); FACT (5-fluorouracil plus Adriamycin®, cyclophosphamide, and tamoxifen); A-CMF or Adria/CMF (4 cycles of Adriamycin® followed by 8 cycles of CMF); CMFP (CMF plus prednisone); CMFVP (CMF plus vincristine and prednisone); CAFMV (CAF plus methotrexate and vincristine); CMFVATN (CMF plus vincristine, Adriamycin®, thiotepa, and tamoxifen); MF (methotrexate plus 5-fluorouracil and leucovorin).

Medicines used to relieve side effects caused by chemotherapy include anti-nausea drugs (e.g., reglan), anti-anemia drugs (e.g., epoetin alfa [Procrit®, Epogen®]), and cell-protecting drugs (e.g., amifostin [Ethyol®]).

Examples of additional anticancer drugs that can be used in breast cancer therapy include: alkylating agents including cyclophosphamide (Cytoxan®), ifosphamide (Ifex®), melphalan (L-Pam®), thiotepa (Thioplex®), cisplatin (Cisplatinum®, Platinol®), carboplatin (Paraplatin®), and carmustine (BCNU; BiCNU®); antimetabolites including 5-Fluorouracil (5-FU) methotrexate and edatrexate; antitumor antibiotics including doxorubicin (Adriamycin®) and mitomycin C (Mutamycin®); cytotoxics including mitoxantrone (Novantrone®); vinca alkaloids including vincristine (Oncovin®), vinblastine (Velban®) and vinorelbine (Navelbine®); taxanes including paclitaxel (Taxol®) and docetaxel (Taxotere®); retinoids including fenretinide, corticosteroids including prednisone; antiestrogens including tamoxifen (Nolvadex®); male hormones including fluoxymesterone (Halotestin®); topoisomerase-I compounds including topotecan, irinotecan, 9-amino-camptothecin [9-AC]; anthrapyrazoles including biantrazole and losoxantrone; epidophylotoxins including etoposide and teniposide and angiogenesis inhibitors including compounds that block growth promoting receptors (e.g., PDGF-R and VEGF-R) such as sunitinib (Sutent®).

Hormonal medications also may be used in treatment. If the patient is ER/PR-negative, then chemotherapy usually is given without hormone therapy, however, hormone therapy may be suitable for patients who are in poor health or who have a short projected survival time. In addition to tamoxifen (Nolvadex®), such drugs include: aromatase inhibitors including anastrozole (Arimidex®) and aminoglutethimide (Cytadren®); luteinizing hormone-releasing hormone-inhibiting compounds including goserelin (Zoladex®) and leuprolide (Lupron®); progestins including megestrol acetate (Megace®) and medroxyprogesterone acetate (Provera®); and androgens including fluoxymesterone (Halotestin®), testolactone (Teslac®), and testosterone enanthate (Delatestryl®).

For tumours that are c-erbB2 (HER2) positive, trastuzumab (Herceptin®), a humanized monoclonal antibody against the extracellular domain of HER2, can be used.

When the disease is prostate cancer the treatment may include one or more of surgery (e.g., radical proctectomy, pelvic lymphadenectomy, radical prostatectomy, transurethral resection of the prostate (TURP), excision, dissection, and tumour biopsy/removal), radiation therapy, hormone therapy (e.g., using GnRH antagonists, GnRH agonists, antiandrogens such as Goserelin (Zoladex®), Leuprorelin acetate (Prostap® or Lutrate®), Triptorelin (Decapeptyl® or Gonapeptyl Depot®), Buserelin acetate (Suprefact®), Histrelin (Vantas®), Degarelix (Firmagon®), Bicalutamide (Casodex®), Cyproterone acetate (Cyprostat®), Flutamide (Drogenil®), Abiraterone acetate (Zytiga®), or Nilutamide (Nilandron®)), ultrasound, chemotherapy (e.g. Docetaxel (Taxotere®), Cabazitaxel (Jevtana®), Strontium-89 (Metastron®), Samarium-153 (Quadramet®), Enzalutamide (Xtandi®), Radium-223 dichloride (Xofigo®), or Apalutamide (Erleada®)), Steroids (e.g. Prednisolone, Dexamethasone, Hydrocortisone); Sipuleucel-T (Provenge®) (to treat advanced, recurrent prostate cancer), or Ketoconazole, optionally in combination with a treatment selected from the group consisting of: radical prostatectomy, external beam radiotherapy/Brachytherapy (with or without hormone therapy), High Intensity Focused Ultrasound (HIFU), Cryotherapy and Trans-urethral resection of the prostate (TURP), Monoclonal antibody therapies (e.g. Pembrolizumab (keytruda), Avastin (bevacizumab), Erbitux (cetuximab), Rituxan (rituximab) and Herceptin (trastuzumab)), or combinations thereof.

When the disease is endometrial cancer the treatment may include one or more of treatments described in WO2016071520A1. For example, hysterectomy, bilateral salpingo-oophorectomy, radical hysterectomy, mTOR inhibitor therapy and/or Lenvatinib therapy.

### Databases and Models

In some examples, the method provided herein may be used to generate a database or classification model for classifying genetic aberrations associated with a disease to stratify a subject suffering from a disease. For example, by determining a score for each DEG that overlaps for a representative Group A and Group B genomic aberration and/or one or more further Group A and Group B genomic aberrations (shared set of DEGs) for a plurality of control subjects suffering from the disease a database of scores can be generated for a genomic aberration and/or disease. In addition, at each tier from tiers 1 to 6, a signature of one or more group-specific biomarkers can be determined and entered into a database or model and a subject sample can be analysed and compared to the model or database to stratify the patient into a group or subgroup of each tier based on levels of the biomarkers in the subject sample.

By taking a subject sample from a subject suffering from the same disease and determining the expression level of specific genes selected from the shared set of DEGs (disease-specific gene signature), the expression levels for the specific genes can then be compared to the score assigned to each gene in the database to provide a risk score for the subject. Alternatively or in addition to, the levels of one or more group-specific biomarkers can be compared to a model or database including the gene signatures for each group or subgroup of each tier determined as described herein for a control set of subjects.

Using statistical analysis of the risk score calculated for each of the plurality of control subjects for the same genes as the disease-specific gene signature it is possible to determine a risk score that may be considered high or low for the disease-specific gene signature. For example, a high and low risk score for a disease-specific gene signature may be determined by application of receiver-operating-characteristic (ROC) curve analysis to the scores calculated for the plurality of control subjects.

For example, there is provided a method for producing a database for stratification of a subject suffering from a disease, the disease comprising one or more genomic aberrations, the method comprising:
a. identifying genes in a plurality of control subjects suffering from the disease that undergo a change of expression in response to a first genomic aberration and selecting the genes that undergo a change of expression in response to the first genomic aberration to provide a first set of differentially expressed genes (DEGs) associated with the first genomic aberration;
b. identifying DEGs of the first set of DEGs that overlap with DEGs of a control set of DEGs for a control genomic aberration and selecting the overlapping DEGs to form a first set of overlapping DEGs;
c. comparing the fold direction of change of expression of each DEG of the first set of overlapping DEGs to a fold direction change of expression of the corresponding DEG of the control set of DEGs;
d. classifying the first genomic aberration into a first or second group wherein:
   i. the first group comprises at least 51% overlapping DEGs that comprises a fold direction of change of expression that is the same as the fold direction of change of expression as the corresponding DEG of the control genomic aberration (Group A);
   ii. the second group comprises at least 51% overlapping DEGs that comprise a fold direction of change of expression that is inverse to the fold direction of change of expression of the corresponding DEG of the control genomic aberration (Group B);
e. classifying at least one genomic aberration associated with the disease as Group A and at least one second genomic aberration associated with the disease as Group B;
f. selecting a representative Group A genomic aberration and a representative Group B genomic aberration;
wherein each representative genomic aberration is selected based on the frequency of occurrence of the genomic aberration in the disease and/or the number of DEGs associated with the genomic aberration.
g. determining DEGs associated with the representative Group A genomic aberration and/or one or more further Group A genomic aberration that overlap with DEGs associated with the representative Group B genomic aberration and/or one or more further Group B genomic aberration and selecting the overlapping DEGs to form a second overlapping set of DEGs for each of the plurality of control subjects;
h. optionally performing statistical approaches such as Lasso, univariate, and/or multivariate Cox regression analyses to identify prognosis-related DEGs from the second overlapping set of DEGs for a disease-specific gene signature.
i. determining an expression level for each DEG of each second overlapping set of DEGs based on a level of RNA transcript for each DEG for each of the plurality of control subjects;
j. calculating a score for each DEG of the second overlapping set of DEGs based on the fold direction change of expression and/or the expression level of each DEG for each of the plurality of control subjects.

Calculating the score may be done using any one of the methods as described herein. For example, calculating the score may include:
a. sorting the expression level of each DEG of the second overlapping set of DEGs of the plurality of control subjects that are upregulated for the representative Group A genomic aberration, one or more further group A genomic aberrations and/or control genomic aberration in ascending order of the expression level; and
   dividing the sorted expression levels into 1 to n fractions based on a dynamic range of the expression levels for each of the plurality of control subjects; and
   assigning a relative expression value for each fraction, wherein the relative expression value is 1 to n for each corresponding fraction from lowest to highest expression level;
b. sorting the expression level of each DEG of the second overlapping set of DEGs of the plurality of control subjects are downregulated for the representative Group A genomic one or more further group A genomic aberrations and/or control genomic aberration in ascending order of the expression level; and
   dividing the sorted expression levels into 1 to n fractions based on a dynamic range of the expression levels for each of the plurality of control subjects; and
   assigning a relative expression value for each fraction, wherein the relative expression value is 1 to n for each corresponding fraction from highest to lowest expression level;
wherein the relative expression value is the score.

A risk score may then be calculated by selecting two or more DEGs of the second overlapping set of DEGs to form a disease-specific gene signature and calculating the sum of the relative expression values assigned to each DEG of the disease-specific gene signature to provide the risk score for each of the plurality of control subjects. The disease-specific gene signature comprises at least one DEG that has a direction of fold change of expression that is inverse between the representative Group A and representative Group B genomic aberrations.

The calculation of the risk score may be done by:
calculating the difference between:
   the sum of the expression level of each DEG of the disease-specific genes of each of the plurality of control subjects that are upregulated for the corresponding DEG of the representative Group A genomic aberration, one or more further group A genomic aberrations and/or control genomic aberration genomic aberration; and
   the sum of the expression level of each DEG of the disease-specific genes for each of the plurality of control subjects that are downregulated for the corresponding DEG of the representative Group A genomic aberration, one or more further group A genomic aberrations and/or control genomic aberration.

In some examples, the score may be calculated first by:
calculating weights from the effect of each gene included in the disease-specific gene signature on the clinical outcome (based on the coefficient value of each gene) through the following formula:

$$Score = {\sum }_{i = 1}^{n} Exp i * β i ,$$

where Expi is the expression value of each gene, and βi is the regression coefficient of the multivariate Cox analysis for each gene that makes up the disease-specific gene signature.

In some examples, calculating a risk score may include:
calculating a ratio of expression levels for each DEG of the disease-specific genes for each of the plurality of control subjects that are upregulated for the corresponding DEG of the representative Group A genomic aberration, one or more further group A genomic aberrations and/or control genomic aberration to;
the expression levels for each DEG of the disease-specific genes for each of the plurality of control subjects that are upregulated for the corresponding DEG of the representative Group A genomic aberration, one or more further group A genomic aberrations and/or control genomic aberration.

The method may then further comprise determining a relatively high or low risk score by statistical analysis of the risk score for each of the plurality of control subjects for each disease specific gene signature.

Given the above, also provided herein is a database that comprises a plurality of scores for DEGs associated with a disease comprising one more genomic aberrations.

There is also provided a method stratifying a subject suffering from the disease comprising one or more genomic aberrations, the method comprising:
selecting two or more DEGs comprised within the database as described herein to form a disease-specific signature, wherein the disease-specific gene signature comprises at least one DEG that has a direction of fold change of expression that is inverse between the representative Group A and representative Group B genomic aberrations;
providing an expression level of each gene of the disease-specific gene signature for the subject
comparing the expression level of each DEG of the disease-specific gene signature of the subject to the expression levels of each of the corresponding DEGs of the database;
d. assigning a relative expression value to each DEG of the disease-specific gene signature of the subject based on the expression value assigned to the corresponding DEG in the database; and
e. calculating the sum of the relative expression values assigned to each DEG of the disease-specific DEG set of the subject to provide a risk score for the subject.

In some examples, the subject and plurality of control subjects used to form the database suffer from the same disease comprising at least one of the same genomic aberrations as at least one of the plurality of control subjects.

The subject may then be stratified into a high or low risk group based on the risk score. As mentioned above, a high or low risk score may be determined by calculating a risk score for each of the plurality of control subjects for the same DEGs as the disease-specific gene signature and using statistical analysis to define a high or low score.

As described above, a number of distinct subgroups and/or subsets may be defined using the methods described herein. The specific scores for each gene and for each specific subgroup and/or subset (i.e. for each group defined for tier 1, tier 2, tier 3, tier 4, tier 5 and/or tier 6) may be stored in the database and applied by comparison to a subjects data.

In addition, an immune risk score database for immune associated genes and/or metastatic risk score database for metastasis associated genes for a disease may also be created and used to stratify subjects.

Given the above, there is provided herein a database comprising scores for each gene identified for genomic aberrations associated with a disease for a plurality of subjects suffering from a disease comprising one or more genomic aberration as described herein that have been classified as described herein.

Furthermore, there is provided a database comprising immune scores for immune associated genes and/or metastatic risk score database for metastasis associated genes of a plurality of subjects suffering from a disease comprising one or more genomic aberration as described herein calculated as described herein.

### Computer Implemented Methods

It will be apparent to a person skilled in the art that the methods described herein or parts thereof can be carried out using a computer. As such the methods provided herein may be computer implemented methods.

Therefore there is also provided, a computer-readable storage medium comprising instructions which, when executed by a computer, cause the computer to carry out one or more of the steps of any of the methods described herein.

In addition, there is provided a computer program comprising instructions which, when the program is executed by a computer, cause the computer to carry out the method of stratifying a patient as described herein.

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein are computer readable storage media with an executable program stored thereon, where the program instructs a microprocessor to perform a method described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

Also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some examples, methods described herein (e.g., identifying, comparing, classifying, normalizing, selecting, calculating and/or determining associated genes and immune associated genes) are performed by automated methods. In some examples, one or more steps of a method described herein are carried out by a microprocessor and/or computer, and/or carried out in conjunction with memory. In some examples, an automated method is embodied in software, modules, microprocessors, peripherals and/or a machine comprising the like, that perform methods described herein. As used herein, software refers to computer readable program instructions that, when executed by a microprocessor, perform computer operations, as described herein.

DEGs, immune associated genes, metastasis associated genes, mutational status and expression data may be generally referred to as “data” or “data sets.”

Machines, software and interfaces may be used to conduct methods described herein. Using machines, software and interfaces, a user may enter, request, query or determine options for using particular information, programs, which can involve implementing statistical analysis process, statistical significance process, statistical process, iterative steps, validation process, and graphical representations, for example. In some examples, a data set may be entered by a user as input information, a user may download one or more data sets by suitable hardware media (e.g., flash drive), and/or a user may send a data set from one system to another for subsequent processing and/or providing an outcome.

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A user may, for example, place a query to software which then may acquire a data set via internet access, and in certain examples, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical process, one or more statistical analysis process, one or more statistical significance process, iterative steps, one or more validation process, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

A system may include software useful for performing a method or part of a method described herein, and software can include one or more modules for performing such methods. The term “software” refers to computer readable program instructions that, when executed by a computer, perform computer operations. Instructions executable by the one or more microprocessors sometimes are provided as executable code, that when executed, can cause one or more microprocessors to implement a method described herein. A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue.

A system may include one or more microprocessors. A microprocessor can be connected to a communication bus. A computer system may include a main memory, often random access memory (RAM), and can also include a secondary memory. Memory in some examples comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

### Companion Diagnostic

In some examples, the methods described herein may be used as a companion diagnostic. “Companion diagnostic” refers to a medical device or method which provides information that is essential for the safe and effective use of a corresponding drug or biological product. The method helps a health care professional determine whether a particular therapeutic product's benefits to patients will outweigh any potential serious side effects or risks.

In some examples, the clinical performance of the companion diagnostic is the ability of the method to distinguish treatment responders from non-responders. Companion diagnostics can: (i) identify patients who are most likely to benefit from a particular therapeutic product; (ii) identify patients likely to be at increased risk for serious side effects as a result of treatment with a particular therapeutic product; and/or (iii) monitor response to treatment with a particular therapeutic product for the purpose of adjusting treatment to achieve improved safety or effectiveness. The clinical performance of the companion diagnostic not only directly affects the number of patients who are potentially eligible for treatment but also affects the net benefit enrichment achieved, as patients who are selected by the companion diagnostic and are non-responders also receive treatment, thereby reducing the observed average response.

As such, there is provided herein apparatus for carrying out one or more of the methods described herein for use as a companion diagnostic. In some examples, the apparatus includes a computer-readable storage medium comprising instructions which, when executed by a computer, cause the computer to carry out one or more of the steps of any of the methods described herein.

For example, an apparatus may be provided that can group a subject into one or more of the tier groups described herein prior to administering a treatment. Based on the outcome of the method and stratification of the subject, suitability of a specific treatment for that subject can be determined.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Example 1

### Materials and Methods

Publicly available cancer datasets with patient's tumour molecular profiling data (including genomic data, gene expression data, and protein expression data), clinical outcome data, medical record data, patient-reported data and pathology report data were used to design and validate the classification methods as described herein.

The example described here mainly uses two large-scale human breast cancer multiomics datasets—The Molecular Taxonomy of Breast Cancer International Consortium (METABRIC) and The Cancer Genome Atlas (TCGA) breast cancer datasets. The TCGA breast cancer Microarray and RNA-Seq gene expression profiles, gene somatic mutations, somatic copy number alterations (SCNAs), protein expression profiles, and clinical data were downloaded from the genomic data commons data portal (https://portal.gdc.cancer.gov/), and the METABRIC gene expression profiles, gene somatic mutations, SCNAs, and clinical data from cBioPortal (http://www.cbioportal.org).

TCGA breast cancer dataset has data from 1101 human breast cancer patients. In the TCGA breast dataset an Illumina HiSeq 2000 RNA Sequencing platform (Illumina, Inc., San Diego, CA, USA) was used to generate mRNA-seq data. The mRNA expression profile data were initially identified in the TCGA database according to the HUGO (Human Genome Organisation) Gene Nomenclature Committee (HGNC, http://www.genenames.org/), which includes 19,034 protein-coding gene annotations. In the TCGA breast dataset, the protein expression profiles were obtained using the Reverse Phase Protein Array (RPPA) technology for functional proteomics studies. Biospecimens were collected from newly diagnosed patients with invasive breast adenocarcinoma undergoing surgical resection and had received no prior treatment for their disease (chemotherapy or radiotherapy).

The METABRIC dataset comprises a collection of over 2,000 clinically annotated primary fresh frozen human breast cancer specimens and a subset of normals that passed initial selection criteria from tumour banks in the UK and Canada. The tumours in the original METABRIC cohort were collected between 1977-2005 from five centres in the UK and Canada. The original annotation of these tumours was based on the primary pathology reports, with obvious differences in terminology for the classification of histological tumour types over time and between the five contributing centres. In the METABRIC dataset, 177 genes were sequenced in 2,000 primary breast tumours with copy number aberration (CNA), gene expression and long-term clinical follow-up data. Sample processing, DNA extractions and quality assessment were based on the protocols described in the METABRIC publication³. DNA and RNA were isolated from samples and hybridized to the Affymetrix SNP 6.0 and Illumina HT-12 v3 platforms for genomic and transcriptional profiling, respectively. Somatic mutation data in TCGA were generated by whole exome sequencing while in METABRIC they were generated by targeted exome sequencing. Immunohistochemistry-based (IHC) scoring of ER status was, where available, used to classify ER-positive (ER+) and ER-negative (ER−) tumours. In the METABRIC breast cancer dataset maximum clinical follow-up time is 351 months. In the METABRIC breast cancer dataset nearly all oestrogen receptor (ER)-positive and/or lymph node (LN)-negative patients did not receive chemotherapy, whereas ER-negative and LN-positive patients did. Additionally, none of the HER2⁺ patients received trastuzumab. As such, the treatments were homogeneous with respect to clinically relevant groupings. In the METABRIC breast cancer dataset, while not individually curated, hormone therapy patients were treated with tamoxifen and/or aromatase inhibitors, while CT patients were most commonly treated with cyclophosphamide-methotrexate-fluorouracil (CMF), epirubicin-CMF, or doxorubicin-cyclophosphamide.

The effectiveness of the prognostic prediction system was validated with GEO databases—GSE7390, GSE1456, GSE20685, and GSE9195.

The coding for the analysis is provided below:

```
MUT_data<−read.table( )
exprs_data<−read.table( )
#only take the columns that have genomic abberation and expression data
exprs_new<−c( )
for (i in 1:dim(MUT_data)[2]){
  exprs_new<−cbind(exprs_new,exprs_data[,colnames(exprs_data)==colnames(MUT_data)[i]])
}
colnames(exprs_new)<−colnames(MUT_data)
rownames(exprs_new)<−rownames(exprs_data)
norm<−log2(exprs_new)
#Make a table with row names of genes and expression values
Norm_exprs<−cbind(rownames(norm),norm)
colnames(Norm_exprs)[1]<−c(“ID”)
Norm_exprs_IDs<−as.data.frame(Norm_exprs)
#Run a loop to compute the stats for all the possible combinations of differentially expressed genes
TP53<−data.frame(na.omit(MUT_data[“GROUP”,]))
library (gtools)
transpose_Exp = t(Norm_exprs_IDs)
Exp_temp = merge(transpose_Exp, t(TP53), by = 0)
wilcox.results = data.frame(logFC = rep(0, nrow(Norm_exprs_IDs)),
    t = rep(0, nrow(Norm_exprs_IDs)),
    Mean.WT = rep(0, nrow(Norm_exprs_IDs)),
    Mean.Mut = rep(0, nrow(Norm_exprs_IDs)),
    P.value = rep(0, nrow(Norm_exprs_IDs)),
    Adj.P.Val = rep(0, nrow(Norm_exprs_IDs)))
rownames(wilcox.results) = rownames(Norm_exprs_IDs)
temp = c( )
for (i in 2:ncol(Exp_temp)){
  temp = t.test(as.numeric(Exp_temp[,i]) ~ Exp_temp[,])
  wilcox.results[i-1,5] = temp$p.value
  wilcox.results[i-1,2] = temp$statistic
  wilcox.results[i-1,1] = foldchange2logratio(foldchange(temp$estimate[[1]], temp$estimate[[2]]), base = 2)
  wilcox.results[i-1,3] = temp$estimate[[1]]
  wilcox.results[i-1,4] = temp$estimate[[2]]
}
wilcox.results[,6] = p.adjust(wilcox.results[,5], method = “fdr”)
```

### Results

This example shows how this first-of-its-kind multi-tier classification approach helps refine the biology and prognosis at each tier by separating breast tumours into different subgroups. This proactive, holistic multi-tier classification method enabled dissecting each breast tumour's biology and prognosis in detail.

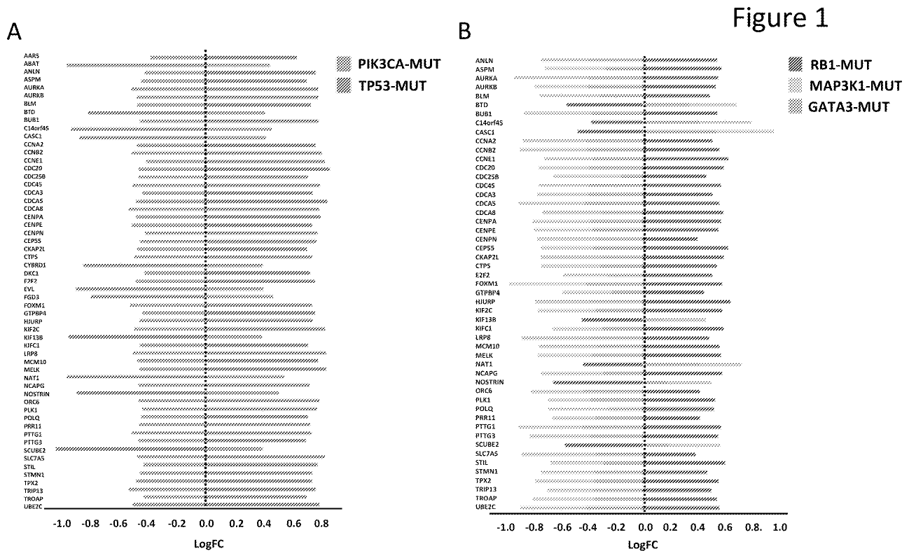

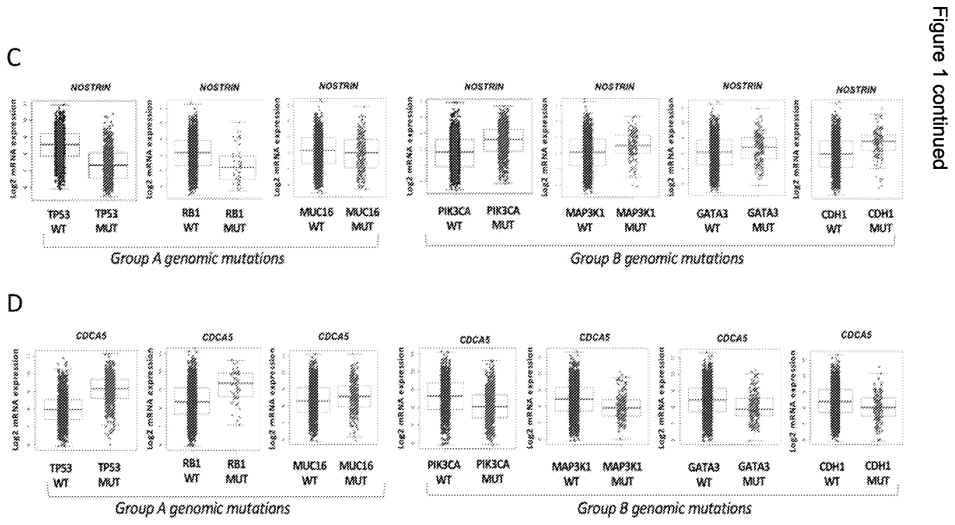

As mentioned earlier, the methods described herein are founded on a highly significant scientific discovery made by the inventor. The FIGS. 1A-1D shows there are a group of genomic aberrations in breast cancer following inverse downstream transcriptional effects [the fold-change direction of significant differentially expressed genes (DEGs)—DEGs were considered significant using a threshold of FDR<0.05] to the TP53 gene mutations induced transcriptional changes. TP53 is the most frequently mutated gene in human cancer, with greater than half of all tumours exhibiting mutation at this locus. TP53 mutations are identified in approximately 30%-40% of primary breast cancer patients, with a high prevalence of around 60% in triple-negative breast cancers (TNBC). Previous studies have suggested that TP53 status is crucial for the response of cancer patients to multiple anticancer therapies. In addition, TP53 mutations may be causally linked to drug resistance and failed treatment and are closely related to poor prognosis, making it an attractive therapeutic target and marker to predict therapy sensitivity in breast cancer.

The methods described herein identify the breast cancer-specific TP53 gene mutations-induced characteristic downstream transcriptional changes [the fold-change direction of significant DEGs with FDR adjusted p-value <0.05] and compares the direction of transcriptional change with those induced by other frequent genomic aberrations in breast cancer. The methods classify any genomic aberrations that broadly follow similar transcriptome expression patterns (the fold-change direction of shared/overlapping significant DEGs) to TP53 gene mutations as Group A genomic aberrations. The genomic alterations that broadly follow inverse transcriptome expression patterns (the fold-change direction of shared/overlapping DEGs) to TP53 gene mutations (Group A genomic aberrations, in general) are classified as Group B genomic aberrations.

Gains and losses of DNA are highly prevalent and widespread in cancer, leading to chromosomal instability and aneuploidy. The copy number alterations (CNAs) contribute to cancer initiation, progression and therapeutic resistance. It is noteworthy that CNAs often involve oncogenes and tumour suppressors (i.e., driver genes), which can directly affect cancer development and disease progression. CNAs are highly heterogenous and significantly contribute to genomic complexity associated with cancers.

Notably, most somatic copy number alterations (SCNAs) follow similar downstream transcriptome expression patterns (i.e. the similar fold-change direction of shared/overlapping significant DEGs) to TP53 gene mutations.

Also, as shown in FIGS. 1A, 1C and 1D, the transcriptional changes associated with PIK3CA, the gene most widely (~40% of patients with HR-positive, HER2-negative breast cancer, have activating mutations in the gene PIK3CA) affected in breast cancers, follow an inverse relationship to TP53 gene mutation-associated transcription pattern. Thus, grouped as Group B genomic alterations. Other gene mutations grouped as Group B genomic alterations in breast cancer include GATA3, CDH1, MAP3K and AKT gene mutations (a complete list of Group A and Group B genomic aberrations for breast cancer is provided in Table 1). TP53 gene mutations were selected as Group A representative genomic aberration, whereas PIK3CA gene mutations were selected as Group B representative genomic aberration being among the most frequently mutated gene (with the highest mutation frequency) with the highest number of statistically significant (up to FDR adjusted p-value <0.05) DEGs amongst other Group B genomic aberrations.

Importantly, these findings were found to be highly consistent and robust. A similar inverse relationship in gene expression changes was seen with several breast cancer datasets between the Group A and Group B genomic aberrations (FIGS. 1A-1D).

The shared (overlapping) set of DEGs (RNA transcripts) between the representative genomic aberrations from Group A (TP53 gene mutation) and Group B (PIK3CA gene mutations) were determined based on the METABRIC breast cancer dataset and the TCGA breast cancer dataset, which involved selecting the statistically significant (with FDR adjusted p-value <0.05) DEGs between each group's representative genomic aberration' DEGs list [Table 4 showing shared set of DEGs from top 200 statistically significant (with FDR adjusted p-value <0.05) DEGs from breast cancer representative Group A and Group B genomic aberration's (i.e., TP53 and PIK3CA gene mutations, respectively) DEGs list using the METABRIC breast cancer dataset].

Almost 90% of the shared (overlapping) set of DEGs between the TP53 and PIK3CA gene mutations were found to follow an inverse relationship regarding their fold-change expression direction (Table 4). That means most (>90%) of the DEGs that are upregulated in TP53 mutant tumours are downregulated in PIK3CA-mutant tumours and vice versa. A Gene Set Enrichment Analysis (GSEA) of the gene sets exclusively upregulated with TP53 gene mutations (and thus downregulated with PIK3CA gene mutations) from the shared (overlapping) set of DEGs revealed that the majority of the significantly upregulated gene sets (and downregulated with PIK3CA gene mutations) were related to critical tumour growth-supporting pathways. These included cell cycle, DNA replication, RNA transport, DNA repair, spliceosome and ribosome-biogenesis-associated molecular pathways.

It is to be noted that most CNAs (which are a significant contributor to genomic complexity associated with cancers) and other frequent cancer gene mutations belonging to Group A genomic aberration follow TP53 mutation-associated transcriptional pattern (i.e. the similar fold-change direction of shared/overlapping statistically significant DEGs), which as discussed above is associated with the upregulation of critical tumour growth-supporting pathways. Notably, PIK3CA gene mutations that are recently suggested to be associated with a better clinical outcome in some patients showed inverse transcription patterns to TP53 gene mutations (fold-change direction of shared/overlapping statistically significant DEGs) and downregulation of central tumour growth-supporting pathways. This indicated a clinical significance of the shared set of DEGs between the representative Group A (TP53 gene mutation) and Group B (PIK3CA gene mutations) genomic aberrations, as these gene sets represent a shared central factor associated with upregulation of critical tumour growth-supporting pathways and poor clinical outcomes in breast cancer. Moreover, this can also help in better understanding breast cancer-associated genomic complexity. Accordingly, a molecular gene-expression signature including two or more combined groups of RNA transcripts/genes was derived from the shared (overlapping) DEG sets and a risk score representing the breast cancer genomic complexity was derived. When calculating the risk score (referred to as AG-Score hereafter), the weight of the RNA transcripts (belonging to the derived molecular gene signature) was either +1 or −1, depending on their association with TP53 mutation status. Further statistical approaches (univariate and multivariate Cox regression analyses) were performed to identify the prognosis/survival-related DEGs for a breast-specific gene signature (Table 7).

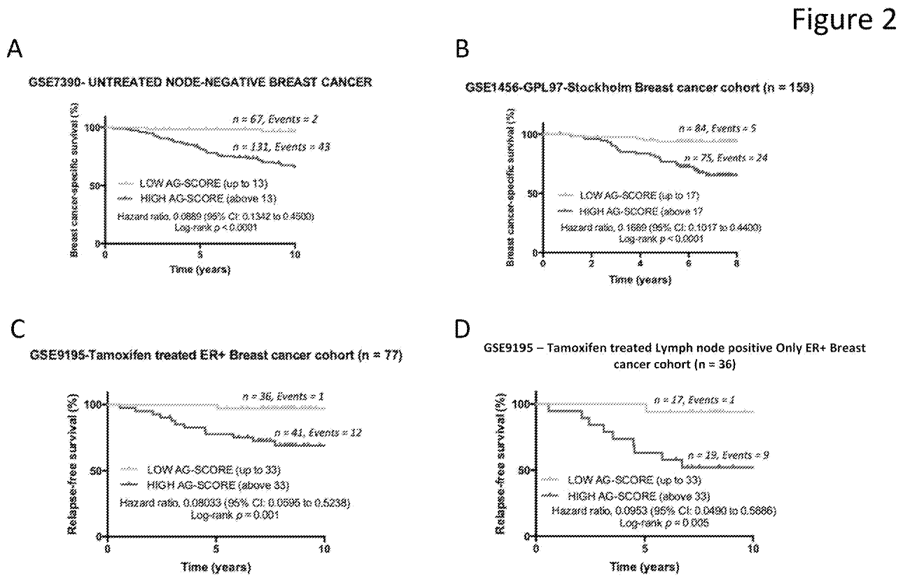

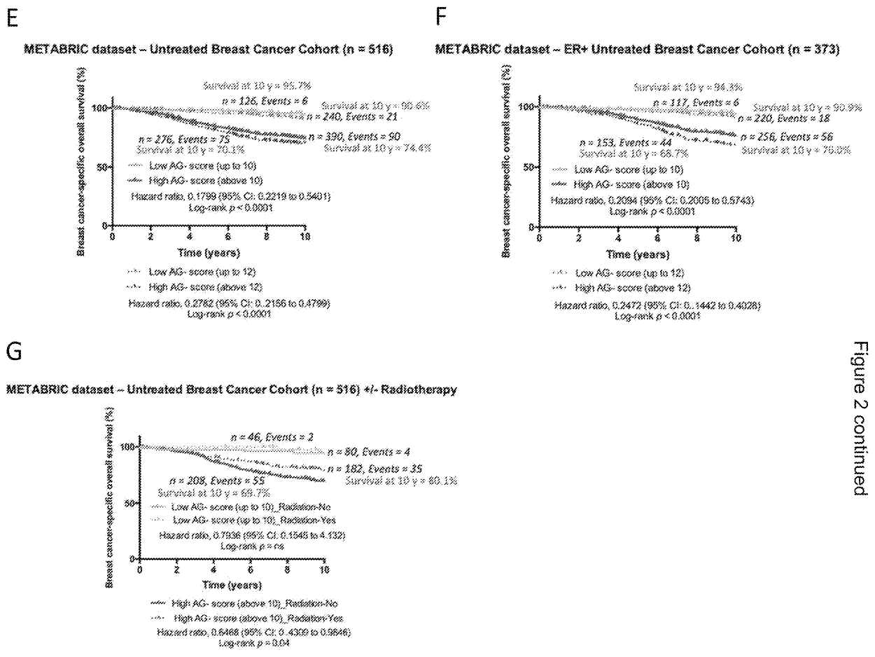

Tier-1 classification—The method involved tentatively grouping a breast tumour sample into a low or high-risk group based on the AG-score (risk score). Some examples of Tier-1 classification are provided in FIGS. 2A-2G using several breast cancer datasets with available clinical outcomes and tumour molecular profiling data. AG scores derived from different gene signatures (for example, using three, four, five, six or eight combined groups of RNA transcripts/genes from the shared set of DEGs between the representative Group A and Group B genomic aberrations were used to stratify breast cancer patients as low and high-risk, respectively, where low AG-scores were predictive of significantly improved clinical indications, such as overall survival, disease-free survival or relapse-free survival, etc. It was also seen that (FIGS. 2A-2G), an AG-score may be predictive (negatively correlated) with the subject's clinical outcome, for example, overall survival in breast cancer.

While the annual mortality risk for ER-negative breast cancer decreases following the first five years after diagnosis, the annual rate remains constant for ER+ patients. Women with ER+ early-stage disease treated with five years of adjuvant endocrine therapy have a persistent risk of recurrence and death from breast cancer for at least 20 years after diagnosis. Thus, (FIGS. 2A-2G), low-risk patients (Low AG-score group) were defined as those with >90% survival chances (such as overall survival, disease-free survival or relapse-free survival) for up to 8 or 10 years after a breast cancer diagnosis. This is because the risk of recurrence and death from breast cancer is usually significantly higher in all ER+ patients beyond ten years after a breast cancer diagnosis, even for those treated with adjuvant therapies, making it difficult to assess risk beyond 10 years (at least at this classification tier).

(FIGS. 2A-2G), The patients stratified using AG-scores were those who have not received any systemic adjuvant therapy (hormone therapy or chemotherapy) or radiotherapy, as well as those who have been given some therapy (hormone therapy or chemotherapy or radiotherapy). The method can thus be used to find which group of patients might benefit from specific treatments (for example, FIG. 2G shows that the high-risk breast cancer patient subset significantly benefits from radiotherapy). Various cut-off values for AG scores can classify a subject as low or high-risk (FIGS. 2E-2F).

As illustrated in the example (FIGS. 2A-2G), the breast cancer patients stratified using AG-scores into low and high-risk groups can be those which are hormone receptor-positive (ER+), hormone receptor negative (ER−), HER2+ or TNBC (i.e. ER- and HER2−). The ER, PR, and HER2 statuses are determined concurrently with this tier-based molecular classification of a breast tumour sample. ER, PR and HER2 status in some breast cancer datasets are determined at the nucleic acid level (e.g., by microarray to determine the oestrogen receptor (ESR1), progesterone receptor (PGR), or HER2 gene expressions. For some breast cancer datasets, ER, PR and HER2 status are determined at the protein level (e.g., by immunochemistry, as described in, for example, the METABRIC breast cancer dataset).

As illustrated in the example (FIGS. 2A-2G), the breast cancer patients stratified using AG-scores into low and high-risk groups can be from various lymph nodal status types, i.e., breast cancer patients could be lymph node-negative (with no lymph node involvement) or lymph node-positive (with either 1-3, >1, or >3 lymph node involvements).

Statistical approaches (GSEA, over-representation analysis and/or pathway/network enrichment analyses, etc.) were used to gain insights into the low and high-risk groups show group-specific biology's. For example, the GSEA revealed the underlying biological/molecular pathways enriched in the high-risk group (as compared to the low-risk group) patients, including cell cycle, DNA replication, DNA repair, Biosynthesis of amino acids, mTOR signalling, carbon metabolism pathways, etc. Whereas the low-risk group showed enrichment in molecular pathways, such as focal adhesion, ECM-receptor interaction, Circadian rhythm, Valine, leucine and Isoleucine degradation, MAPK signalling pathways, etc. These results are highly robust and reproducible as GSEA low, and high-risk group analysis across different breast cancer datasets revealed consistent pathways being enriched across the low and high-risk groups.

Gene expression analysis has a few weak points—it cannot evaluate the genes' active status. In contrast, reverse phase protein array (RPPA) analysis allows the investigation of potential targets and biomarkers at the protein level (which could also lead to the development of immunohistochemical assays). A further strength of RPPA is that it reflects protein function; proteins are the most actionable and druggable cellular components. Thus, it was sought to determine whether functional proteomics could define molecular biomarkers across the low and high-risk groups identified above.

Consistent with the GSEA, the high-risk group showed upregulation of mTOR signalling and cell cycle pathways at the RPPA levels, along with altering other pathways. Notably, the protein and phosphorylation (associated with functional proteomics) levels of several key enzymes related to these molecular pathways were significantly altered in the high-risk groups. For example, key signalling proteins related to mTOR pathways, such as S6, phospho-4EBP1, eIG4G, ASNS, etc., were significantly higher in high-risk groups. Similarly, key signalling proteins related to cell cycle and proliferation pathways, such as levels of enzymes cyclin B1 and FoxM1, were significantly higher in high-risk groups consistent with the GSEA described above.

Tier-2 classification—Given the anticipated vast clinical significance (as discussed above) of the shared set of DEGs between the representative Group A (TP53 gene mutation) and Group B (PIK3CA gene mutations) genomic aberrations that were used for AG-score calculations (representing the tumour's genomic complexity) and for stratifying breast patients into low and high-risk groups—it was next sought to find out if the methods could be further exploited to find the nuance, context and significance of various genomic aberrations. Thus, the high and low-risk groups were further sub-grouped based on the breast cancer-specific tumour's genomic profile. For example, the high and low-risk groups identified above were subclassified based on the representative genomic aberrations from Group A (TP53 gene mutations) and/or Group B (PIK3CA gene mutations) genomic aberrations.

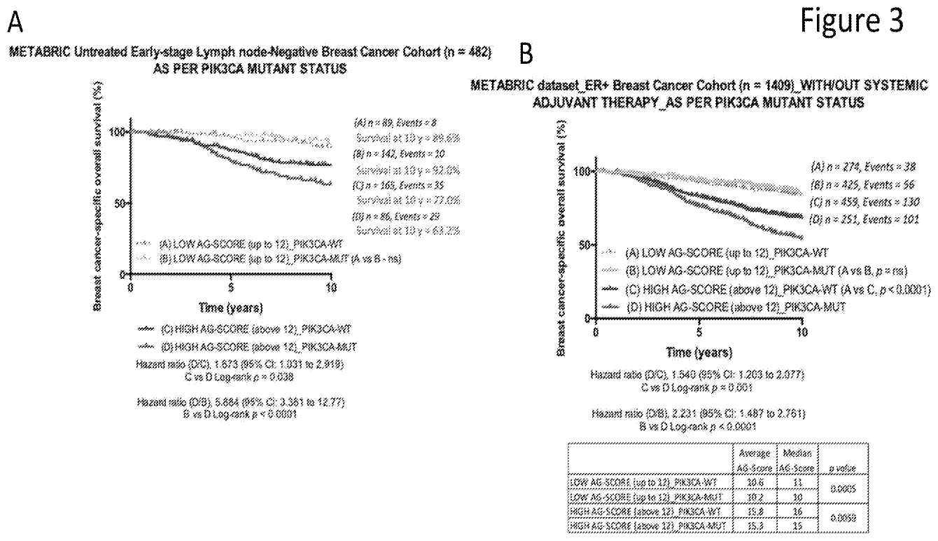

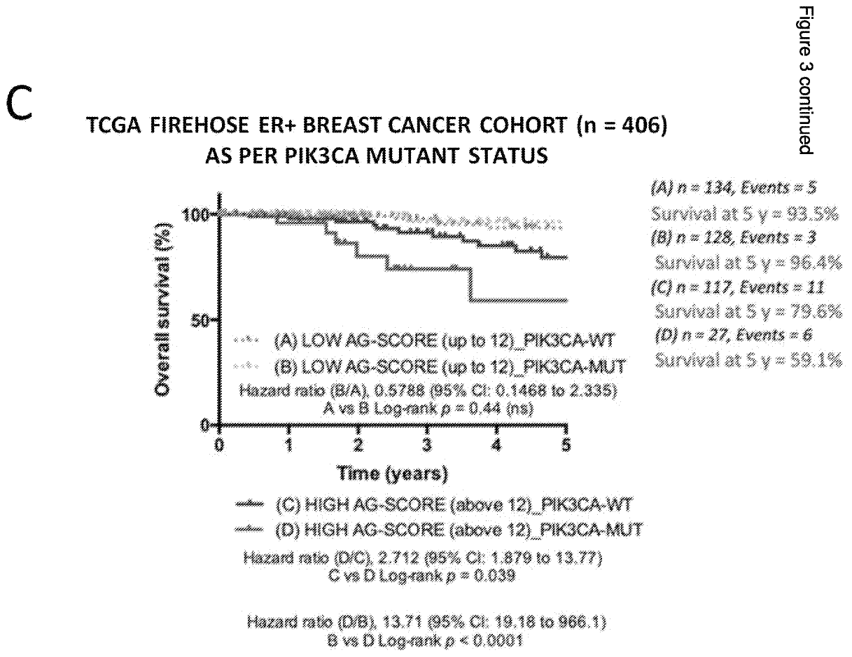

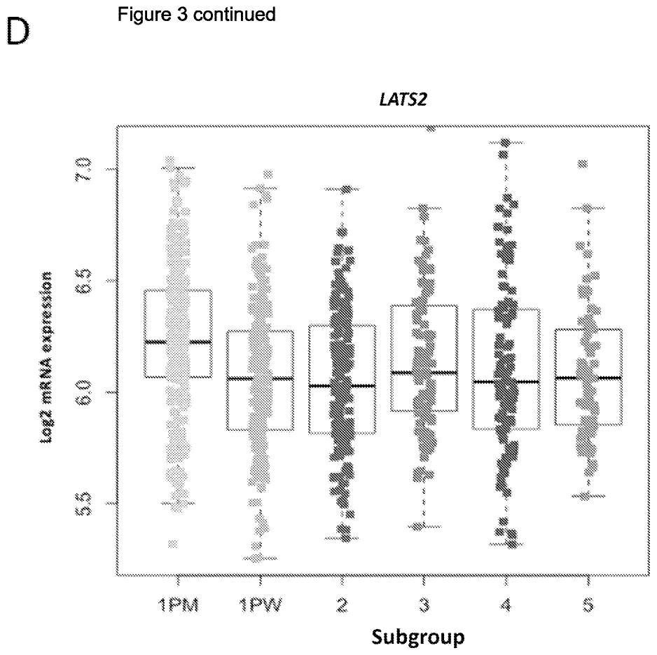

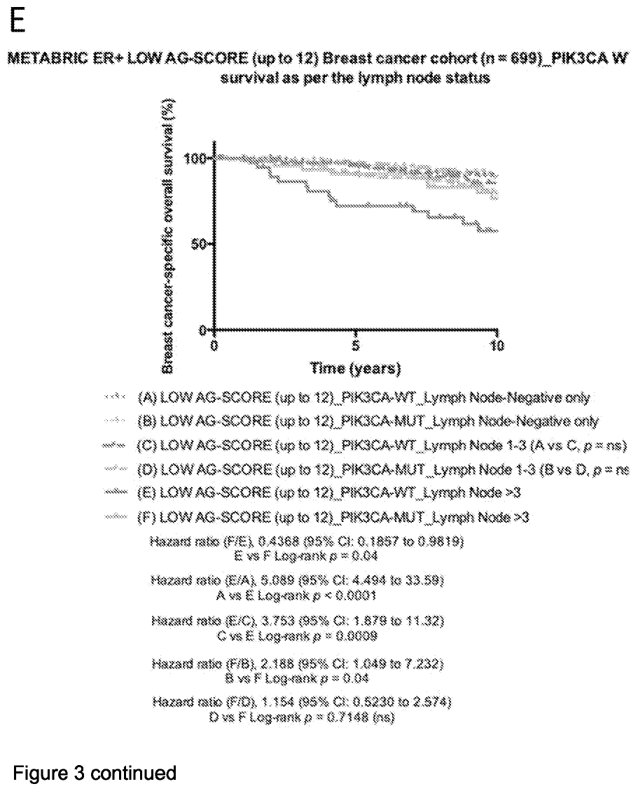

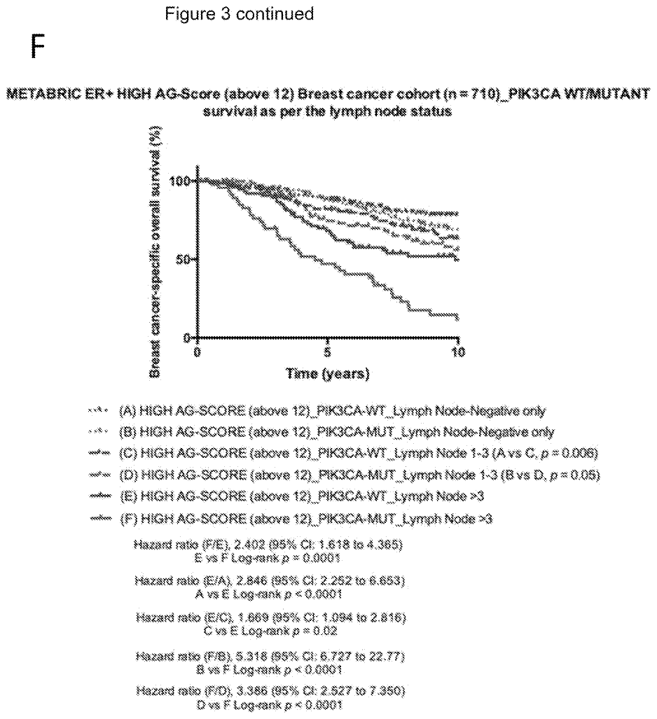

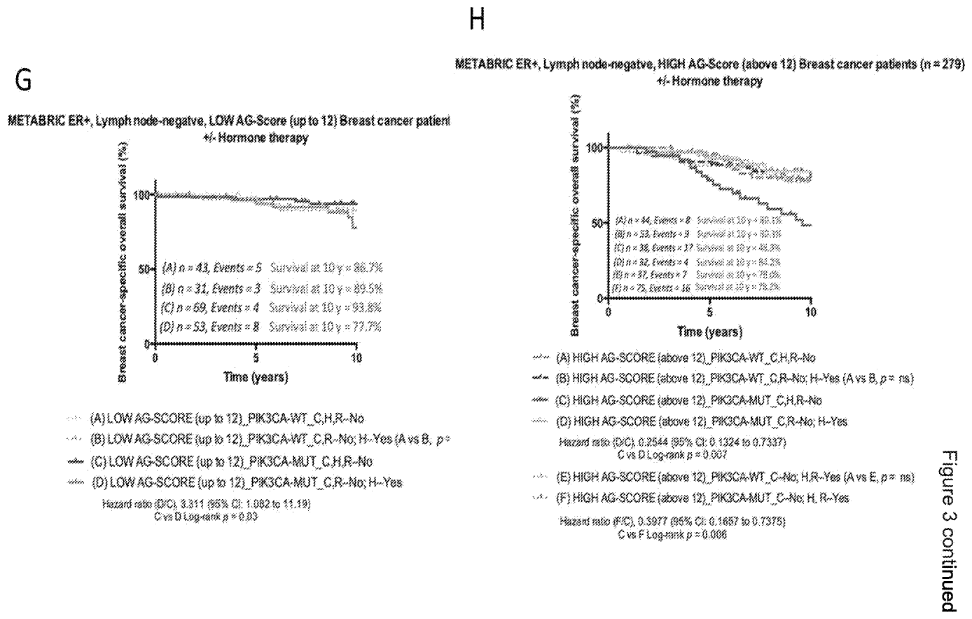

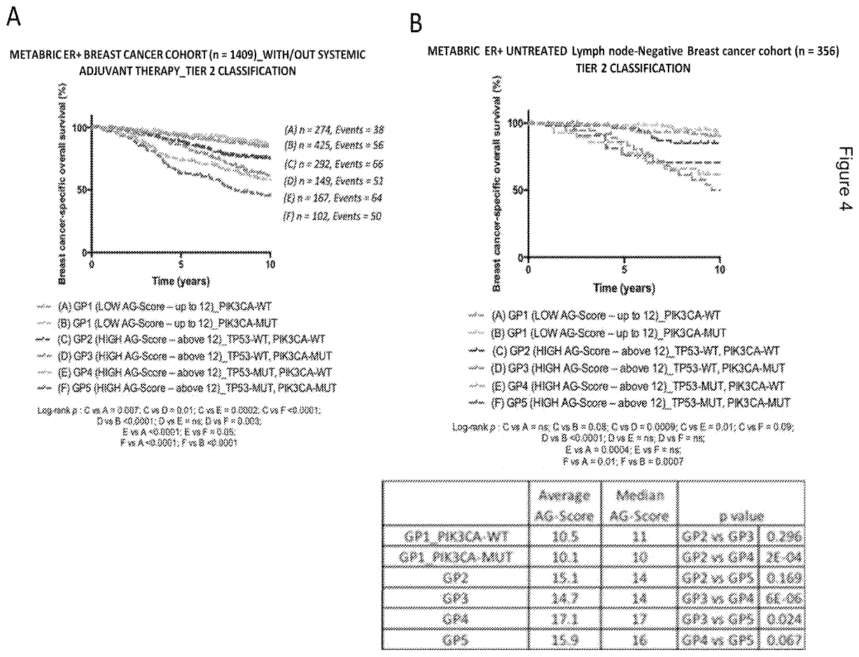

PIK3CA gene mutations, whose prognostic and predictive values are still not well understood (as discussed above), is present across the low and high-risk groups in the ER+ breast cancer cohort in the ratio of ~60:40, respectively. Remarkably, further stratification of tier-1 low and high-risk groups based on the PIK3CA gene mutations (Group B representative genomic aberration) in the early-stage (lymph node-negative) untreated breast cancer patients resulted in the identification of PIK3CA gene mutant patients who would have a good prognosis (>90% 10-year overall survival probability) as well as those with a significantly poorer prognosis (FIGS. 3A and 3C, with two different breast cancer datasets). Strikingly, the PIK3CA-mutant patients in the high-risk group (High AG-score group) had a significantly worse prognosis than the PIK3CA-WT patients in the same high-risk group, despite the former cohort having statistically significant lower AG-scores than the PIK3CA-WT patients (FIGS. 3A-3C). This highlights, first, this unique multi-level classification approach could also decipher the AG-score independent roles of breast cancer-specific aberrations, mainly attributed to their unique biologies (in this case, the unique biology associated with PIK3CA-mutant tumours, which result in a worse prognosis in the high-risk group). Second, it highlights the great clinical utility of this approach in identifying two subgroups of PIK3CA mutant patients from the early-stage (newly diagnosed) breast cancer patients, one with a good prognosis (>90% 10-year overall survival probability for early-stage untreated ER+ patients) and the second with a poor prognosis. This had not been possible until yet using the existing approaches. The prognostic values associated with PIK3CA mutations, one of breast cancer's most recurrent genomic alterations, are still poorly understood. This knowledge would be of tremendous importance in making treatment decisions for PIK3CA mutant patients and general disease management.

Moreover, it provides an excellent opportunity to comprehend the biology driving good clinical outcomes in one group of PIK3CA-mutants (Low AG-score_PIK3CA-MUT) while driving poor clinical outcomes in another group of PIK3CA-mutant (High AG-score_PIK3CA-MUT) breast cancer patients.

Analysing upregulated differentially expressed genes (DEGs) using GSEA in the PIK3CA mutant tumours associated with a good clinical outcome (i.e., Low AG-score_PIK3CA-MUT) compared to PIK3CA mutant tumours associated with poorer clinical outcomes (i.e., High AG-score_PIK3CA-MUT) revealed significantly enriched pathways for upregulated DEGs in the Low AG-score_PIK3CA-MUT tumours (and significantly enriched pathways for downregulated DEGs in High AG-score_PIK3CA-MUTs). This included pathways like Hippo signalling, TGF-beta signalling pathways. Moreover, analysis of upregulated DEGs in Low AG-score_PIK3CA-MUT versus the Low AG-score_PIK3CA-WT breast tumours also revealed the Hippo signalling pathway to be enriched in the Low AG-score_PIK3CA-MUT tumours.

Hippo signalling is an evolutionarily conserved signalling pathway that controls organ size from flies to humans. In humans, the pathway consists of the MST1 and MST2 kinases, their cofactor Salvador and LATS1 and LATS2. In response to high cell densities, activated LATS1/2 phosphorylates the transcriptional coactivators YAP and TAZ, promoting its cytoplasmic localisation, and leading to cell apoptosis and contact inhibition, restricting organ size overgrowth. When the hippo pathway is inactivated, YAP/TAZ translocates into the nucleus to bind to the transcription enhancer factor (TEAD/TEF) family of transcriptional factors to promote cell growth and proliferation. A YAP1-LATS2 feedback loop has been suggested to act as a homeostatic rheostat for dictating senescent or malignant fate, where a lack of functional LATS2 in YAP1-hyperactivated cells has been suggested to result in malignant transformation. Notably, LATS2 mRNA levels were significantly higher in PIK3CA mutant tumours associated with a good clinical outcome (i.e., Low AG-score_PIK3CA-MUT) than the High AG-score_PIK3CA-MUT or Low AG-score_PIK3CA-WT breast tumours (or it could be said that LATS2 loss in the High AG-score_PIK3CA-MUT leads to the inactivation of Hippo pathway that result in a poorer prognosis in High AG-score_PIK3CA-MUT patients) (FIG. 3D). Consistent with this, the GSEA and RPPA analysis further revealed the enrichment of critical tumour growth-supporting pathways in High AG-score_PIK3CA-MUT breast tumours (compared to Low AG-score_PIK3CA-MUT breast tumours), which include cell cycle, DNA replication, RNA transport, DNA repair, biosynthesis of amino acid, spliceosome and ribosome-biogenesis-associated molecular pathways, among others. Also, when the High AG-score_PIK3CA-MUT tumours were compared to High AG-score_PIK3CA-WT tumours, the cell cycle pathways were enriched in High AG-score_PIK3CA-WT patients (consistent with these tumours having a significantly higher AG-scores), remarkably, it is the High AG-score_PIK3CA-MUT breast patients that have a significantly worse prognosis (as also discussed above, FIGS. 3A-3C). This highlights the importance of having prognostic biomarkers based on the disease's intrinsic biology and not based on biological characteristics, such as proliferation marker Ki67 expression.

Lymph node involvement has long been recognised as the most important prognostic factor in breast cancer. Breast cancer that has spread to lymph nodes has a higher risk of returning and a less favourable prognosis than breast cancer that has not spread to the lymph nodes. The association between lymph node involvement and survival has been previously demonstrated, and it has been shown that overall survival rates are up to 40% lower in node-positive patients compared with node-negative ones. And thus, the number of lymph nodes involved (pN) has traditionally been used for post-surgical breast cancer staging. Moreover, the combination of the primary tumour (T), regional lymph nodes (N) and metastases (M) is the cornerstone of several breast cancer staging approaches, such as the breast cancer staging system of the American Joint Committee on Cancer (AJCC).

Remarkably, unlike the negative association seen with the lymph involvement in Low AG-score_PIK3CA-WT breast tumours (i.e., the prognosis gets worse with lymph node involvement in these patients), no such similarly significant effect on prognosis was observed in Low AG-score_PIK3CA-MUT breast patients (FIG. 3E). This is consistent with the above observations that the hippo pathway (with roles in contact inhibition and restricting organ size overgrowth) is activated in Low AG-score_PIK3CA-MUT breast tumours, which could be why these patients have a favourable prognosis. Notably, in the low-risk group, the lymph node-positive (with >3 node involvement) PIK3CA-MUTs have a significantly better prognosis than PIK3CA-WT patients with similar lymph node involvement (FIG. 3E). However, on the contrary, in the high-risk group (from Tier-1 classification), both PIK3CA-WT and PIK3CA-MUT breast patients showed a negative association with lymph involvement (FIG. 3F). However, in the high-risk group, PIK3CA-MUTs showed a significantly less favourable prognosis than the PIK3CA-WT breast cancer patients, unlike Low-risk patients (FIG. 3F).

Further, it is shown that identified subgroups respond differently to various therapies. For example, as illustrated in FIGS. 3G and 3H, while High risk_PIK3CA-WT and Low AG-score_PIK3CA-WT breast patients do not appear to benefit from hormone therapy, the High risk_PIK3CA-MUT breast patients respond to systemic hormone therapies (highlighting an estrogen-dependent growth in this group). Notably, it was seen that hormone therapy has a significant adverse effect on the clinical outcome of ER+, lymph node-negative Low AG-score_PIK3CA-MUT breast cancer patients (FIG. 3G), as shown by a significantly worse survival of these patients with hormone therapy. The results suggest hormone therapy should be discouraged for the Low AG-score_PIK3CA-MUT breast cancer patients (currently, in clinical settings, all early-stage ER+ are prescribed hormone therapies). Overall, this shows the clinical utility of this classification approach in identifying the correct subset of patients for any particular treatment. With earlier known methods, it had not been possible to identify subsets of PIK3CA mutated patients from the newly diagnosed early-stage breast cancer patients that would have different biology's and respond differently to various therapies (including hormone therapy).

PIK3CA mutations in breast cancer have been recently shown to be weakly associated with the Akt pathway activation (with no classification applied) (Amir Sonnenblick et al., NPJ, 2019). Data with breast cancer in vitro models also shows the same positive association of Akt pathway activation with PIK3CA alterations. The classification approach used herein shows no statistically significant effect on Akt pathway activation between the Low AG-score_PIK3CA-MUT and High AG-score_PIK3CA-MUT breast tumours. However, the RPPA analysis revealed a significantly decreased Akt pathway activation in High AG-score_PIK3CA-WT tumours than in Low AG-score_PIK3CA-WT breast tumours. This highlights that the observed positive association of PIK3CA mutations with Akt pathway activation (with no classification applied) could be related to the overall low Akt pathway activation level in PIK3CA-WT tumours.

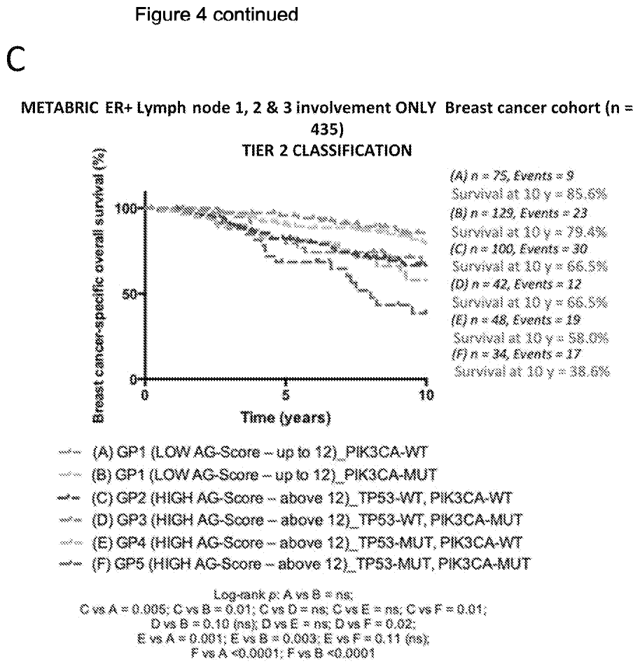

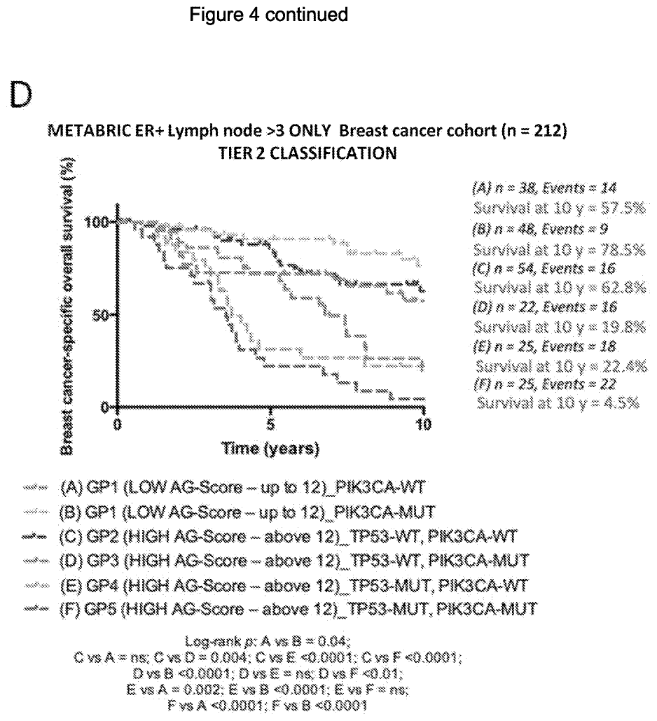

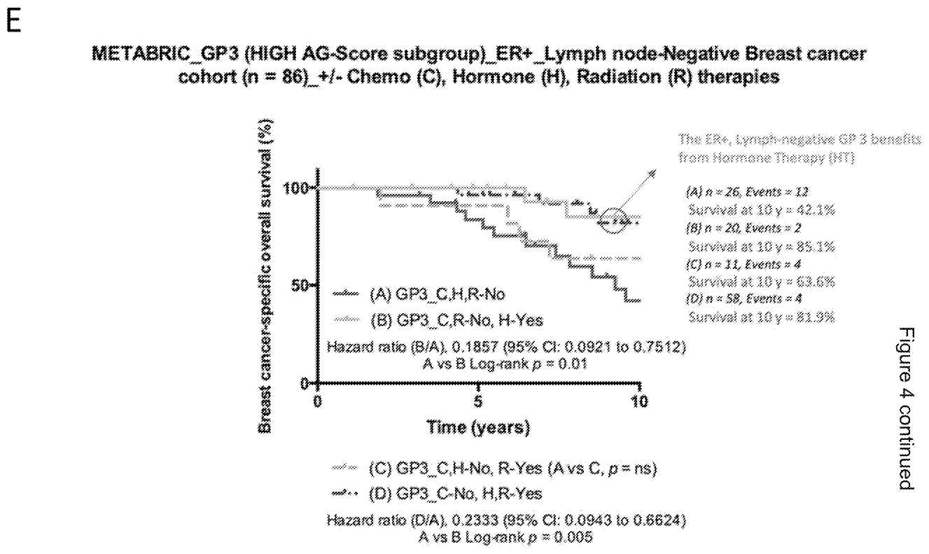

To further gain insights into these counterintuitive findings, another way of classifying the high-risk group patients based on the Group A (TP53) and Group B (PIK3CA) genomic aberrations was adopted. The high-risk group (High AG-score groups—from tier-1 classification step) were further subclassified into four subgroups with the following genomic profiles—Subgroup 2 (TP53-WT, PIK3CA-WT), Subgroup 3 (TP53-WT, PIK3CA-MUT), Subgroup 4 (TP53-MUT, PIK3CA-WT) and Subgroup 5 (TP53-MUT, PIK3CA-MUT). First, as illustrated in FIG. 4A, all four identified subgroups had unique prognoses in the ER+ breast cancer cohort (where some patients were treated with adjuvant hormone therapy or chemotherapy and/or postoperative radiotherapy), with subgroup 2 (TP53-WT, PIK3CA-WT) having a significantly favourable prognosis than the other three high-risk subgroups. This indicates that these identified subgroups are prognostic of recurrence/overall survival in early/late-stage, systemically/locally treated, and untreated breast cancer. Further, in the early-stage (lymph node-negative) untreated ER+ breast cancer cohort, Subgroup 3 (TP53-WT, PIK3CA-MUT) and Subgroup 5 (TP53-MUT, PIK3CA-MUT), despite not having a statistically significant difference in the average AG-scores than the Subgroup 2 (TP53-WT, PIK3CA-WT), have a poorer prognosis than the latter (FIG. 4B), highlighting the AG-score independent roles of subgroups, mainly attributed to their unique biology's. In the lymph node-positive (with one or more lymph-node involvement) ER+ breast cancer cohort, Subgroup 5 (TP53-MUT, PIK3CA-MUT) had a significantly worse prognosis than Subgroup 3 (TP53-WT, PIK3CA-MUT) patients (despite both having PIK3CA gene mutations in common and earlier were grouped as High-risk_PIK3CA-MUT cohort). This shows a further prognosis refinement with this second-level classification (Tier-2) from the Tier-1 classification. Furthermore, the first and second-generation commercially available prognostic multi-gene assays have so far demonstrated prognostic ability in lymph node-negative breast cancers only. However, as illustrated in FIGS. 4C-4D, the present classification approach also identifies prognostic subgroups in lymph node-positive ER+ breast cancer patients (those with >3 and those with 1-3 lymph node involvement). For example, in ER+ patients with >3 lymph node involvement, the classification approach herein identifies subgroups with varied prognoses—some significantly better than the other subgroups, with good ones having 10-year survival probability as high as 79% (Subgroup 1_PIK3CA-MUT) and poor ones (Subgroup 5—TP53-MUT, PIK3CA-MUT) with just 4.5% 10-year survival probability.

It was further investigated if the identified subgroups are predictive of hormonal therapy response. Only the Subgroup 3 (TP53-WT, PIK3CA-MUT) breast cancer patients showed improvement in overall breast cancer-specific survival with hormone therapy (FIG. 4E). No statistically significant improvements in overall disease outcome are seen in Subgroup 2, Subgroup 4, and Subgroup 5 (despite Subgroup 5 having PIK3CA gene mutations in common with Subgroup 3. This underlines an oestrogen-dependent growth in Subgroup 3 only—a further refinement from tier-1 classification where High risk_PIK3CA-MUT breast patients, in general, were predictive of hormonal therapy response). This also highlights the AG-score independent roles of each Tier-2 subgroup, mainly attributed to their unique biology's.

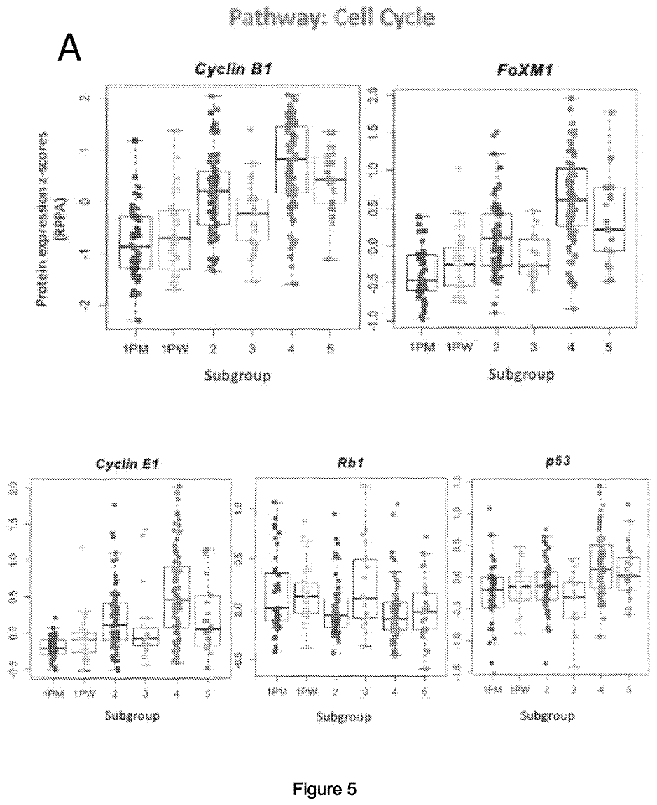

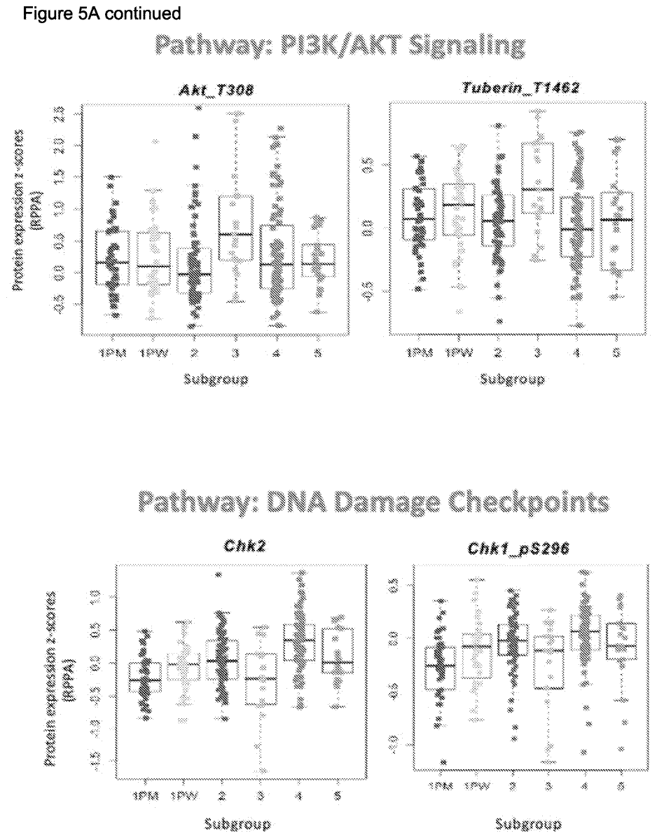

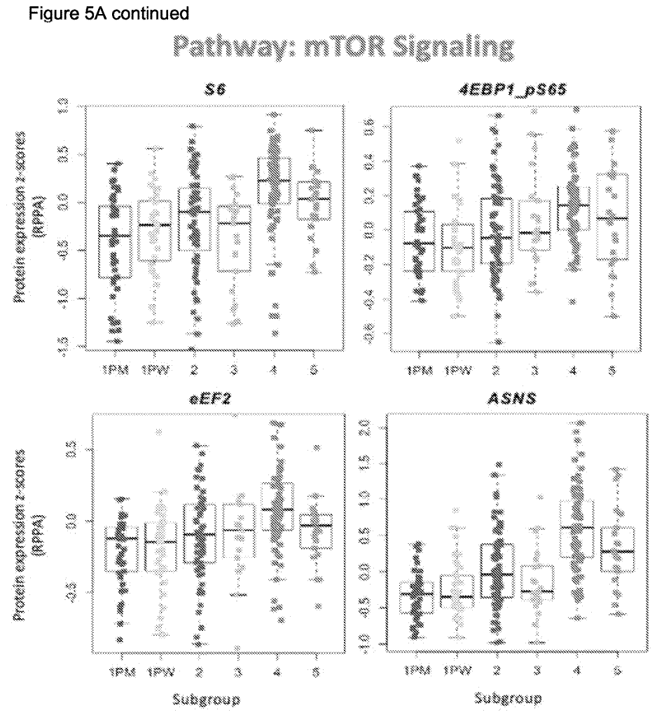

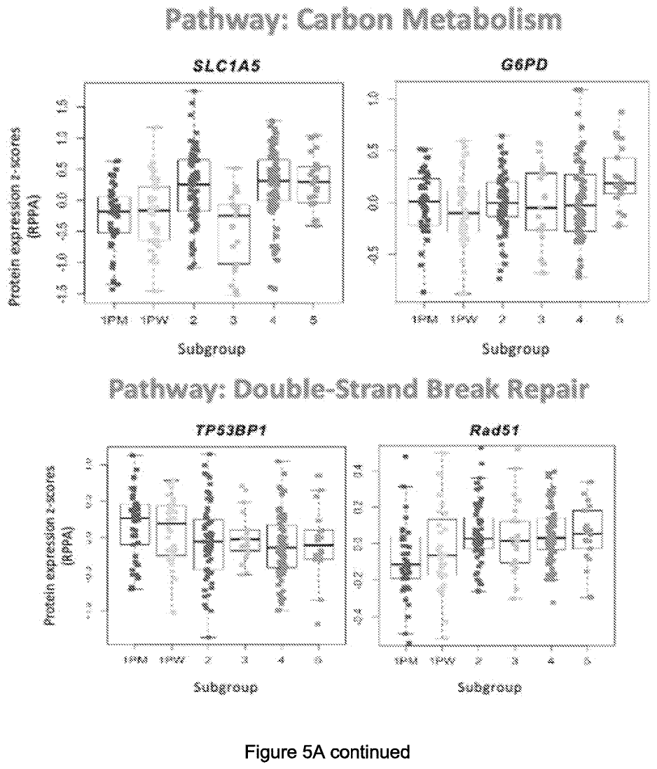

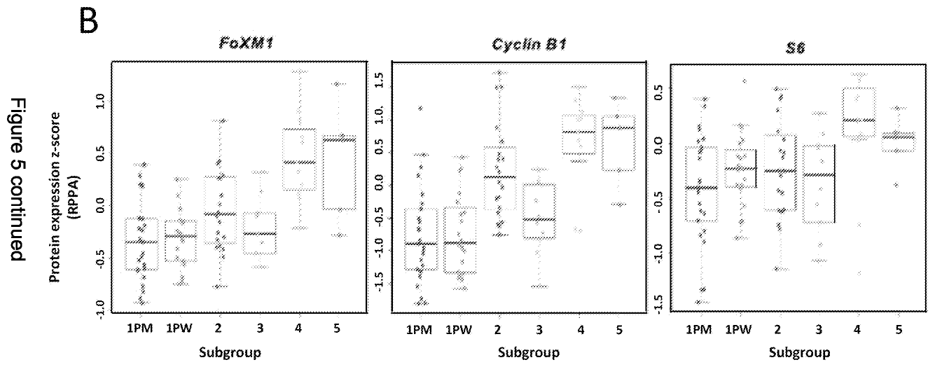

Remarkably, the RPPA analysis of these tier-2 subgroups identified the PIK3CA mutant genotype breast cancer patient subset positively associated with the PI3K/Akt pathway activation (FIG. 5A). This PIK3CA mutant genotype breast cancer patient subset belongs to Subgroup 3 (TP53-WT, PIK3CA-MUT). Of note, Subgroup 5 (TP53-MUT, PIK3CA-MUT) and Subgroup 1_PIK3CA-MUT breast tumours, despite having the PIK3CA mutant genotypes, are not associated with the Akt pathway activation. As noted above, the PIK3CA-WT subgroups—Subgroup 2 (TP53-WT, PIK3CA-WT) and Subgroup 4 (TP53-MUT, PIK3CA-WT), showed a significantly decreased Akt pathway activation than Subgroup 1_PIK3CA-WT breast tumours.

Most PIK3CA mutations are missense mutations positioned in the helical domain (exon 9, mostly: E545K and E542K) and the kinase domain (exon 20, mostly H1047R) in hotspot clusters (15). These mutations have a direct effect on AKT phosphorylation/activation. The RPPA analysis revealed that mutations in the PIK3CA kinase domain are more robustly associated with PI3K/AKT pathway activation than other exons, specifically in Subgroup 3 (TP53-WT, PIK3CA-MUT) breast cancer patients (FIG. 5E).

Recent exciting clinical trial results in advanced ER+ breast cancer support mTOR activation as a major means of estrogen-independent tumour growth. Hence the means to identify a responsive breast cancer population that would most benefit from these compounds in the adjuvant or earlier stage setting is of high interest. Notably, the RPPA analysis (FIGS. 5A and 5B) further identified subgroups positively associated with the mTOR pathway activation—belonging to Subgroup 4 (TP53-MUT, PIK3CA-WT) and Subgroup 5 (TP53-MUT, PIK3CA-MUT). Subgroup 2 (TP53-WT, PIK3CA-WT) showed a moderate mTOR pathway activation.

While AKT is activated by phospholipid binding and activation loop phosphorylation at Threonine308 by PDK1 and by phosphorylation within the carboxy terminus at Serine473, mTOR is activated via the PI3K-signaling pathway (7) AKT activates the mTOR complex 1 (mTORC1) which in addition to mTOR contains mLST8, PRAS40, and RAPTOR. This activation involves phosphorylation of tuberous sclerosis complex 2 (TSC2), which blocks the ability of TSC2 to act as a GTPase-activating protein, thereby allowing accumulation of Rheb-GTP and mTORC1 activation.

The PI3K/AKT/mTOR pathway is usually considered a linear signal transduction pathway in breast cancer. However, as discussed above, in the ER-positive disease, it has been previously shown that PIK3CA mutations, in general, were associated with relatively low mTORC1 functional output and with good outcomes in patients. How the PIK3CA mutation contributes to breast cancer growth, and, most importantly, why robustly high levels of classical PI3K/AKT/mTOR signalling are not observed in human breast cancers, had been an open question to date. This may be crucial to understanding who will respond to therapeutic PI3K or mTOR pathway inhibition.

Considerable attention has been devoted to the search for molecular predictive biomarkers of response to PI3K/AKT pathway inhibitors. As discussed above, particularly for the ER+ or “luminal” BCs, PIK3CA mutations occur commonly and are thought to represent a logical target population for this therapy. However, several issues have complicated PIK3CA mutations as a predictive biomarker in BC: (i) PIK3CA mutations have been associated with a relatively better prognosis compared with PIK3CA wild-type BC patients, (ii) the mutations are not associated with higher proliferation indices or lower efficacy with hormonal therapy which would be hypothesized from oncogenic activation of PIK3CA [8], and (iii) PIK3CA mutant breast cell lines have been associated with sensitivity to tamoxifen. Hence the effect of PIK3CA mutational activation on signaling and clinical relevance in ER-positive BC had been unclear until now.

Overall, the present findings are highly significant as the current state of the art does not provide tumour biology insights with as great detail and has been chiefly confounded with counterintuitive/paradoxical findings in a clinical setting. Notably, the present PIK3CA-mutant genotype breast cancer example illustrates how the present classification approach helps understand the nuance, context and significance of cancer genomic aberrations. For example, despite the Subgroup 1_PIK3CA-MUT, Subgroup 3 (TP53-WT, PIK3CA-MUT) and Subgroup 5 (TP53-MUT, PIK3CA-MUT) breast cancer patients having PIK3CA gene mutations in common, it is only Subgroup 3 breast cancer patients that show a robust PI3K/AKT pathway activation and thus should respond to therapeutic PI3K inhibition based on this Tier-2 classification approach. The present invention also clarifies the clinical relevance of PIK3CA mutational activation (subgroup-specific) in breast cancer.

Understanding the precise tumour biology may have critical clinical implications considering that in low mTOR breast cancers, treatment with mTOR inhibitors, such as everolimus, which is highly toxic, will conceivably be of lower value since the pathway is not activated and thus might do more harm than providing any benefit. Additionally, Akt pathway activation may be critical to understanding who will respond to therapeutic PI3K inhibition. It can add to presently used outcome prediction tools (mostly PIK3CA mutation biomarker-based, i.e. if the breast cancer patient carries PIK3CA mutation).

Further, besides providing insights into the PI3K/AKT/mTOR signalling pathway activation, the method described here also provides tumour biology insights about other molecular pathways (FIGS. 5A-5C) in hormone receptor-positive/negative, lymph node-negative and/or -positive breast cancer patients. Each identified subgroup is characterised by a unique set of biomarkers that could guide treatment decisions leading to improved quality of treatment and better therapy response for cancer patients (FIGS. 5A-5C). The provided subgroup-specific tumour biology insights are agnostic to the technology used to quantify RNA expression data.

Notably, the RPPA analysis of breast cancer patients with >3 lymph node involvement shows tumour biology in Subgroup 1_PIK3CA-MUT patients highly consistent with the observation of hippo pathway activation and favourable prognosis in this cohort discussed above (FIG. 5D). Subgroup 1_PIK3CA-MUT patients with >3 lymph node involvement do not show activation of pathways/proteins involved in tumour growth, such as cell cycle pathways (FIG. 5D). For example, levels of cell cycle biomarkers, cyclin B1 or Forkhead box transcription factor (FoxM1), are markedly lower in Subgroup 1_PIK3CA-MUT patients than the rest of the subgroups in breast cancer patients with >3 lymph node involvement (FIG. 5D). Furthermore, clinical outcome analysis of 180 breast cancer patients who developed metastatic breast cancer (from a separate cancer dataset, The Metastatic Breast Cancer Project) further validates the above findings with Subgroup 1_PIK3CA-MUT patients. The analysis reveals Subgroup 1_PIK3CA-MUT patients to have a significantly favourable prognosis than any other subgroups (taking an average of 1750 days from primary disease diagnosis to metastatic disease diagnosis, whereas the rest of the subgroups take on average <500 days). These results are highly consistent with the findings discussed above, emphasising a favourable prognosis and tumour biology of Subgroup 1_PIK3CA-MUT breast cancer patients (with or without lymph node involvement) because of the active hippo pathway in this patient subset.

Alpelisib (PIQRAY®) is a novel PI3K pathway inhibitor drug recently approved for clinical use in HR-positive, HER2-negative, locally advanced or metastatic breast cancer patients, specifically with a P/K3CA mutation (Alpelisib drug is currently not approved for early-stage breast cancer). However, as the present finding suggests, not all PIK3CA-mutant breast tumours are biologically identical (even in advanced breast cancer settings). This highlights the severe shortcomings of the current single-gene biomarker-based precision cancer medicine approaches (based on PIK3CA mutation status in the case of Alpelisib). The present classification approach allows an understanding of the nuance, context and significance of genomic aberrations in the genomically complex landscape of tumours. For example, as per the current classification approach, the Subgroup 1_PIK3CA-MUT and Subgroup 5 (TP53-MUT, PIK3CA-MUT) advanced breast cancer patients would not benefit from PI3K pathway inhibitor drugs. Subgroup 3 (TP53-VVT, PIK3CA-MUT) PIK3CA mutant breast patients show a robust PI3K/AKT pathway activation and thus would benefit from such therapies. As the present findings highlight, Subgroup 3 (TP53-WT, PIK3CA-MUT) PIK3CA-mutant breast patients showed estrogen-dependent growth with high PI3K/AKT pathway activation; thus, a combination of hormone therapy with PI3K pathway inhibitor drug would benefit these patients (irrespective of the stage of the disease). This implies that early-stage Subgroup 3 breast cancer patients would also benefit from hormone therapy and PI3K pathway inhibitor drug combination treatment, thus, identifying new cohorts that would benefit from targeted therapy.

Moreover, consistent with our subgroup-wise RPPA findings on mTOR pathway activation (FIGS. 5A and 5B), a high number of responders to the mTOR inhibitor, Everolimus is observed in High AG-Score_PIK3CA-WT subgroups comprising Subgroups 2 & 4 (a total of 23 responders out of 23 breast cancer patients). However, in the Low AG-Score_PIK3CA-WT subgroups, the majority were non-responders—8 non-responders out of 10 patients. These findings again highlight how the present classification approach allows an understanding of the nuance, context and significance of genomic aberrations in the genomically complex landscape of tumours and would be critical in identifying patient subsets responding to specific therapies. Each subgroup identified through the present classification approach provides unique insights into the tumour biology and molecular pathways driving cancer in that subgroup. This information is highly useful for guiding treatment decisions.

As demonstrated earlier (at the tier-1 classification level), statistical approaches (GSEA, over-representation analysis and/or pathway/network enrichment analyses, etc.) are used to gain further insights into tier-2 subgroup-specific biology's by identifying gene sets that share a common biological function, chromosomal location, or regulation. The statistical approaches, such as GSEA, can compare different subgroups in multiple ways to identify driver molecular pathways at the subgroup-specific level. For example, one way is by comparing a subgroup, e.g., Subgroup 2 (TP53-WT, PIK3CA-WT) ER+ breast tumours, with the rest of the subgroups (i.e. Subgroup 1, Subgroup 3, Subgroup 4 and Subgroup 5 ER+ breast tumours). This involves computing the differentially expressed genes (DEGs) between Subgroup 2 tumour samples and the rest of the remaining subgroups' samples and performing an over-representation/enrichment analysis (which can be done separately for up- and down-regulated genes), and identifying enriched/over-represented molecular pathways.

Another way involves comparing a subgroup, e.g., Subgroup 2 (TP53-WT, PIK3CA-WT), individually with other subgroups, i.e. Subgroup 1 (as a whole or just the Subgroup 1_PIK3CA-WT), Subgroup 3, Subgroup 4 and Subgroup 5, and performing an over-representation/enrichment analysis using a union of all DEGs from each analysis. The analysis highlights the molecular subgroup-specific distinct and exclusive pathways driving cancer in each subgroup and the common pathways (with the overlapping/shared gene sets representing a gene signature for that molecular subgroup. These gene signatures highlight an alternative way to stratify breast tumours into respective tier-2 molecular subgroups). It also allows the identification of how each subgroup differs from the other and provides an opportunity to rank the degree of any specific molecular pathway’ activation among the subgroups. For example, regarding the enrichment of the cell cycle pathway, one-on-one GSEA between the subgroups revealed the following order of the gene sets belonging to the cell cycle pathway being enriched: Subgroup 4 and Subgroup 5>Subgroup 2>Subgroup 3>Subgroup 1_PIK3CA-WT>Subgroup 1_PIK3CA-MUT.

The differential gene expression analysis between the high-risk (High AG-Score) molecular subgroups and the low-risk subgroups (as a whole Subgroup 1 or just the Subgroup 1_PIK3CA-WT or Subgroup 1_PIK3CA-MUT) can be used to decipher the mechanism(s) of tumour evolution (i.e. molecular pathways driving progression from low-risk to high-risk cancer). E.g., comparing Subgroup 2 with Subgroup 1_PIK3CA-WT, Subgroup 3 with Subgroup 1_PIK3CA-MUT, Subgroup 4 with Subgroup 1_PIK3CA-WT, Subgroup 5 with Subgroup 1 and finding the shared DEGs (intersection/overlapping DEGs) from each analysis. This shared DEGs list acts as a collective gene set (gene signature) representing genes involved in the progression of low-risk breast cancer to high-risk cancer. The list describes the gene signature for tier-1 low-risk (low AG-score) and high-risk (high AG-score) group classification. The shared (intersection/overlapping) up-regulated DEGs between the high-risk molecular subgroups had 1 marked as the direction of the association, implying a negative correlation with an increased likelihood of a good clinical outcome (high-risk). Whereas the shared (intersection/overlapping) down-regulated DEGs between the high-risk molecular subgroups had a −1 direction of the association, implying a positive correlation with an increased likelihood of a good clinical outcome (low-risk). This indicates that a higher expression of an RNA transcript from the shared (intersection/overlapping) up-regulated DEGs between the high-risk molecular subgroups with its direction of association marked 1 (up-regulated in the high-risk molecular subgroups) would correlate with a decreased likelihood of a good clinical outcome (corresponding to a high-risk tier-1 group). Conversely, a higher expression of an RNA transcript from the shared (intersection/overlapping) up-regulated DEGs between the high-risk molecular subgroups with its direction of association marked −1 (down-regulated in the high-risk molecular subgroups) would correlate with an increased likelihood of a good clinical outcome (corresponding to a low-risk tier-1 group).

Like the gene signatures described above as an alternative way to stratify breast tumours into respective tier-2 molecular subgroups, proteogenomic characterisation and protein markers were found to discriminate between molecular groups as well (FIGS. 5A-5D). For example, some proteins were highly enriched in various tier-2 molecular groups: XRCC1 and Cyclin D1 for Subgroup 2, Akt_pT308 and Tuburin_pT1462 for Subgroup 3, mTOR and cell cycle protein gene sets (e.g., S6, 4EBP1_pS65, FoxM1, Cyclin B1, etc.) for Subgroup 4 and G6PD for Subgroup 5. These results show the potential for molecular group classifications to be adopted in conventional pathology laboratories using immunohistochemistry approaches, for example.

Tier-3 classification—The biology of cancer is complex. It involves the tumour microenvironment and cross-talk between various signalling pathways, including the immune system. The immune component of the tumour microenvironment is now widely recognised as a hallmark of cancer. An overwhelming amount of data from animal models and compelling data from human patients indicates that a functional cancer immunosurveillance process can act as an extrinsic tumour suppressor.

“Hot” tumours describe a tumour showing signs of inflammation (triggering a robust immune response), meaning the tumour has already been infiltrated by T cells to attack and kill the tumour cells. For this reason, hot tumours typically respond well to immunotherapy treatment using checkpoint inhibitors. The main idea behind checkpoint inhibitors is using antibodies to mobilize the T cell response. During the attack on the tumour, T cells become exhausted and lose essential functions needed to kill tumour cells. The exhaustion is brought on by constant exposure to tumour antigens and signalling through checkpoint receptors (e.g., PDL1, IDO, CTLA4). The antibodies block signalling through these receptors to prevent this loss of function. Only a few types of cancers are considered hot—including melanoma, bladder, kidney, head and neck, and non-small cell lung cancer—and further limiting the efficacy of immunotherapies is the fact that not every hot tumour will be responsive to such treatments. Also, most hormone receptor-positive breast cancers, which are the majority of breast cancers, are typically considered cold tumours, which poses a problem for the widespread use of immunotherapies in this cancer type.

Notably, immunotherapy has potential downsides. Although immunotherapy is designed to help the immune system attack cancer cells, immune cells may mistakenly attack healthy tissue leading to one or more side effects (at times immune-related severe adverse effects). Thus, it is crucial to identify robust biomarkers to identify patients who might benefit from immunotherapies. Identifying hot tumours with intrinsic capabilities to attack and kill the tumour cells (intrinsic low-risk tumours) without further requirement of any therapies or who might safely benefit from de-escalating chemotherapy and/or endocrine therapy regimens, thus avoiding overtreatment, is also vital.

The third tier of molecular classification includes subgrouping based on an immune gene signature reflecting the tumour's immune landscape. The method involves deriving an AG-immune risk score using an immune gene signature based on two or more or three or more combined groups of genes listed in Table 5. The aim was to classify tumours from the above-identified breast cancer subgroups as hot or cold tumours based on the calculated AG-immune score. Further, the method aimed to identify hot tumours having intrinsic capabilities to attack and kill the tumour cells—whereby patients with hot tumours (i.e. high AG-immune scores) that have >90% survival chances for up to 20 years post-diagnosis are considered to be low-risk and would not require of any therapies or escalating of chemotherapy and/or endocrine therapy regimens, thus avoiding overtreatment.

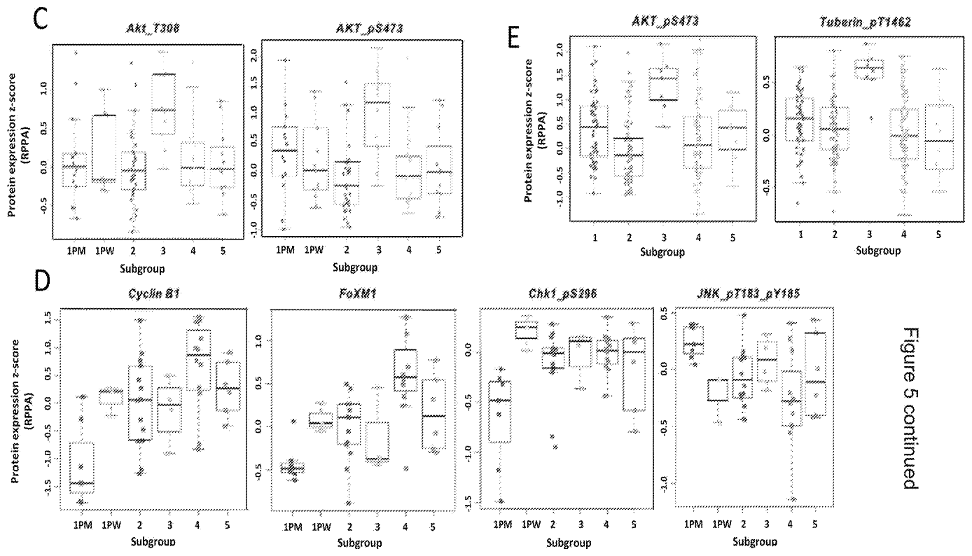

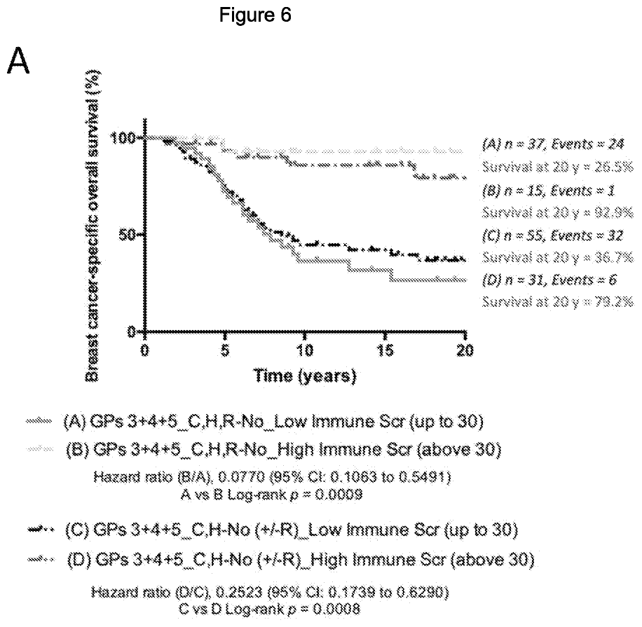

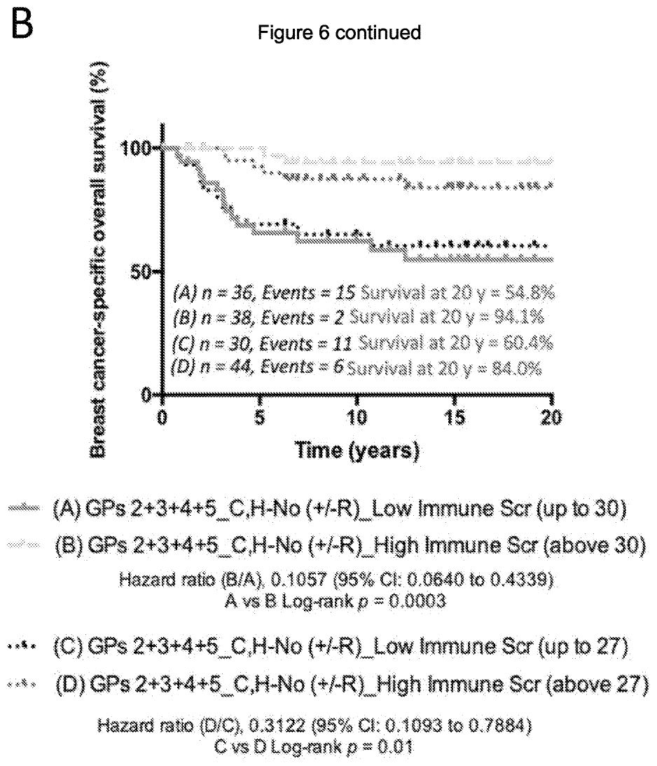

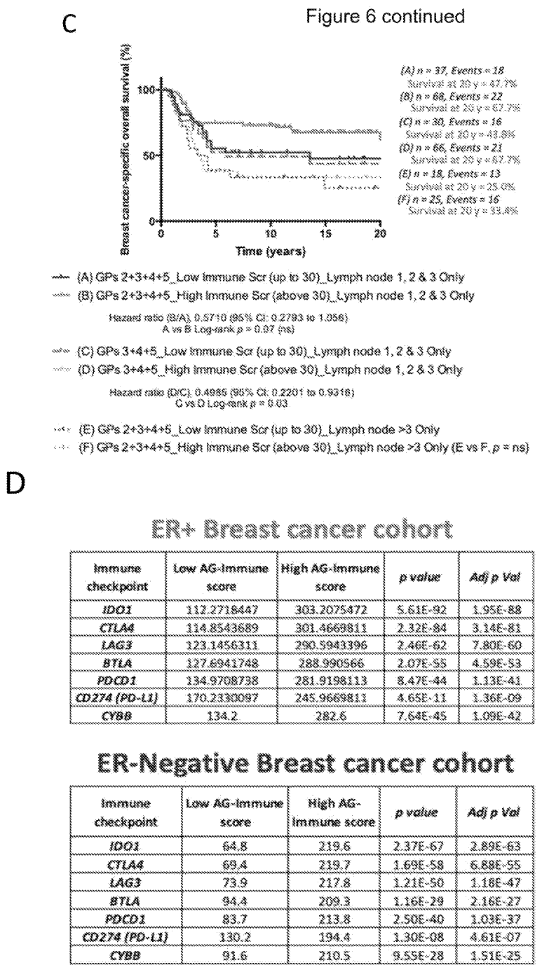

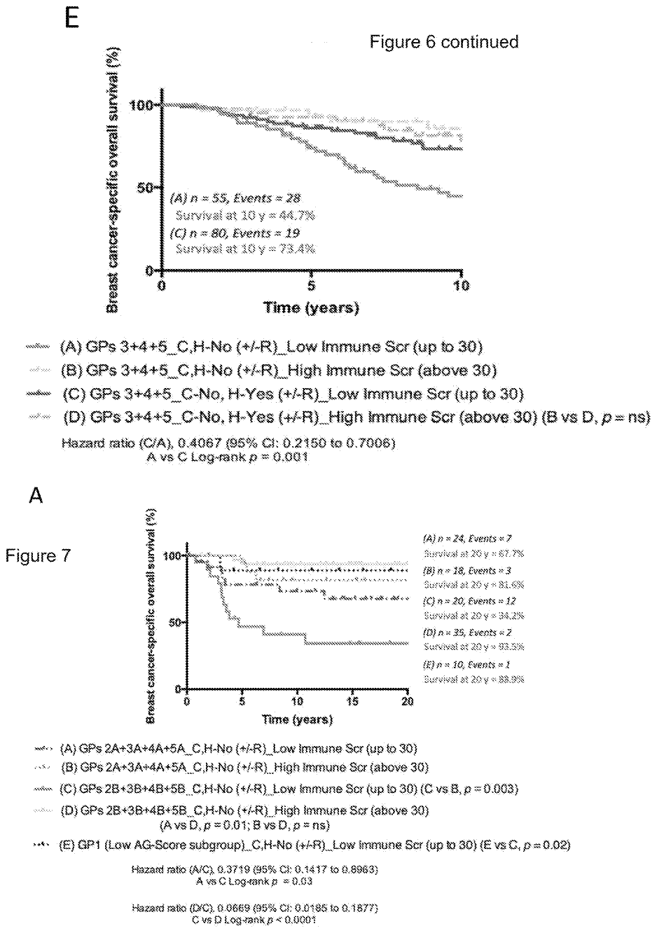

As shown in FIGS. 6A to 6C the AG-immune risk score derived from a gene signature (comprising seven combined groups of RNA transcripts/genes) from those identified as above was used to stratify further the breast cancer subgroups identified above using the current multi-tier classification approach. The provided examples encompass various breast cancer histopathological subtypes, including lymph-negative ER+, ER−, ER− HER2− (TNBC), and HER2+ untreated (patients have not received any systemic adjuvant therapy) breast cancer cohorts, as well as lymph node-positive ER+ and TNBC cohorts.

Like the AG-score (risk-score), the AG-immune risk score was assessed as a continuous function using receiver operating characteristic curves. It was found that the immune scores could be classified as low and high, respectively. Various cut-off values for AG-immune scores were used to classify a subject as having a low or high-immune risk score (as a 2-class risk model, FIGS. 6A and 6B). High/low immune scores in specific breast cancer subgroups were predictive of improved clinical indications, such as overall survival. It was found that an immune risk score is predictive (positively correlated) for subgroups' clinical outcome (in terms of overall survival, relapse/disease-free survival, recurrence-free survival, metastasis-free survival, event-free survival, etc.), like in case of lymph node-negative ER+ untreated (patients have not received any systemic adjuvant therapy) Subgroup 3 breast cancer patients. Moreover, it also identified specific breast cancer subgroups where the high/low immune scores are not predictive of clinical outcome, like in the case of lymph-negative ER+ Subgroup 2 (TP53-WT, PIK3CA-WT) breast cancer patients, as the high and low-immune risk score Subgroup 2 patients had no statistically significant difference in their clinical outcomes. Further, method identified a cluster of subgroups (from tier-2 classification) in lymph-negative ER+ untreated breast cancer cohort where the immune signature described above was prognostic and/or predictive, for example, identifying a cluster of subgroups encompassing high-risk Subgroups 3, 4 and 5 which have high immune scores (implying activation of adaptive and innate immunity—hot tumours) and characterised by good prognosis; or have low immune-scores and characterised by poor prognosis (FIG. 6A). For example, in the lymph node-negative ER- and ER− HER2− (TNBC) untreated breast cancer cohorts (early-stage breast cancer cohorts), which received no systemic adjuvant therapy, the method identified a subgroup cluster (FIG. 6B) comprising high-risk Subgroups 2, 3, 4 and 5, with high immune scores and characterised by a good prognosis (with >90% 20-year overall survival probability). Conversely, in the lymph node-negative ER- and TNBC breast cancer cohorts, which received no systemic adjuvant therapy, the method also identified, for example, a subgroup cluster comprising Subgroups 2, 3, 4 and 5, with low immune scores and characterised by a poor prognosis (with <90% 20-year overall survival probability) (FIG. 6B). Given that these subgroup clusters (from lymph-node negative ER+, ER- and TNBC breast cohorts) with high AG-immune scores (hot tumours) have >90% survival chances for up to 20 years post-diagnosis, they are considered to be low-risk. These are the patient subsets with intrinsic capabilities (i.e. having robust immune response) to attack and kill the tumour cells and thus might not need any therapies (patients from FIGS. 6A and 6B) who have not received any systemic adjuvant therapy) or escalating chemotherapy and/or endocrine therapy regimens, thus the AG-immune risk score could help avoid overtreatment. For example, it is seen that chemotherapy treatment to a lymph node-negative TNBC high immune-score subgroup cohort (comprising Subgroups 2, 3, 4 and 5) significantly worsens the overall prognosis of this cohort compared to a similar cohort that is left untreated with systemic adjuvant therapies).

Moreover, in the lymph node-positive TNBC (late-stage breast cancer cohort) and lymph node-negative HER2+ breast cancer cohorts, the high-low immune score-based stratification is prognostic and identified a subgroup cluster comprising subgroups 2,3,4 and 5, with high immune scores (hot tumours) and a significantly better prognosis than the low immune scores in the same cohort (see example FIG. 6C). The high immune risk score subgroups cluster 2,3,4 and 5, though they have a significantly better prognosis than those with low immune scores, have a 20-year overall survival probability of <90% and could make an ideal cohort who would benefit from immunotherapies/checkpoint inhibitors (FIG. 6C). This is because, usually, the expression of immune checkpoints is markedly higher in the ER+ and ER− high-immune risk score subgroups compared to the low-immune risk score counterparts (FIG. 6D)—checkpoint inhibitors would thus help mobilise the exhausted T-cell response to attack the tumour and kill tumour cells.

As shown above, the AG-immune risk score positively correlates with some individual subgroups' clinical outcomes. While higher cut-off values for AG-immune scores (e.g., an AG-immune risk score cut-off value of 30 in lymph node-negative ER+ and TNBC untreated breast cancer subgroup clusters from FIGS. 6A and 6B) can result in >90% 20-year overall survival probability for high immune risk score patients, a lower cut-off value for AG-immune scores though can have a significantly better prognosis than those with low immune scores but have a 20-year overall survival probability of <90% (e.g., an AG-immune risk score cut-off value of 27 in lymph node-negative ER+ and TNBC untreated breast cancer subgroup clusters). With the rationale provided above, the latter cohort (e.g., patients with an intermediate immune score, i.e. immune risk score between 27 and 30, from ER+ breast cancer Subgroups 3,4 and 5 clusters or TNBC Subgroups 2,3,4 and 5 clusters) could also make an ideal cohort that would benefit from immunotherapies (checkpoint inhibitors) to help mobilise the exhausted T-cell response to attack the tumour and kill tumour cells effectively. Overall, the present tumour profiling approach provides biomarkers that could be useful in identifying patient subsets that would benefit from immunotherapy.

The sub-classification of the tier-2 subgroups using an additional tier (tier-3) classification further refines the biology and prognosis of the tier-2 subgroups. In addition, this further identifies low-risk patient subgroups (with >90% 20-year overall survival probability) from otherwise high-risk patients group identified from the tier-2 classification. For example, in an untreated early ER+ breast cancer cohort (that received no systemic adjuvant therapy), the additional tier of classification (tier-3) of high-risk tier-2 subgroups 3, 4 and 5 can identify low-risk patient subgroups that have high immune scores and >90% 20-year overall survival probability. These newly recognised low-risk patient subsets (high immune-score subgroups-3, -4 and -5 in breast cancer) identified from the high-risk tier 2 subgroups 3, 4 and 5 could thus avoid overdiagnosis and overtreatments, given their >90% 20-year overall survival probability even without any systemic therapies (FIG. 6A). Similarly, in an untreated early ER- and TNBC breast cancer cohort (that received no systemic adjuvant therapy), the additional third tier of classification of high-risk tier-2 subgroups 2, 3, 4 and 5 can identify low-risk patient subgroups with high immune risk score and >90% 20-year overall survival probability. These newly recognised low-risk patient subsets (high immune-score subgroups -2, -3, -4 and -5) identified from the high-risk tier-2 subgroups 2, 3, 4 and 5 could thus avoid overdiagnosis and overtreatments (FIG. 6B). It was seen that about 48% of early-stage ER-negative patients that would not require systemic therapies based on the above findings that show >90% 20-year overall survival probability of high-immune risk score TNBC patients (even without systemic therapies).

It was further determined if the identified subgroups from the tier-3 classification could help refine the hormonal therapy response prediction value for hormone receptor-positive breast cancers. At the tier-2 classification, it was found that only the Subgroup 3 (TP53-WT, PIK3CA-MUT) breast cancer patients show improvement in overall breast cancer-specific survival with hormone therapy (FIG. 4E). Notably, the tier-3 classification can identify new subgroups in hormone receptor-positive breast cancers that would benefit from hormonal therapy. As FIG. 6E illustrates, it was seen that the Low_immune_score_Subgroup 4 and Low_immune_score_Subgroup 5 ER+ breast patients also had improved overall breast cancer-specific survival with hormone therapy (along with Low_immune_score_Subgroup 3). No statistically significant improvements in overall disease outcome are seen in Low_immune_score_Subgroup 2 with hormone therapy. This again highlights the AG-score independent roles of each Tier-3 subgroup, mainly attributed to their unique biology's’. Similarly, the tier-3 classification could help identify patient subgroups that would benefit from chemotherapy.

The Tier 3 classification can further involve using the statistical approaches (GSEA, over-representation analysis and/or pathway/network enrichment analyses, etc.) to gain insights into the tier 3 subgroups-specific biology's and interpret the specific underlying biological/molecular mechanisms driving cancer growth in these subgroups and understand the molecular/genetic processes that lead to the development of tumours with reduced immunogenicity's and identify the molecular targets of the cancer immunoediting process to gain insight into how tumour sculpting can be prevented.

As demonstrated earlier (at the tier-1 and tier-2 classification levels), statistical approaches (GSEA, over-representation analysis and/or pathway/network enrichment analyses, etc.) can provide further insights into tier-3 subgroup-specific biology's by identifying gene sets that share a common biological function, chromosomal location, or regulation. Statistical approaches, such as GSEA, were used in multiple ways to compare different subgroups and identify subgroup-specific driver molecular pathways. The enriched molecular pathways in each tier-3 subgroup (that defines each subgroup) from an ER+ breast cancer cohort can be determined as described above for tier-2 classification. The analysis highlighted the molecular subgroup-specific distinct and exclusive pathways driving cancer in each subgroup and the common pathways (with the overlapping/shared gene sets representing a gene signature for that molecular subgroup). This highlights an alternative way to stratify breast tumours into respective molecular subgroups.

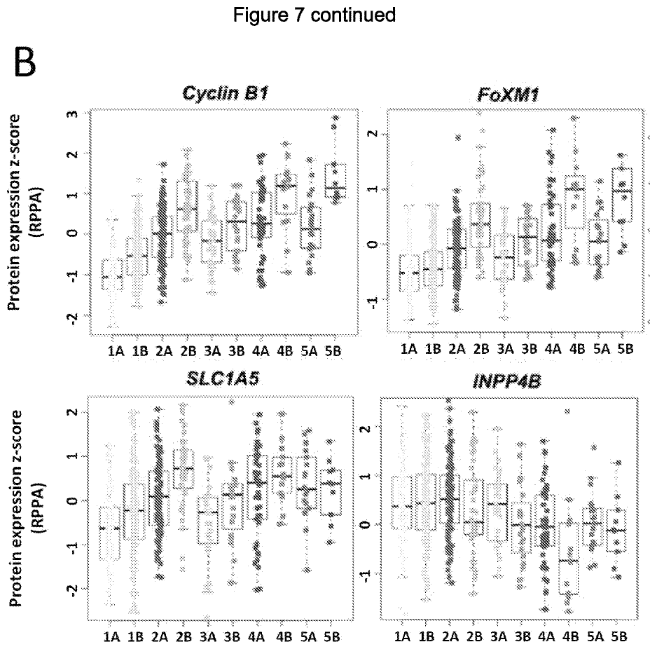

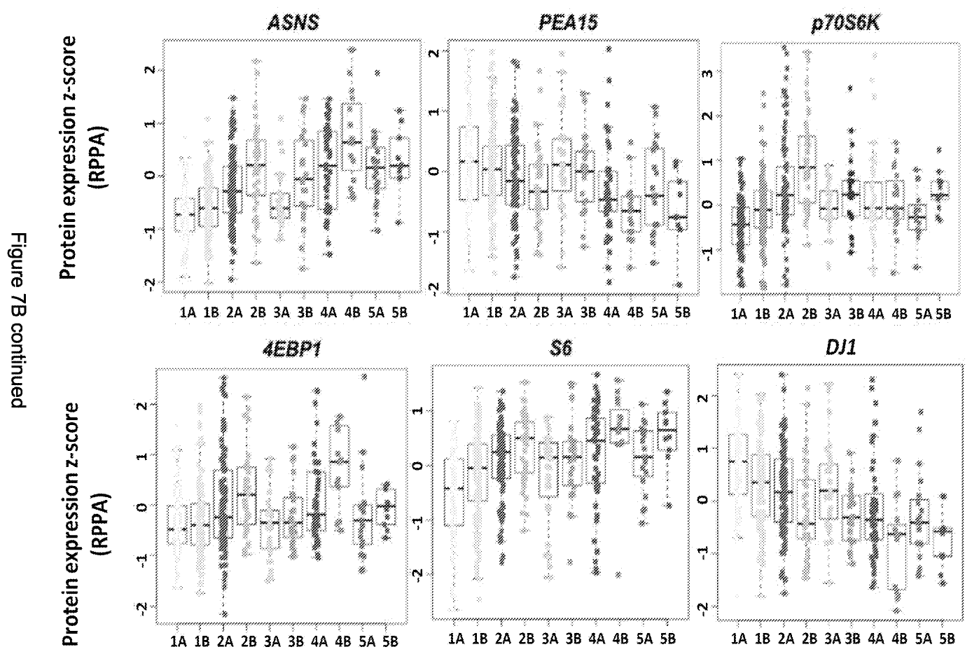

Tier-4 classification—the methods also include an additional tier of classification (tier-4) that involves classifying the above-identified subgroups from tier 1, tier 2 or tier 3 classification into additional subclasses based on each subgroup's median AG-score (genomic score). As illustrated in FIG. 7A, In early-stage ER-negative and TNBC high-risk subgroup cluster (identified from tier 3 classification, comprising Low_immune_score_Subgroups 2, 3, 4 and 5), this further classification step results in the identification of intermediate-risk cancer subgroups (those on the underside of each subgroup's median AG-score) along with high-risk subgroups (those on upper-side of each subgroup's median AG-score) that are characterised by relatively better and extremely poor prognosis, respectively. Similarly, this further classification step in the early-stage ER+ breast cancer subgroups identified intermediate-risk and high-risk cancer subgroups.

Since ER-negative breast cancers tend to have higher proliferation rates, the prognostic value of current multigene tests in these cancers is limited. Thus, there are no clinically helpful prognostic signatures for ER-negative cancers. Notably, the present molecular classification method leads to the stratification of ER-negative breast cancers into prognostic subgroups (FIG. 7A). Using the method described herein, it was possible identify around 40% of low-risk ER-negative breast cancer patients (with >90% 20-year overall survival probability and thus do not require any therapeutic intervention) and identify intermediate-risk patients.

As determined from various statistical approaches (such as GSEA, over-representation analysis and/or pathway/network enrichment analyses, etc.) and RPPA analyses, respectively, this effect on the prognosis is also reflected in each subgroups-specific biology and the degree of deregulation of the underlying biological/molecular mechanisms driving cancer growth in these respective subgroups (as illustrated in the examples provided from ER+ and ER-negative breast cancer cohorts in FIGS. 7B-7D showing RPPA signalling and mRNA expression of genes from various biological pathways). This information is highly useful for drug dose considerations, avoiding overtreatment and undertreatment through accurate patient stratification.

Notably, proteomic and gene expression data converge to similar biology driving each molecular group. Also, functional inference using protein data alone converged on biological networks highly similar to those obtained by transcriptome data.

Highly significantly, AG-subgroup analysis of primary and metastatic tumour specimens from the same breast cancer patients who developed metastatic breast cancer (The Metastatic Breast Cancer Project cancer dataset) revealed that in some breast cancer patients, the primary and metastatic tumours belong to distinct molecular subgroups (see Table 6). These findings are significant, as this provides an opportunity to develop a mechanistic understanding of the cancer evolution/progression from localised to the metastatic stage along with comprehensive information on the underlying disease (e.g. tumour) biology of primary and metastatic tumours to guide individualised clinical decision-making in metastatic late-stage breast cancer patients too.

Tier-5 classification—the methods also encompasses an additional fifth-tier of classification that involves classifying the above-identified subgroups from tier 1, tier 2, tier 3 or tier 4 classification steps into further subgroups based on the mutational profiles of genes (from Group A and Group B genomic aberrations list—Table 1) not already directly included in the tier 2 classification (apart from their broader contribution to AG-Score). This means, for example, in breast cancer, this would involve sub-classifying high-risk and low-risk groups into further subgroups based on, for instance, the tumour's CDH1 or MAP3K1 or GATA3 gene mutation statuses—the genomic aberrations from Group B not included in the tier 2 classification in breast cancer (being not a representative genomic aberration from each of Group A and Group B genomic aberrations). This additional fifth-tier classification helps determine the subgroup-specific role of various frequent gene mutations in cancer, independent of their overall contribution to AG-Score (for example, the role of CDH1 or MAP3K1 gene mutations in breast cancer AG-subgroups).

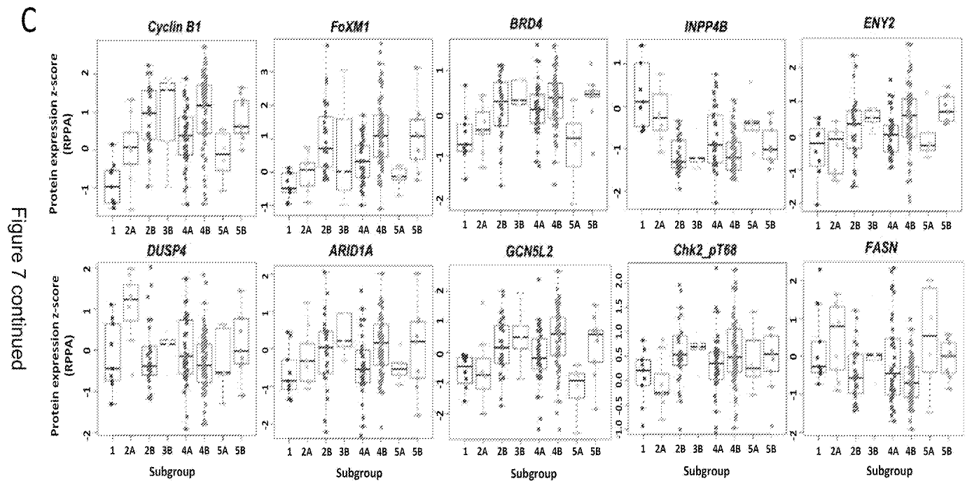

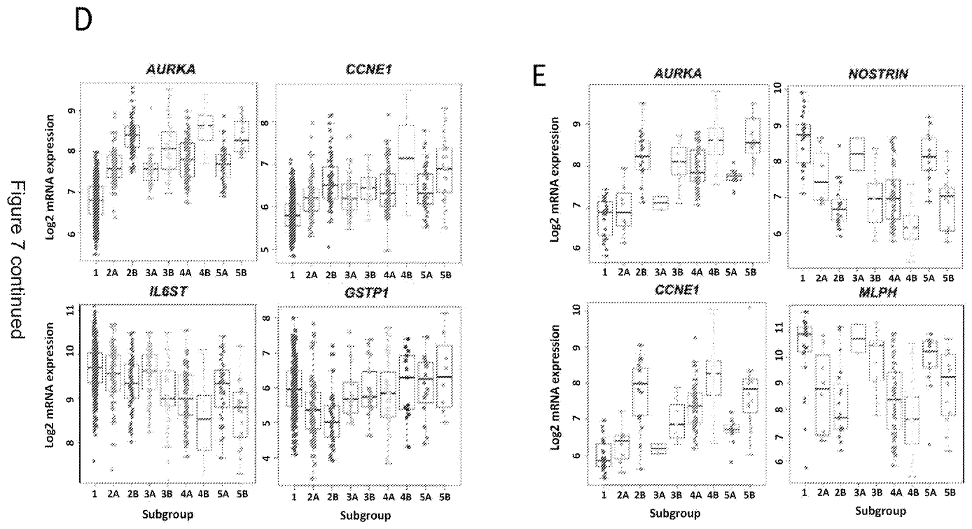

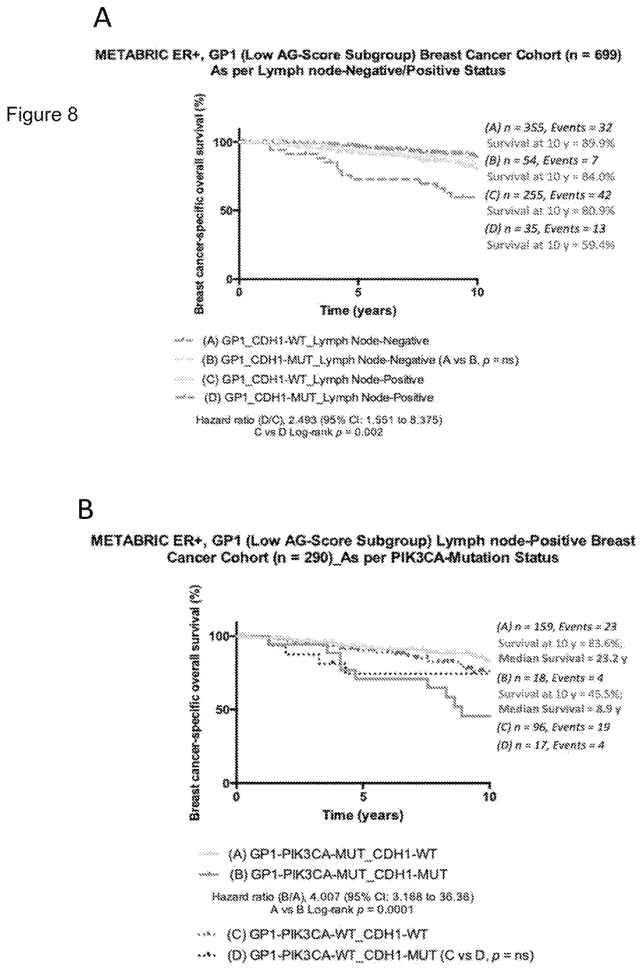

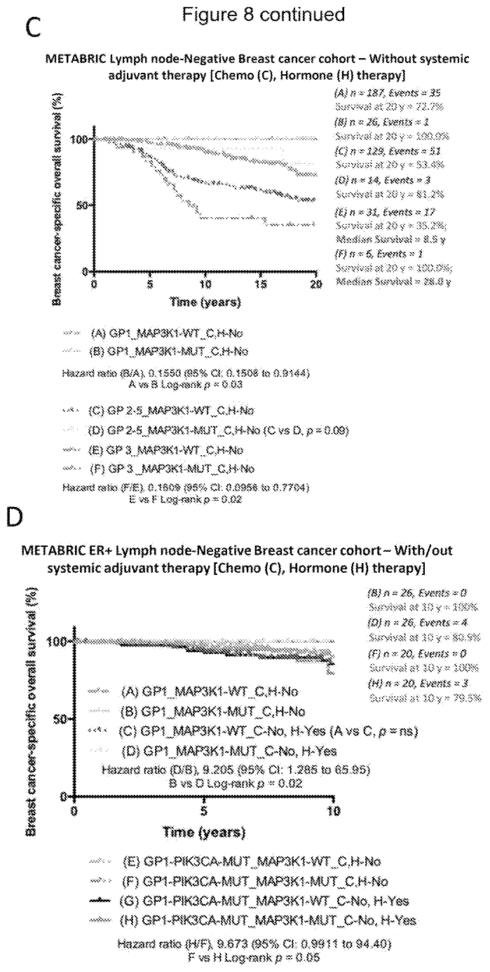

For example, previous studies (Ping et al., 2016) demonstrate that the CDH1 somatic mutation did not impact the prognosis of breast cancer patients with invasive histology and CDH1 mutations alone (with no classification applied) are not prognostic in breast cancer. However, as illustrated in FIGS. 8A and 8B, the additional classification step (tier-5) deciphers an AG-subgroup-specific negative prognostic role of CDH1 mutations in breast cancer. CDH1-mutants in Subgroup 1 lymph node-positive ER+ patients and Subgroup 2 and Subgroup 3 ER+ breast cancer patients (CDH1 mutations are enriched in these subgroups) had a negative prognostic role. Likewise, the subgroup-specific (from tier 1, tier 2, tier 3 or tier 4 classification steps) role of MAP3K1 (FIGS. 8C-8D) and GATA3. Notably, this additional classification step (tier-5) identified MAP3K1-mutant patient subsets from Subgroup 1 and Subgroup 3 (tier-2 high-risk subgroup) lymph node-negative ER+ breast cancer patient cohorts that could avoid overdiagnosis & overtreatment given their >90% 20-year survival probability even without systemic therapies (FIG. 8C). Also, the method identified MAP3K1-mutant patient subsets with relatively better prognoses from otherwise poor prognosis lymph node-positive (with >3 lymph node involvement) ER+ Subgroup 1 patients, benefiting from de-escalating chemotherapy and/or endocrine therapy regimens, avoiding overtreatment. FIG. 8D shows how hormone therapy treatment of MAP3K1-mutant patient subsets from Subgroup 1 lymph node-negative ER+ breast cancer patient cohorts could worsen (as a result of overdiagnosis and overtreatment) the overall survival of this MAP3K1-mutant patient subset with an excellent prognosis (given their >90% 20-year survival probability even without systemic therapies). This again highlights the AG-score independent roles of each Tier-5 subgroup, mainly attributed to their unique biologies.

This effect on the prognosis is also reflected in each subgroups-specific biology's and the degree of deregulation of the underlying biological/molecular mechanisms driving cancer growth in these respective subgroups (as illustrated from ER+ and ER-negative breast cancer cohorts in FIG. 8E showing RPPA signalling and mRNA expression of genes from various biological pathways. Also, tier-5 helped compare like with like, e.g., Subgroup 1-CDH1-MUT with Subgroup 1-CDH1-WT or Subgroup 1-MAP3K1-MUT with Subgroup 1-MAP3K1-WT. Since CDH1 mutations are primarily enriched in Subgroup 1, comparing all CDH1-mutants with CDH1-WT resulted in the enrichment of pathways mostly related to Subgroup 1 and not the pathways related to CDH1 mutations.

Incorporating the tier 5 classification step makes the present classification method an open-ended approach whereby any additional frequent genomic aberrations in cancer could be accommodated in the classification method, and their precise role in the cancer type could be determined (when genomic aberration frequencies are sufficient to perform a complete analysis).

Tier-6 classification—Proliferation pathways dominate the first and second-generation prognostic signatures. As mentioned above, shared sets of genes associated with Tier 1 disease-specific gene signatures may also be related to critical tumour growth-supporting pathways, for example, cell cycle and proliferation. However, tumour dissemination and metastasis are associated with cancer-progression-specific pathways, including invasion, EMT, metastasis, intravasation and dissemination. Notably, the low-risk (subgroup 1) Tier 1 group patients, despite having low enrichment of cell proliferation pathways, show a similar lymph node metastasis involvement to high-risk subgroup patients suggesting the involvement of independent factors contributing to metastasis and dissemination. Proliferation is a rate-limiting step of distant colonisation and is thus particularly important for assessing cancer prognosis if combined with additional information.

The method also encompasses an additional sixth-tier of classification that involves classifying the above-identified groups, subgroups and/or subsets from tier 1, tier 2, tier 3, tier4 and/or tier 5 (respectively) stratification steps into further subgroups (sub-risk groups) based on a metastatic risk score derived from a signature of dissemination. For example, for breast cancer, this would involve subclassifying groups, subgroups and/or subsets into further subgroups based on the metastatic risk score derived using the signature of dissemination.

The genes/proteins associated with metastasis and dissemination are identified in the backdrop of the dominant proliferation pathways by comparing primary tumours from non-metastatic tumours with the primary tumours from patients that develop metastatic tumours separately for each of the low and high-risk groups identified at Tier 1 level and/or any of tier 2, tier 3, tier 4 or tier 5 groups described above to identify DEGs and/or differentially expressed proteins within each of these two groups that would together constitute the signature of dissemination.

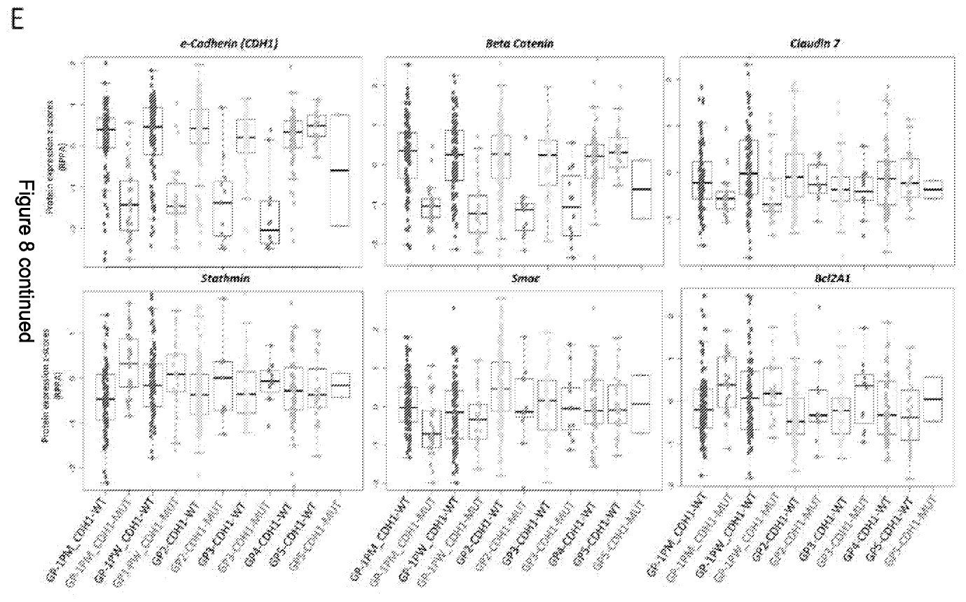

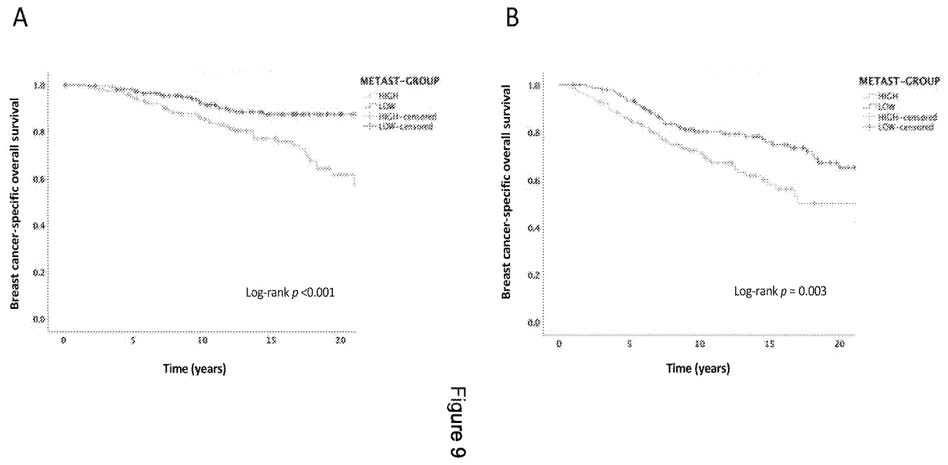

As an example, the primary tumours from lymph node-negative breast cancer patients who had no event 10 years after diagnosis were compared with the primary tumours from patients with three or more lymph node metastasis/involvement who had an event 10 years after diagnosis. This analysis was performed separately for each of the low and high-risk groups identified at the Tier 1 level to identify the genes associated with metastasis and dissemination in the backdrop of the dominant proliferation pathways constituting the signature of dissemination. The analysis together identified 271 unique DEGs with FDR<0.05 threshold. A similar analysis performed in the whole cohort together (without separate analyses for tier 1 low and high-risk groups) identifies striking 4260 DEGs (with FDR<0.05 threshold) dominated by cell proliferation pathways. Further application of univariate and multivariate Cox regression statistical approaches in ER+ HER2− breast cancer cohort led to the identification of the prognosis/survival-related DEGs to constitute a breast-specific metastatic gene signature (Table 8). As illustrated in FIGS. 9A and 9B, the additional classification step (tier-6) based on the metastatic risk score derived from the metastatic gene signature further stratifies the Tier 1 low and high-risk groups into further prognostic molecular subgroups. Importantly, the metastatic score and risk score derived at Tier 1 show a poor correlation (R²=0.138). The prognostic score derived from a signature of metastasis/dissemination from this further classification tier (tier 6) provides complementary and more personalised prognostic and predictive information for patients.

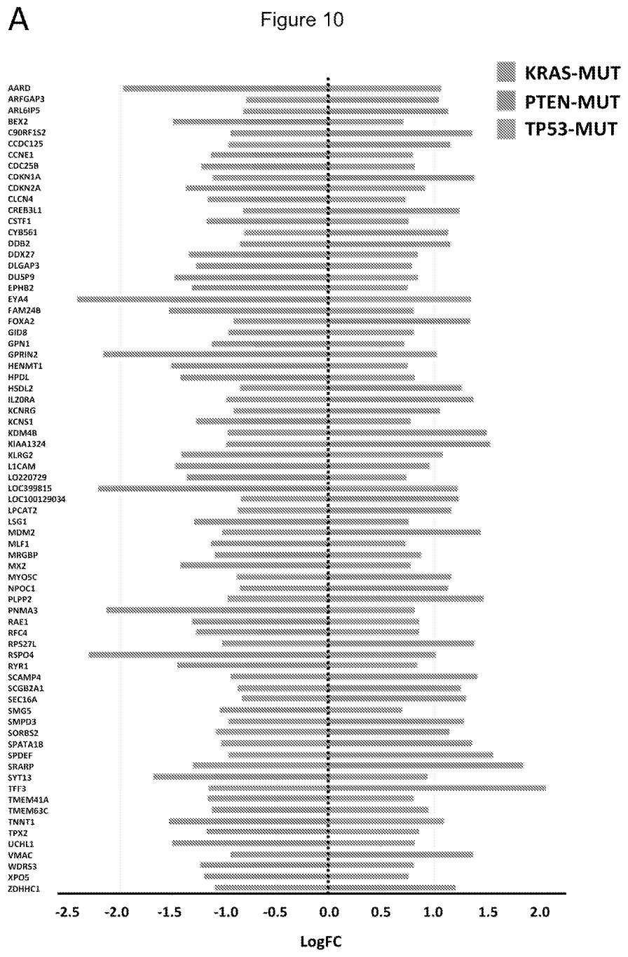

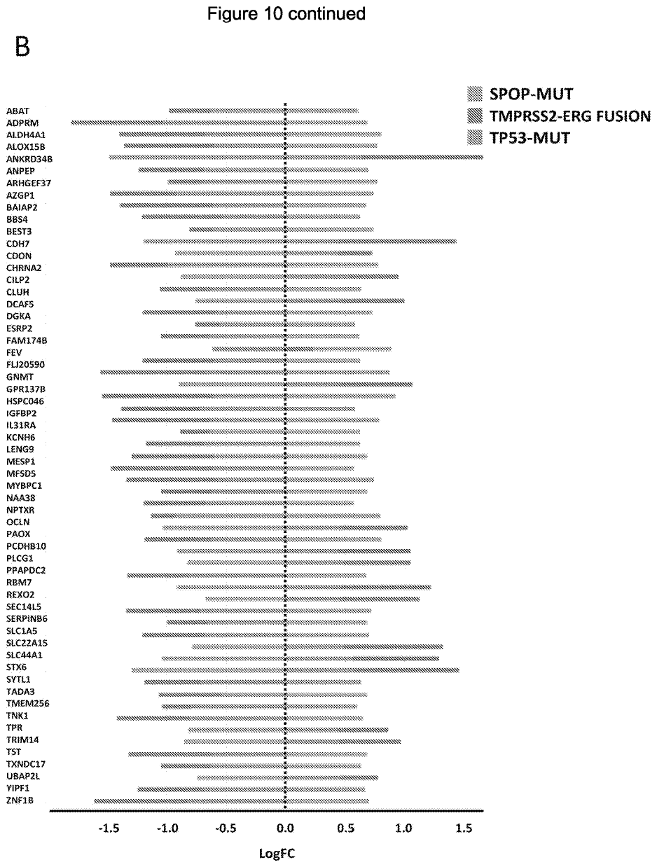

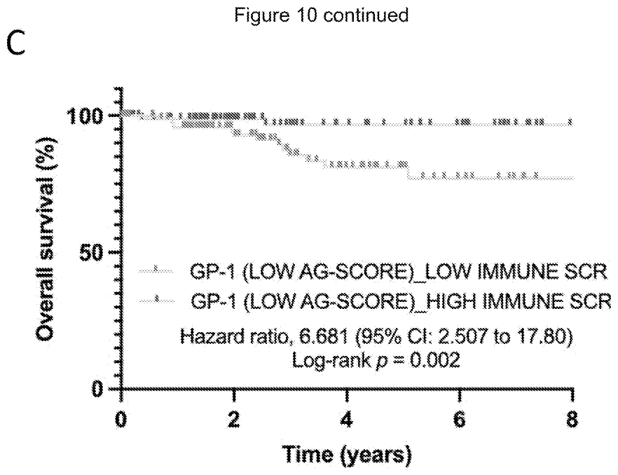

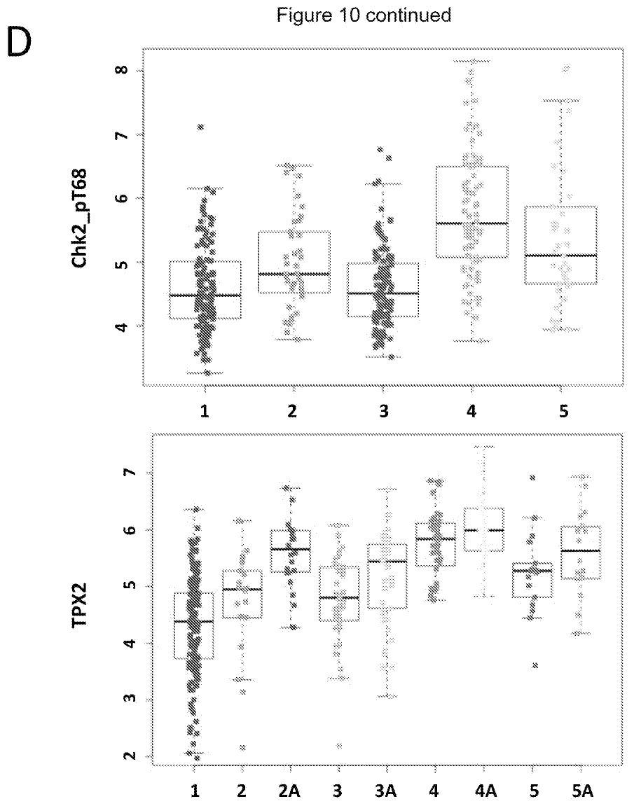

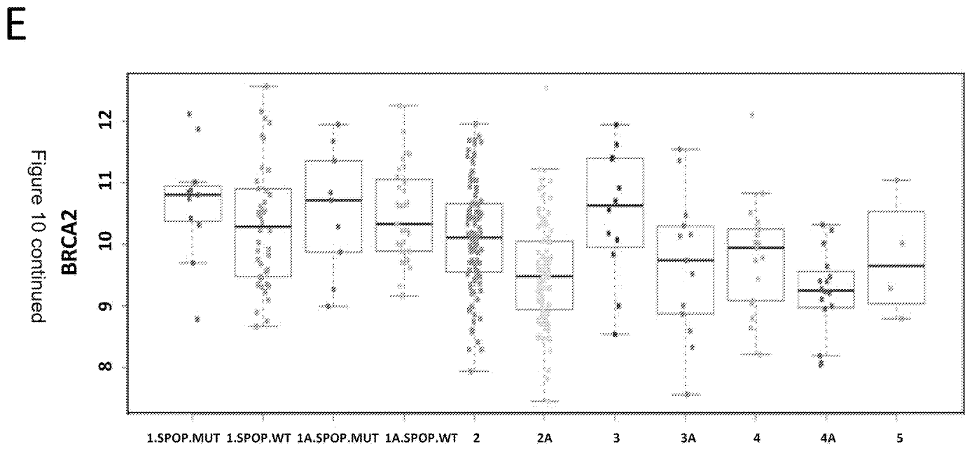

Example with other cancer types—The multi-tier classification method used herein applies to other diseases and other cancer types. Illustrated in FIGS. 10A-10E, are some examples showing how the multi-tier classification method used herein provides comprehensive information on the underlying tumour biology and accurate prognosis in endometrial and prostate cancer patients.

### CONCLUSION

Overall, analyses reveal that the multi-tier classification method used herein provides comprehensive information on the underlying tumour biology and accurate prognosis in cancer patients, independently of standard clinicopathological parameters. This integrated multi-tier analysis provides key molecular insights into tumour biology, which may directly affect treatment recommendations for patients. In addition, it provides opportunities for precision cancer medicine, biomarker-guided clinical trials and the development of novel drugs. Each identified subgroup is characterised by a unique set of biomarkers that could be used for guiding treatment decisions leading to improved quality of treatment and better therapy response for cancer patients. The methods help understand the nuance, context, biology and significance of the complex genomic alterations, thereby predicting the best clinical and therapeutic approach for cancer patients. The methods also provide accurate prognosis and risk assessment information for targeted cancer management and triaging by taking into careful account the genomic and immune landscape of each tumour. Although the discovery of molecular groups in this study was agnostic to patient outcomes, these groups were characterised by distinct and divergent patterns of clinical outcome (in terms of overall survival, relapse/disease-free survival, recurrence-free survival, metastasis-free survival, event-free survival, etc.). Notably, the association of molecular groups with outcomes was independent of molecular signatures of proliferation. The multi-tier classification method as described also represents a valuable tool to identify hormone receptor-positive BC patients at high risk (with poor clinical outcome) who might benefit from prolongation of endocrine therapy beyond the standard five years of treatment. This is an important question in the clinical management of ER+/HER2− BC patients who remain at persistent risk of recurrence for at least 15-20 years, with >50% of relapses and more than two-thirds of deaths occurring >5 years after the original diagnosis. However, while the continuation of endocrine therapy reduces the proclivity to develop late recurrences, its benefits must be weighed against side effects and quality of life, avoiding overtreatment through accurate patient stratification. Based on the analysis of sizeable patient cohorts, it is submitted that the classification method described here proves clinically valuable for the stratification of low-risk patients who might safely benefit from de-escalating endocrine therapy and/or chemotherapy regimens, thus avoiding overtreatment. On the other hand, this classification method could help identify high-risk patients who might benefit from more aggressive treatments. Additionally, the presented results identify a set of genes/proteins with a likely mechanistic role in the cancer progression process, which could represent novel molecular targets for the development of drugs counteracting the progression of cancer. The present method of tumour profiling is clinically valuable, either as a standalone test or in combination with clinicopathological parameters, to guide individualised clinical decision-making in cancer patients.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing examples. The invention extends to any novel one, or any novel combination, of the features disclosed in this specification (including any accompanying claims, abstract and drawings), or to any novel one, or any novel combination, of the steps of any method or process so disclosed.

### TABLES

TABLE 1
Breast Cancer-Specific List of Group
A And Group B Genomic Aberrations

| Group A | Group B | Not Determined |
|---|---|---|
| TP53 (34.4%) | PIK3CA (38.9%) | AHNAK (8.8%) |
| MUC16 (16.3%) | KMT2C (11.2%) | DNAH2 (7.4%) |
| TTN (16.4%)* | GATA3 (10.7%) | KMT2D (7.0%) |
| AHNAK2 (15.7%) | CDH1 (9.4%) | DNAH5 (6.7%) |
| SYNE1 (11.7%) | MAP3K1 (9.4%) | RYR2 (6.4%) |
| DNAH11 (9.0%) | MUC12 (5.5%)* | PDE4DIP (5.8%) |
| USH2A (6.8%) | MUC4 (5.4%)* | TG (5.5%) |
| HERC2 (9.4%) | CBFB (4.5%) | BIRC6 (5.1%) |
| AKAP9 (5.7%) | AKT1 (4.0%) | ERBB2 (2.8%) |
| FLG (4.6%)* | NCOR1 (4.1%)* | BRCA2 (1.8%) |
| PTEN (3.8%) | NF1 (3.7%) | |
| PIK3R1 (2.4%) | SF3B1 (2.6%) | |
| RB1 (2.2%) | FOXO3 (1.6%) | |
| BRCA1 (1.7%) | | |
| Most copy number alterations | | |
| Most gene fusions | | |

§ Gene mutation frequency (Percentage of samples with one or more mutations) in Metabric or TCGA Firehose Legacy Breast cancer datasets mentioned in parenthesis
*Mutation in the gene present only in Breast cancer TCGA Firehose Legacy dataset

TABLE 2
Prostate Cancer-Specific List of Group
A And Group B Genomic Aberrations

| GROUP A | GROUP B | NOT DETERMINED |
|---|---|---|
| TP53 (12.2%) | SPOP (11.4%) | FRG1BP (19.4%) |
| TTN (12.6%) | MUC17 (6.6%) | KMT2C (6.0%) |
| MUC16 (7.6%) | FOXA1 (5.6%) | |
| NBPF1 (5.4%) | | |
| SYNE1 (5.4%) | | |
| Most copy number alterations | | |
| Most gene fusions | | |

§ Gene mutation frequency (Percentage of samples with one or more mutations) in TCGA Firehose Legacy Prostate cancer dataset mentioned in parenthesis

TABLE 3
Endometrial Cancer-Specific List of Group
A And Group B Genomic Aberrations

| GROUP A | GROUP B |
|---|---|
| TP53 (21.9%) | PTEN (65.2%) |
| CHD4 (37.3%) | PIK3CA (50.1%) |
| FBXW7 (18.6%) | ARID1A (43.9%) |
| Most copy number alterations | KRAS (19.1%) |
| Most gene fusions | MUC16 (27.5%) |
| | TTN (39.8%) |
| | PIK3R1 (30.8%) |
| | KMT2D (27.7%) |
| | CTNNB1 (25.7%) |
| | RYR2 (24.2%) |
| | CTCF (24.6%) |
| | SYNE1 (22.1%) |
| | ATM (19.1%) |

§ Gene mutation frequency (Percentage of samples with one or more mutations) in TCGA PanCancer Atlas Uterine Corpus Endometrial Carcinoma mentioned in parenthesis

TABLE 4
Shared set of DEGs from top 200 statistically significant
(with FDR adjusted p-value < 0.05) DEGs between breast
cancer representative Group A and Group B genomic aberration's
(i.e., TP53 and PIK3CA gene mutations, respectively) gene
list in the METABRIC breast cancer dataset

| | Direction of | | Direction of |
|---|---|---|---|
| | association of | | association of |
| | RNA transcript | | RNA transcript |
| TP53_{—} | with TP53 | PIK3CA_{—} | with PIK3CA |
| METABRIC | gene mutation | METABRIC | gene mutation |
| AARS | 1 | AARS | −1 |
| ABAT | −1 | ABAT | 1 |
| ANLN | 1 | ANLN | −1 |
| ASPM | 1 | ASPM | −1 |
| AURKA | 1 | AURKA | −1 |
| AURKB | 1 | AURKB | −1 |
| BLM | 1 | BLM | −1 |
| BTD | −1 | BTD | 1 |
| BUB1 | 1 | BUB1 | −1 |
| C14orf45 | −1 | C14orf45 | 1 |
| CASC1 | −1 | CASC1 | 1 |
| CCNA2 | 1 | CCNA2 | −1 |
| CCNB2 | 1 | CCNB2 | −1 |
| CCNE1 | 1 | CCNE1 | −1 |
| CDC20 | 1 | CDC20 | −1 |
| CDC25B | 1 | CDC25B | −1 |
| CDC45 | 1 | CDC45 | −1 |
| CDCA3 | 1 | CDCA3 | −1 |
| CDCA5 | 1 | CDCA5 | −1 |
| CDCA8 | 1 | CDCA8 | −1 |
| CENPA | 1 | CENPA | −1 |
| CENPE | 1 | CENPE | −1 |
| CENPN | 1 | CENPN | −1 |
| CEP55 | 1 | CEP55 | −1 |
| CKAP2L | 1 | CKAP2L | −1 |
| CTPS | 1 | CTPS | −1 |
| CYBRD1 | −1 | CYBRD1 | 1 |
| DKC1 | 1 | DKC1 | −1 |
| E2F2 | 1 | E2F2 | −1 |
| EVL | −1 | EVL | 1 |
| FGD3 | −1 | FGD3 | 1 |
| FOXM1 | 1 | FOXM1 | −1 |
| GTPBP4 | 1 | GTPBP4 | −1 |
| HJURP | 1 | HJURP | −1 |
| KIF13B | −1 | KIF13B | 1 |
| KIF2C | 1 | KIF2C | −1 |
| KIFC1 | 1 | KIFC1 | −1 |
| LRP8 | 1 | LRP8 | −1 |
| MCM10 | 1 | MCM10 | −1 |
| MELK | 1 | MELK | −1 |
| NAT1 | −1 | NAT1 | 1 |
| NCAPG | 1 | NCAPG | −1 |
| NOSTRIN | −1 | NOSTRIN | 1 |
| ORC6 | 1 | ORC6 | −1 |
| PLK1 | 1 | PLK1 | −1 |
| POLQ | 1 | POLQ | −1 |
| PRR11 | 1 | PRR11 | −1 |
| PTTG1 | 1 | PTTG1 | −1 |
| PTTG3 | 1 | PTTG3 | −1 |
| SCUBE2 | −1 | SCUBE2 | 1 |
| SLC7A5 | 1 | SLC7A5 | −1 |
| STIL | 1 | STIL | −1 |
| STMN1 | 1 | STMN1 | −1 |
| TPX2 | 1 | TPX2 | −1 |
| TRIP13 | 1 | TRIP13 | −1 |
| TROAP | 1 | TROAP | −1 |
| UBE2C | 1 | UBE2C | −1 |

TABLE 5
List of Immune-Associated Genes for Deriving Immune Gene Signature And AG-Immune risk score calculations.

| | Direction of | | Direction of | | Direction of | | Direction of |
|---|---|---|---|---|---|---|---|
| | association of | | association of | | association of | | association of |
| | RNA transcript | | RNA transcript | | RNA transcript | | RNA transcript |
| | for AG- | | for AG- | | for AG- | | for AG- |
| Immune- | Immune | Immune- | Immune | Immune- | Immune | Immune- | Immune |
| Associated | risk score | Associated | risk score | Associated | risk score | Associated | risk score |
| Gene | calculations | Gene | calculations | Gene | calculations | Gene | calculations |
| GBP5 | 1 | LAG3 | 1 | CYTIP | 1 | MAL | 1 |
| CXCL9 | 1 | SLAMF1 | 1 | NCF1C | 1 | LCP1 | 1 |
| CCL5 | 1 | PLEK | 1 | FGL2 | 1 | UBASH3A | 1 |
| IDO1 | 1 | SP140 | 1 | P2RY8 | 1 | GZMH | 1 |
| GZMB | 1 | ARHGAP25 | 1 | SLA2 | 1 | ADAM7 | 1 |
| CD3D | 1 | ZBED2 | 1 | CD5 | 1 | RUNX3 | 1 |
| NFS1 | 1 | IL18RAP | 1 | IL7R | 1 | BU935198 | 1 |
| NKG7 | 1 | APOBEC3G | 1 | PSMB9 | 1 | PLAC8 | 1 |
| CD247 | 1 | CD27 | 1 | TNFSF13B | 1 | CXCR5 | 1 |
| CD7 | 1 | TBC1D10C | 1 | WAS | 1 | EVI2B | 1 |
| CTLA4 | 1 | LAMP3 | 1 | CD79A | 1 | BTN3A3 | 1 |
| CD2 | 1 | SPOCK2 | 1 | CASP1 | 1 | CD19 | 1 |
| GBP4 | 1 | LY9 | 1 | CD3E | 1 | IGLL1 | 1 |
| CD38 | 1 | STAT1 | 1 | FERMT3 | 1 | PSMB10 | 1 |
| ICOS | 1 | S1PR4 | 1 | ZAP70 | 1 | TNFRSF1B | 1 |
| GZMA | 1 | APOBEC3H | 1 | SELL | 1 | CAMK1G | 1 |
| GNLY | 1 | CXCL13 | 1 | LPXN | 1 | RASAL3 | 1 |
| IL18BP | 1 | HLA-F | 1 | GMFG | 1 | KLHL6 | 1 |
| CD8A | 1 | HLA-E | 1 | TNFAIP3 | 1 | SLAMF7 | 1 |
| TCRVB | 1 | GZMK | 1 | FCRH3 | 1 | MCOLN2 | 1 |
| CXCL10 | 1 | GIMAP5 | 1 | IRF8 | 1 | GPR18 | 1 |
| PTPRCAP | 1 | CORO1A | 1 | HCLS1 | 1 | C1QA | 1 |
| CXCR6 | 1 | CD53 | 1 | NCKAP1L | 1 | KLRD1 | 1 |
| SH2D1A | 1 | GIMAP4 | 1 | SASH3 | 1 | AMICA1 | 1 |
| CXCR3 | 1 | CCR7 | 1 | HLA-DMB | 1 | TNFRSF17 | 1 |
| PRF1 | 1 | WARS | 1 | RGL4 | 1 | CYBB | 1 |
| PVRIG | 1 | ARHGAP9 | 1 | CD79B | 1 | BTN3A1 | 1 |
| ITK | 1 | ZNF683 | 1 | BTLA | 1 | MYO1G | 1 |
| HCST | 1 | SAMD3 | 1 | ZNF831 | 1 | HLA-B | 1 |
| LTA | 1 | LTB | 1 | PSTPIP1 | 1 | IGFLR1 | 1 |
| PYHIN1 | 1 | PTPN7 | 1 | EPSTI1 | 1 | PIK3CD | 1 |
| CD3G | 1 | UBD | 1 | CD52 | 1 | ISG20 | 1 |
| GBP1 | 1 | CST7 | 1 | PDCD1 | 1 | INPP5D | 1 |
| PRKCB | 1 | RAC2 | 1 | ARHGAP15 | 1 | NCF4 | 1 |
| STAT4 | 1 | CTSW | 1 | TCRA | 1 | NLRC5 | 1 |
| IRF1 | 1 | PIM2 | 1 | CD86 | 1 | FCN1 | 1 |
| IFNG | 1 | SEMA4D | 1 | ARHGAP30 | 1 | TNFRSF4 | 1 |
| AIM2 | 1 | IL10RA | 1 | FGD2 | 1 | PPP1R16B | 1 |
| CD48 | 1 | HLA-DOB | 1 | CD72 | 1 | EOMES | 1 |
| IL21R | 1 | ADAMDEC1 | 1 | GIMAP7 | 1 | LCK | 1 |
| ITGB7 | 1 | ACAP1 | 1 | KLRB1 | 1 | CD69 | 1 |
| TAP1 | 1 | C5orf20 | 1 | TLR8 | 1 | NAPOR-1 | 1 |
| CD6 | 1 | TRAF3IP3 | 1 | C17orf87 | 1 | CCL8 | 1 |
| MAP4K1 | 1 | TRAT1 | 1 | CD97 | 1 | SLA | 1 |
| LILRB4 | 1 | IL2RG | 1 | POU2AF1 | 1 | LAIR2 | 1 |
| CD37 | 1 | SLC7A7 | 1 | KLRG1 | 1 | TRIM21 | 1 |
| ABI3 | 1 | abParts | 1 | VAV1 | 1 | SYK | 1 |
| MAFF | 1 | IL15 | 1 | KIAA1949 | 1 | C1orf54 | 1 |
| LRMP | 1 | MEI1 | 1 | SPI1 | 1 | XAF1 | 1 |
| HLA-DMA | 1 | LILRB3 | 1 | BTK | 1 | PTGDS | 1 |
| DOCK2 | 1 | GBP2 | 1 | DENND1C | 1 | FKBP11 | 1 |
| NUP62 | 1 | CCR2 | 1 | TCN2 | 1 | CCL2 | 1 |
| DKFZp686O16217 | 1 | LAT | 1 | LIME1 | 1 | LILRA5 | 1 |
| BATF2 | 1 | IL18R1 | 1 | GNGT2 | 1 | VAMP5 | 1 |
| CD40 | 1 | FCAR | 1 | RGS18 | 1 | NPL | 1 |
| RASSF5 | 1 | SEL1L3 | 1 | RCSD1 | 1 | CELF2 | 1 |
| BCL11B | 1 | TIGIT | 1 | CCL19 | 1 | RAB33A | 1 |
| PAG1 | 1 | TAGAP | 1 | APBB1IP | 1 | BG205162 | 1 |
| C1QB | 1 | HLA-DOA | 1 | STAMBPL1 | 1 | CMKLR1 | 1 |
| GVIN1 | 1 | TNFRSF13B | 1 | IFIH1 | 1 | FAM30A | 1 |
| CD4 | 1 | VCAM1 | 1 | PSME2 | 1 | HSD11B1 | 1 |
| TCRBV4S1A1T | 1 | APOBEC3F | 1 | TCRBV22S1A2N1T | 1 | EGFL6 | 1 |
| IL32 | 1 | PTPRC | 1 | CASP4 | 1 | OASL | 1 |
| CARD16 | 1 | FAM26F | 1 | LYN | 1 | RTP4 | 1 |
| PLCG2 | 1 | SRGN | 1 | DOK2 | 1 | TYMP | 1 |
| CCL4 | 1 | PLEKHO1 | 1 | ARRDC5 | 1 | MYO1F | 1 |
| IL2RA | 1 | FGR | 1 | HLA-DPB1 | 1 | RASGRP2 | 1 |
| ITGB2 | 1 | SAMSN1 | 1 | LY96 | 1 | CYBA | 1 |
| AKNA | 1 | NPSR1 | 1 | CSF2RA | 1 | WIPF1 | 1 |
| STX11 | 1 | SOCS1 | 1 | C2 | 1 | CCR1 | 1 |
| CCL13 | 1 | ETV7 | 1 | TNFSF14 | 1 | CLEC4A | 1 |
| C1orf162 | 1 | FLVCR2 | 1 | PARP14 | 1 | IL12RB1 | 1 |
| PATL2 | 1 | FPR3 | 1 | LOC643733 | 1 | LGALS2 | 1 |
| HLA-DQA1 | 1 | PNOC | 1 | WNT10A | 1 | IGJ | 1 |
| PARVG | 1 | NCR3 | 1 | CD83 | 1 | SLC2A6 | 1 |
| FASLG | 1 | TRIM69 | 1 | ARHGAP4 | 1 | CD84 | 1 |
| NCF1 | 1 | THEMIS | 1 | SPIB | 1 | PRDM1 | 1 |
| CTSC | 1 | RASSF4 | 1 | NAPSB | 1 | IFI16 | 1 |
| GPSM3 | 1 | GPR183 | 1 | VPREB3 | 1 | CD8B | 1 |
| TRPV2 | 1 | GIMAP6 | 1 | LRRC33 | 1 | DEF6 | 1 |
| P2RY10 | 1 | BTN3A2 | 1 | IL15RA | 1 | ZBTB32 | 1 |
| TRAF1 | 1 | IGSF6 | 1 | DOCK10 | 1 | GPR65 | 1 |
| LCP2 | 1 | LAPTM5 | 1 | BANK1 | 1 | POP5 | −1 |
| IGLL3P | 1 | HLA-DPA1 | 1 | PTPN22 | 1 | TLR10 | 1 |
| HLA-G | 1 | HLA-DRA | 1 | LAP3 | 1 | FCRL3 | 1 |
| CLECL1 | 1 | MZB1 | 1 | SLC15A3 | 1 | CD40LG | 1 |
| IFI30 | 1 | DOCK8 | 1 | TCL1A | 1 | LST1 | 1 |
| TMEM140 | 1 | PLCL2 | 1 | RASSF2 | 1 | FYN | 1 |
| GPR132 | 1 | TNFRSF9 | 1 | SLC9A9 | 1 | RNF126P1 | 1 |
| TIFAB | 1 | C1QC | 1 | STK4 | 1 | CD1B | 1 |
| PTAFR | 1 | MNDA | 1 | PITPNM2 | 1 | GAB3 | 1 |
| CTSH | 1 | EFHD2 | 1 | FAM46C | 1 | PLCB2 | 1 |
| CD163 | 1 | RGS19 | 1 | ST8SIA4 | 1 | FCER1G | 1 |
| TYROBP | 1 | KLRC1 | 1 | LAT2 | 1 | AA215505 | 1 |
| AIF1 | 1 | PLA1A | 1 | ALOX5 | 1 | RNASE6 | 1 |
| TEX11 | 1 | IFIT3 | 1 | CLEC12A | 1 | FBXO6 | 1 |
| LRRC8C | 1 | NAPSA | 1 | PTPN6 | 1 | MX2 | 1 |
| B2M | 1 | CSF1R | 1 | KIAA0748 | 1 | RAB8B | 1 |
| MLKL | 1 | FCRL5 | 1 | CD74 | 1 | PSMB8 | 1 |
| HLA-H | 1 | PIM1 | 1 | PTGDR | 1 | DKFZp666C094 | 1 |
| APOBEC3C | 1 | TAPBPL | 1 | PLEKHO2 | 1 | LYSMD2 | 1 |
| IRF4 | 1 | P2RX1 | 1 | GIMAP2 | 1 | EVI2A | 1 |

TABLE 6
AG-Group stratification in Metastatic Breast Cancer cohort

| | Pathological | AG- | AG- |
|---|---|---|---|
| | Procedure | GROUP I | GROUP |
| Patient ID | Location | (Tier 2) | (Tier 4) |
| MBCProject_4MF1FIFQ | BREAST | 4 | 4A |
| MBCProject_4MF1FIFQ | BREAST | 4 | 4B |
| MBCProject_5gHasou8 | BREAST | 2 | 2A |
| MBCProject_5gHasou8 | BREAST | 2 | 2B |
| MBCProject_6vTVHzur | BREAST | 5 | 5B |
| MBCProject_6vTVHzur | CHEST | 3 | 3B |
| | WALL | | |
| MBCProject_7JtRIPI2 | BREAST | 2 | 2A |
| MBCProject_7JtRIPI2 | BREAST | 1 | 1_PIK3CA- |
| | | | TP53-WT |
| MBCProject_8bfWIQu1 | BREAST | 4 | 4B |
| MBCProject_8bfWIQu1 | BRAIN | 4 | 4B |
| MBCProject_B7f5hRfD | BREAST | 2 | 2A |
| MBCProject_B7f5hRfD | BREAST | 2 | 2B |
| MBCProject_BOHWtZT8 | LIVER | 4 | 4B |
| MBCProject_BOHWtZT8 | BREAST | 5 | 5B |
| MBCProject_d5CbUNTbIl | CHEST | 4 | 4B |
| | WALL | | |
| MBCProject_d5CbUNTb | BREAST | 4 | 4B |
| MBCProject_eXSVsxUm | BREAST | 2 | 2B |
| MBCProject_eXSVsxUm | BREAST | 1 | 1 PIK3CA- |
| | | | TP53-WT |
| MBCProject_gASyuQSI | BREAST | 1 | 1_PIK3CA- |
| | | | TP53-WT |
| MBCProject_gASyuQSI | AXILLARY | 1 | 1_PIK3CA- |
| | LYMPH | | TP53-WT |
| | NODE | | |
| MBCProject_gASyuQSI | BREAST | 1 | 1_PIK3CA- |
| | | | TP53-WT |
| MBCProject_gotjfgf0 | BREAST | 4 | 4B |
| MBCProject_gotjfgf0 | BONE | 4 | 4A |
| MBCProject_JGcLUJiA | AXILLARY | 3 | 3B |
| | LYMPH | | |
| | NODE | | |
| MBCProject_JGcLUJiA | PLEURAL | 3 | 3B |
| | FLUID | | |
| MBCProject_JGcLUJiA | BREAST | 3 | 3B |
| MBCProject_KGs1fxH2 | BREAST | 1 | 1_PIK3CA- |
| | | | TP53-WT |
| MBCProject_KGs1fxH2 | BREAST | 3 | 3A |
| MBCProject_mrIQIdsx | PLEURA | 2 | 2B |
| MBCProject_mrIQIdsx | BRAIN- | 5 | 5A |
| | CNS | | |
| MBCProject_N4srsKsr | BREAST | 2 | 2A |
| MBCProject_N4srsKsr | BREAST | 2 | 2A |
| MBCProject_nkU3hDhB | BREAST | 1 | 1_PIK3CA- |
| | | | MUT, |
| | | | TP53-WT |
| MBCProject_nkU3hDhB | BREAST | 1 | 1_PIK3CA- |
| | | | MUT, |
| | | | TP53-WT |
| MBCProject_nkU3hDhB | BREAST | 3 | 3A |
| MBCProject_oqupfDu7 | AXILLARY | 5 | 5A |
| | LYMPH | | |
| | NODE | | |
| MBCProject_oqupfDu7 | BREAST | 5 | 5B |
| MBCProject_pktAIxFb | BREAST | 2 | 2B |
| MBCProject_pktAIxFb | BREAST | 2 | 2B |
| MBCProject_PKTDsOSa | BREAST | 4 | 4A |
| MBCProject_PkTDsOSa | BREAST | 4 | 4A |
| MBCProject_QbiZiytx | BREAST | 1 | 1_PIK3CA- |
| | | | TP53-WT |
| MBCProject_QbiZiytx | BREAST | 2 | 2A |
| MBCProject_qmu6TYto | BREAST | 1 | 1_PIK3CA- |
| | | | MUT, |
| | | | TP53-WT |
| MBCProject_qmu6TYto | BREAST | 1 | 1_PIK3CA- |
| | | | TP53-WT |
| MBCProject_W4FBsLSx | AXILLARY | 2 | 2B |
| | LYMPH | | |
| | NODE | | |
| MBCProject_W4FBsLSx | AXILLARY | 4 | 4B |
| | LYMPH | | |
| | NODE | | |
| MBCProject_W4FBsLSx | CERVICAL | 4 | 4B |
| | LYMPH | | |
| | NODE | | |
| MBCProject_WYHpfXf1 | BREAST | 5 | 5B |
| MBCProject_WYHpfXf1 | BREAST | 5 | 5A |
| MBCProject_xBfJfri9 | BREAST | 1 | 1_PIK3CA- |
| | | | TP53-WT |
| MBCProject_xBfJfri9 | BREAST | 1 | 1_PIK3CA- |
| | | | TP53-WT |
| MBCProject_Y7iLIOU0 | BREAST | 2 | 2A |
| MBCProject_Y7iLIOU0 | BREAST | 2 | 2A |
| MBCProject_ygcMFgCR | BREAST | 2 | 2B |
| MBCProject_ygcMFgCR | BREAST | 2 | 2B |
| MBCProject_ygcMFgCR | BREAST | 2 | 2B |
| MBCProject_zyt5TKFB | BREAST | 2 | 2B |
| MBCProject_zyt5TKFB | BREAST | 1 | 1_PIK3CA- |
| | | | MUT, |
| | | | TP53-WT |

TABLE 7
List of Breast cancer-specific prognostic genes selected from the common Top 3500 statistically significant
shared DEG list (FDR < 0.05) between the representative Group A and Group B genomic aberrations from
Metabric and TCGA breast cancer datasets after the multivariate Cox analysis (p < 0.001 cut-off) in
ER+ HER2− Metabric breast cancer patient cohort (clinical factors considered for multivariate cox
analysis include tumour stage, tumour size, histological grade, inferred menopausal status, age, lymph nodal
status, cellularity, Nottingham prognostic index, chemotherapy, hormone and radiation therapy treatment status).
Coefficient (β), the regression coefficient of the multivariate Cox analysis, is provided for each target gene.

| Prognosis | | Prognosis | | Prognosis | | Prognosis | |
|---|---|---|---|---|---|---|---|
| related | coefficient | related | coefficient | related | coefficient | related | coefficient |
| DEGs | (β) | DEGs | (β) | DEGs | (β) | DEGs | (β) |
| KIF20A | 0.46246231 | MKI67 | 0.33033168 | PRC1 | 0.28507721 | FBXW4 | −0.2360679 |
| FOXM1 | 0.45361116 | NUSAP1 | 0.3232409 | KIFC1 | 0.30176996 | FANCD2 | 0.25765997 |
| PTTG1 | 0.42859331 | CENPF | 0.33161175 | ATAD2 | 0.2533792 | PKMYT1 | 0.2428945 |
| CKAP2L | 0.4342007 | PHF19 | 0.31587377 | DNAJB11 | 0.28253566 | PARP3 | −0.2386177 |
| CDC20 | 0.45129689 | CENPA | 0.3756038 | CDC25C | 0.24693399 | UCK2 | 0.26658575 |
| CDCA5 | 0.40658632 | TPX2 | 0.34132599 | NUP93 | 0.29301481 | SUSD3 | −0.2432105 |
| TROAP | 0.37735579 | SHMT2 | 0.28668389 | HSD17B8 | −0.2714294 | LYPD6 | −0.2295771 |
| BIRC5 | 0.41364054 | LRIG1 | −0.3149489 | FBXO5 | 0.22750293 | NCAPD2 | 0.27732936 |
| GTSE1 | 0.39668175 | KIF2C | 0.34671451 | STIP1 | 0.2467202 | UBE2T | 0.24162196 |
| MELK | 0.41973861 | TOP2A | 0.32549568 | MCM2 | 0.27139867 | NOSTRIN | −0.2642631 |
| BUB1 | 0.41866589 | MCM10 | 0.34508796 | MCM6 | 0.26890172 | CDC25A | 0.274341 |
| RACGAP1 | 0.36582736 | RAD51AP1 | 0.32299679 | CYB5D2 | −0.2737303 | GINS3 | 0.22145615 |
| CCNA2 | 0.38951372 | CEP55 | 0.33322782 | NUDT18 | −0.247061 | ERCC6L | 0.23900456 |
| EXO1 | 0.38495087 | CDCA3 | 0.33852625 | DYNLRB2 | −0.2651523 | LMNB2 | 0.25306406 |
| UBE2C | 0.37666675 | AURKB | 0.3360582 | RAD54L | 0.27236433 | PEX11G | −0.2240988 |
| YEATS2 | 0.32243189 | CDCA8 | 0.33498183 | CCNB1 | 0.24896977 | KIF15 | 0.22922756 |
| ASPM | 0.37238301 | KIF23 | 0.31684212 | GMPS | 0.30055145 | KIF18A | 0.23644056 |
| SKP2 | 0.38569072 | PUF60 | 0.26375619 | STMN1 | 0.25864192 | SPC25 | 0.22394426 |
| FAM83D | 0.34747497 | TTK | 0.31660139 | UBE2S | 0.25178003 | OIP5 | 0.22293051 |
| KIF14 | 0.36256864 | CENPN | 0.3014093 | CYB561D2 | −0.2520021 | IQUB | −0.2292705 |
| FEN1 | 0.30864372 | E2F2 | 0.31866497 | MAPT | −0.2733905 | ESPL1 | 0.22320694 |
| POLQ | 0.355164 | E2F7 | 0.24348129 | STIL | 0.27524046 | SLC25A19 | 0.2315413 |
| AURKA | 0.32365295 | CBX2 | 0.29693482 | TRIP13 | 0.27894201 | LRP8 | 0.26459075 |
| CCNB2 | 0.35764275 | CENPE | 0.30193934 | RUNDC1 | −0.2716138 | E2F3 | 0.23941965 |
| PLK1 | 0.35483838 | KIF4A | 0.29419823 | CDCA2 | 0.3180007 | XPOT | 0.22700369 |
| PLK4 | 0.31119027 | PHYHD1 | −0.2752146 | BOP1 | 0.22987058 | TYMS | 0.22732865 |
| CDCA7 | 0.36382966 | GPR19 | 0.22502056 | FGD3 | −0.2397713 | | |

TABLE 8
List of Breast cancer-specific prognostic metastatic/dissemination associated genes selected
after the multivariate Cox analysis (p < 0.01 cut-off) of DEGs identified in Metabric
ER+ HER2− breast cancer cohort analysis where the primary tumours from lymph
node-negative breast cancer patients who had no event 10 years after diagnosis were compared
with the primary tumours from patients with three or more lymph node metastasis/involvement
who had an event 10 years after diagnosis separately performed in each of the low and
high-risk groups identified at the Tier 1 level (clinical factors considered for multivariate
cox analysis include tumour stage, tumour size, histological grade, inferred menopausal
status, age, lymph nodal status, cellularity, Nottingham prognostic index, chemotherapy,
hormone and radiation therapy treatment status). Coefficient (β), the regression coefficient
of the multivariate Cox analysis, is provided for each target gene.

| Metastasis | | Metastasis | | Metastasis | | Metastasis | |
|---|---|---|---|---|---|---|---|
| related | coefficient | related | coefficient | related | coefficient | related | coefficient |
| DEGs | (β) | DEGs | (β) | DEGs | (β) | DEGs | (β) |
| SEMA3B | −0.222 | DIRAS3 | −0.315 | CHDH | −0.119 | LCN12 | −0.165 |
| DRC3 | −0.275 | RGS22 | −0.203 | AGO4 | −0.201 | DCAKD | −0.166 |
| SPATA4 | −0.247 | CR593862 | −0.222 | CPT1A | 0.157 | DBNDD2 | −0.195 |
| GREM1 | 0.227 | ENC1 | 0.288 | CSPG4 | 0.186 | TMEM101 | −0.166 |
| SLC7A2 | −0.22 | TMEM26 | −0.209 | NBPF22P | −0.182 | CAPN8 | −0.189 |
| CELSR2 | −0.274 | MYL5 | −0.221 | SLC15A2 | 0.215 | GATAD1 | −0.174 |
| MCU | 0.252 | RGS4 | 0.207 | RPGR | −0.19 | CASP3 | 0.176 |
| FLNB | −0.243 | EIF2B2 | 0.186 | DDIT4 | 0.183 | AAR2 | 0.157 |
| TBC1D31 | 0.261 | NXNL2 | −0.205 | MYCBPAP | −0.17 | F8 | −0.177 |

### CLAUSES

1. A method of classifying one or more genomic aberrations associated with a disease, the method comprising:
a. identifying genes in a plurality of control subjects suffering from the disease that undergo a change of expression in response to a first genomic aberration and selecting the genes that undergo a change of expression in response to the first genomic aberration to provide a first set of differentially expressed genes (DEGs) associated with the first genomic aberration;
b. identifying DEGs of the first set of DEGs that overlap with DEGs of a control set of DEGs for a control genomic aberration and selecting the overlapping DEGs to form a first set of overlapping DEGs;
c. comparing the fold direction of change of expression of each DEG of the first set of overlapping DEGs to a fold direction change of expression of the corresponding DEG of the control set of DEGs;
d. classifying the first genomic aberration into a first or second group wherein:
   i. the first group comprises at least 51% overlapping DEGs that comprises a fold direction of change of expression that is the same as the fold direction of change of expression as the corresponding DEG of the control genomic aberration (Group A);
   ii. the second group comprises at least 51% overlapping DEGs that comprise a fold direction of change of expression that is inverse to the fold direction of change of expression of the corresponding DEG of the control genomic aberration (Group B).

2. The method of clause 1, wherein the method comprises stratifying a subject suffering from the disease, wherein stratifying comprises;
calculating a risk score for the subject based on the classified genomic aberration and
the subject is stratified based on the risk score; and wherein the calculated risk score is indicative of prognosis of the subject.

3. The method of clause 1 or 2, wherein the method further comprises:
a. classifying at least one genomic aberration associated with the disease as Group A and at least one second genomic aberration associated with the disease as Group B;
b. selecting a representative Group A genomic aberration and a representative Group B genomic aberration;
   wherein each representative genomic aberration is selected based on the frequency of occurrence of the genomic aberration for disease and the number of DEGs associated with the genomic aberration.

4. The method of any of clauses 1 to 3, wherein the method further comprises classifying one or more further genomic aberrations associated with the disease by comparing DEGs associated with the one or more further genomic aberrations to the same DEGs of the first set of overlapping DEGs of the representative Group A genomic aberration and the representative Group B genomic aberration and determining a similarity between the fold direction change of the DEGS and classifying the one or more further genomic aberrations as a Group A or Group B genomic aberration based on the similarity.

5. The method of clauses 3 or 4, wherein the method further comprises:
a. determining DEGs associated with the representative Group A genomic aberration and/or one or more further Group A genomic aberrations that overlap with DEGs associated with the representative Group B genomic aberration and/or one or more further Group B genomic aberrations and selecting the overlapping DEGs to form a shared set of DEGs;
b. selecting two or more DEGs of the shared set of DEGs to provide one or more disease-specific gene signatures wherein each disease-specific gene signature comprises at least one DEG that has a direction of fold change of expression that is inverse between the representative Group A and representative Group B genomic aberrations;
c. providing an expression level for each DEG of each disease-specific gene signature based on a level of RNA transcript for each DEG for the plurality of control subjects;
d. providing an expression level of each DEG of the disease-specific gene signature for the subject;
e. calculating the risk score for the subject based on the fold direction change of expression and/or the expression level of each DEG of each disease-specific gene signature for the plurality of control subjects;
   wherein the subject is stratified as high risk or a low risk based on the calculated risk score (Tier 1 groups).

6. The method of clause 5, wherein calculating the risk score comprises:
a. sorting the expression level of each DEG of the disease-specific gene signature of the plurality of control subjects that is the upregulated for the corresponding DEG of the representative Group A genomic aberration, one or more further Group A genomic aberrations and/or control genomic aberration in ascending order of the expression level; and
   dividing the sorted expression levels into 1 to n fractions based on a dynamic range of the expression levels for the plurality of control subjects; and
   assigning a relative expression value for each fraction, wherein the relative expression value is 1 to n for each corresponding fraction from lowest to highest expression level;
b. sorting the expression level of each DEG of the at least one disease-specific gene signature of the plurality of control subjects that downregulated for the corresponding DEG of the representative Group A genomic aberration, one or more further Group A genomic aberrations and/or control genomic aberration in ascending order of the expression level; and
   dividing the sorted expression levels into 1 to n fractions based on a dynamic range of the expression levels for the plurality of control subjects; and
   assigning a relative expression value for each fraction, wherein the relative expression value is 1 to n for each corresponding fraction from highest to lowest expression level;
c. comparing the expression level of each DEG of the disease-specific gene signature of the subject to the expression levels of each DEG of the disease-specific gene signature of the plurality of control subjects;
d. assigning a relative expression value to each DEG of the disease-specific gene signature of the subject based on the expression value assigned to the corresponding DEG of the disease-specific gene signature of the plurality of control subjects in steps a and b;
e. calculating the sum of the relative expression values assigned to each DEG of the disease-specific gene signature of the subject to provide the risk score.

7. The method of clause 5, wherein calculating the risk score comprises:
calculating the difference between:
   the sum of the expression level of each DEG of the disease-specific gene signature of the subject that upregulated for the corresponding DEG of the representative Group A genomic aberration, one or more further Group A genomic aberrations and/or control; and
   the sum of the expression level of each DEG of the disease-specific gene signature of the subject that is downregulated for the corresponding DEG of the representative Group A genomic aberration, one or more further Group A genomic aberrations and/or control genomic aberration.

8. The method of clause 5, wherein calculating the risk score comprises:
calculating a ratio of expression levels for each DEG of the disease-specific gene signature of the subject that is upregulated the corresponding DEG of the representative Group A genomic aberration, one or more further Group A genomic aberrations and/or control genomic aberration to;
the expression levels for each DEG of the disease-specific gene signature of the subject that is downregulated for the corresponding DEG of the representative Group A genomic aberration, one or more further Group A genomic aberrations and/or control genomic aberration.

9. The method of any of clauses 4 to 8, wherein selecting two or more DEGs of the shared set of DEGs to provide the one or more disease-specific gene signatures comprises selecting at least two DEGs from the shared set of DEGs having a statistical significance lower than a threshold value.

10. The method of any preceding clause, wherein the control genomic aberration is selected from the most frequently occurring genomic aberrations for the disease.

11. The method of any preceding clause, wherein the disease is cancer.

12. The method of clause 11, wherein the control genomic aberration comprises at least one TP53 gene mutation.

13. The method of clause 11 or 12, wherein:
a. the cancer is breast cancer and
   i. the representative Group A genomic aberration comprises TP53 gene mutations; and
   ii. the representative Group B genomic aberration comprises PIK3CA gene mutations;
b. the cancer is prostate cancer and
   i. the representative Group A genomic aberration comprises TP53 gene mutations; and
   ii. the representative Group B genomic aberration comprises SPOP gene mutations; or
c. the cancer is endometrial cancer and
   i. the representative Group A genomic aberration comprises TP53 gene mutations; and
   ii. the representative Group B genomic aberration comprises PTEN gene mutations.

14. The method of any of clauses 1 to 13 wherein the method further comprises stratifying the subject into one or more risk subgroups (Tier 2 groups) based on the mutational status of the representative Group A and/or Group B genomic aberrations;
wherein the one or more risk subgroups (Tier 2 groups) comprise a further indicator of prognosis.

15. The method of clause 14, wherein:
a. the disease is breast cancer and wherein the risk subgroup is selected from:
   i. TP53-WT and PIK3CA-WT;
   ii. TP53-WT and PIK3CA-MUT;
   iii. TP53-MUT and PIK3CA-WT; or
   iv. TP53-MUT and PIK3CA-MUT;
b. the disease is prostate cancer and wherein the risk subgroup is selected from:
   i. TP53-WT and SPOP-WT;
   ii. TP53-WT and SPOP-MUT;
   iii. TP53-MUT and SPOP-WT; or
   iv. TP53-MUT and SPOP-MUT; or
c. the disease is endometrial cancer and wherein the risk subgroup is selected from:
   i. TP53-WT and PTEN-WT;
   ii. TP53-WT and PTEN-MUT;
   iii. TP53-MUT and PTEN-WT; or
   iv. TP53-MUT and PTEN-MUT.

16. The method of clause 14 or 15, wherein the one or more risk subgroups (Tier 2 groups) comprises mutational status of one or more further Group A and/or Group B genomic aberrations.

17. A method of calculating an immune score for a subject suffering from a disease, the disease comprising one or more genomic aberrations, the method comprising:
a. selecting two or more immune associated genes associated with disease to form at least one immune signature;
b. assigning a direction of association to each gene of the immune signature based on a change of expression for a plurality of control subjects;
   wherein each gene with an increased expression level is designated with a direction of association and each gene with a decreased expression is designated with a second direction of association inverse to the first direction of association;
c. determining an expression level for each gene of the immune signature based on a level of RNA transcript for each gene for the plurality of control subjects; and
d. determining an expression level for each gene of the immune signature based on a level of RNA transcript for each gene for the subject.

18. The method of clause 17, wherein the method further comprises stratifying the subject wherein stratifying comprises,
calculating an immune score based on the direction of association and the expression level of the each gene of immune signature of the subject
stratifying the subject as high immune risk or a low immune risk based on the calculated immune score; and
   wherein the immune score is indicative of a prognosis of the subject.

19. The method of clause 18, wherein calculating the immune score comprises:
a. sorting the expression level of each immune associated gene of the immune signature for the plurality of control subjects having the first direction of association of expression in ascending order of the expression level; and
   dividing the sorted expression levels into 1 to n fractions based on a dynamic range of the expression levels for the plurality of control subjects; and
   assigning a relative expression value for each fraction, wherein the relative expression value is 1 to n for each corresponding fraction from lowest to highest expression level;
b. sorting the expression level of each immune associated gene of the immune signature for the plurality of control subjects having the second direction of association in ascending order of the expression level; and
   dividing the sorted expression levels into 1 to n fractions based on a dynamic range of the expression levels for the plurality of control subjects; and
   assigning a relative expression value for each fraction, wherein the relative expression value is 1 to n for each corresponding fraction from highest to lowest expression level;
c. comparing the expression level of each gene of the immune signature of the subject to the expression levels of each gene of the immune signature of the plurality of control subjects;
d. assigning a relative expression value to each gene of the immune signature of the subject based on the expression value assigned to the corresponding gene of the immune signature of the plurality of control subjects in steps a and b; and
e. calculating the sum of the relative expression values assigned to each gene of immune signature of the subject to provide the immune score.

20. The method of clause 18, wherein calculating the immune score comprises:
calculating the difference between:
   the sum of the expression level of each gene of the immune signature of the subject having the first direction of association; and
   the sum of the expression level of each gene of the signature of the subject having the second direction of association.

21. The method of clause 18, wherein calculating the immune score comprises:
calculating a ratio of expression level of genes of the immune signature of the subject having the first direction of association to;
the genes of the immune signature of the subject having the second direction of association.

22. The method of any of clauses 18 to 21, wherein the disease is cancer.

23. The method of clause 22, wherein:
the cancer is breast cancer or endometrial cancer and the immune associated genes comprises one or more of CCL5, CD3D, CXCL9, CXCL10, GBP1, GBP4, GBP5, GZMB, IDO1.

24. The method of any of clauses 1 to 16, further comprising calculating an immune score according to any of clauses 17 to 23 and wherein the immune score is used to further stratify the subject into one or more immune risk subsets (Tier 3 groups).

25. The method of any of clauses 1 to 24, wherein the method further comprises further stratifying the subject into an intermediate or high risk subset (Tier 4 groups) based on a median risk score.

26. The method of clause 25, wherein the median score comprises the median value of the risk score calculated for one or more of each risk group, each risk subgroup and/or each immune risk subset, for the plurality of control subjects; and wherein a risk score for the subject greater than the median score is indicative of a first prognosis and a score less than the median score is indicative of a second prognosis.

27. The method of any of clauses 1 to 26, wherein the method further comprises stratifying the subject based on the mutational status of one or more additional Group A and/or Group B genomic aberrations to provide sub-risk groups (Tier 5 groups).

28. The method of any of clauses 5 to 16 and clauses 24 to 27 wherein the method further comprises analysing one or more of each risk subgroup, each risk subset, each immune risk subset and/or each sub-risk group by Gene Set Enrichment Analysis (GSEA), over-representation analysis, pathway/network enrichment analysis, proteomic analysis, metabolomic analysis, epigenetic analysis, methylation analysis, and/or acetylation analysis.

29. The method of any of clauses 2 to 28, wherein prognosis comprises any one or more of, likelihood of relapse, time to relapse, overall survival, disease-free survival, recurrence-free survival, metastasis-free survival, event-free survival, time to metastasis, likelihood of metastasis, efficacy of a treatment and/or percentage survival rate over a period of time.

30. A method of determining a prognosis for a subject suffering from a disease, the disease comprising one or more genomic aberrations, the method comprising:
a. providing a subject sample;
b. identifying one or more genomic aberrations associated with the disease from the subject sample;
c. classifying the one or more genomic aberrations according to clause 1 and stratifying the subject according to any one of clauses 2 to 29; and
d. determining a prognosis for the subject based on the risk score and/or the immune risk score;
   wherein prognosis comprises any one or more of, likelihood of relapse, time to relapse, overall survival, disease-free survival, recurrence-free survival, metastasis-free survival, event-free survival, time to metastasis, likelihood of metastasis, efficacy of a treatment, underlying disease biology and/or percentage survival rate over a period of time.

31. The method of clause 30 wherein the method comprises determining a treatment based on the prognosis.

32. The method of clause 31, wherein the method further comprises providing the treatment to the subject.

33. The method of clauses 30 or 32, wherein the treatment is selected from one or more: chemotherapy, hormone therapy, radiotherapy, immunotherapy, targeted therapy, surgery and/or providing no therapeutic intervention.

34. A treatment for cancer for use in a method of treating a subject suffering from a cancer comprising one or more genomic aberrations, wherein the subject has been stratified according to any of clauses 2 to 29; The method of any preceding wherein the disease is metastatic cancer the method comprises stratifying the primary and metastatic tumour specimens according to any of clauses 2 to 29.

35. The method of any of clauses 1 to 29, wherein the DEGs identified as associated with the one or more genomic aberrations are used to determine a treatment plan and/or provide a prognosis for a subject suffering from a further disease comprising the one or more genomic aberrations.

36. Use of the method according to any one of clauses 1 to 29, as a companion diagnostic.

37. The method of any of clauses 1 to 29, wherein the method is a computer implemented method.

38. A computer program comprising instructions which, when the program is executed by a computer, cause the computer to carry out the method according to any of clauses 1 to 29 and 37.

39. A computer-readable storage medium comprising instructions which, when executed by a computer, cause the computer to carry the method according to any of clauses 1 to 29 and 37.

40. An apparatus comprising the computer-readable storage medium according to clause 39, for use as a companion diagnostic.

41. A computer-implemented method for generating a classification model for classifying genomic aberrations to stratify patients into one or more groups, the method comprising:
a. identifying genes in a plurality of control subjects suffering from the disease that undergo a change of expression in response to a first genomic aberration and selecting the genes that undergo a change of expression in response to the first genomic aberration to provide a first set of differentially expressed genes (DEGs) associated with the first genomic aberration;
b. identifying DEGs of the first set of DEGs that overlap with DEGs of a control set of DEGs for a control genomic aberration and selecting the overlapping DEGs to form a first set of overlapping DEGs;
c. comparing the fold direction of change of expression of each DEG of the first set of overlapping DEGs to a fold direction change of expression of the corresponding DEG of the control set of DEGs;
d. classifying the first genomic aberration into a first or second group wherein:
   i. the first group comprises at least 51% overlapping DEGs that comprises a fold direction of change of expression that is the same as the fold direction of change of expression as the corresponding DEG of the control genomic aberration (Group A);
   ii. the second group comprises at least 51% overlapping DEGs that comprise a fold direction of change of expression that is inverse to the fold direction of change of expression of the corresponding DEG of the control genomic aberration (Group B);
e. generating a model comprising at least one classified genomic aberration.

42. The method of clause 41, wherein the method comprises stratifying a subject suffering from the disease, the method further comprising;
calculating a risk score for the subject based on the classified genomic aberration and the subject is stratified based on the risk score; and wherein the calculated risk score is indicative of prognosis of the subject.

43. The method of clause 42, wherein the method further comprises a method according to any one of clauses 3 to 29.

44. The method according any preceding clause, wherein the method comprises determining one or more group-specific biomarkers for each of a or the Tier 1, Tier 2, Tier 3, Tier 4 and/or Tier 5 groups, wherein the group-specific biomarkers are determined by one or more omics-based methods, such as proteomic analysis, epigenetic analysis, and/or metabolomic analysis.

45. A method for predicting a prognosis of a subject suffering from a disease comprising;
a. determining one or more group-specific biomarkers according to clause 44;
b. measuring a level of one or more of the group-specific biomarkers in a sample obtained from the subject;
c. classifying the subject into one or more of the Tier 1, Tier 2, Tier 3, Tier 4 and/or Tier 5 groups based on the level of the one or more of group-specific biomarkers; and
d. predicting the prognosis of the subject based on the classification.

1.-46. (canceled)

47. A method of classifying one or more genomic aberrations associated with a disease, the method comprising:
a. identifying genes in a plurality of control subjects suffering from the disease that undergo a change of expression in response to a first genomic aberration from a plurality of genomic aberrations;
b. selecting the genes that undergo a change of expression in response to the first genomic aberration to provide a first set of differentially expressed genes (DEGs) associated with the first genomic aberration;
c. identifying DEGs of the first set of DEGs that overlap with DEGs of a control set of DEGs for a control genomic aberration and selecting the overlapping DEGs to form a first set of overlapping DEGs;
d. comparing a fold direction of change of expression of each DEG of the first set of overlapping DEGs to a fold direction change of expression of the corresponding DEG of a control set of DEGs;
e. classifying the first genomic aberration into a first or second group wherein:
i. the first group comprises at least 51% overlapping DEGs that comprises the fold direction of change of expression that is the same as the fold direction of change of expression as the corresponding DEG of the control genomic aberration (Group A);
ii. the second group comprises at least 51% overlapping DEGs that comprise a fold direction of change of expression that is inverse to the fold direction of change of expression of the corresponding DEG of the control genomic aberration (Group B).

48. The method of claim 47, wherein the method further comprises:
calculating a risk score for the subject based on the classified genomic aberration; and
stratifying the subject based on the risk score, wherein the risk score is indicative of prognosis of the subject.

49. The method of claim 47, wherein the method further comprises:
a. classifying at least one genomic aberration associated with the disease as Group A and at least one second genomic aberration associated with the disease as Group B;
b. selecting a representative Group A genomic aberration and a representative Group B genomic aberration; wherein each representative genomic aberration is selected based on the frequency of occurrence of the genomic aberration for disease and the number of DEGs associated with the genomic aberration.

50. The method of claim 49, wherein the method further comprises:
a. classifying one or more further genomic aberrations associated with the disease by comparing DEGs associated with the one or more further genomic aberrations to the same DEGs of the first set of overlapping DEGs of the representative Group A genomic aberration and the representative Group B genomic aberration;
b. determining a similarity between the fold direction change of the DEGs;
c. classifying the one or more further genomic aberrations as a Group A or Group B genomic aberration based on the similarity.

51. The method of claim 49, wherein the method further comprises:
a. determining DEGs associated with the representative Group A genomic aberration and/or one or more further Group A genomic aberrations that overlap with DEGs associated with the representative Group B genomic aberration and/or one or more further Group B genomic aberrations and selecting the overlapping DEGs to form a shared set of DEGs;
b. selecting two or more DEGs of the shared set of DEGs to provide one or more disease-specific gene signatures wherein each disease-specific gene signature comprises at least one DEG that has a direction of fold change of expression that is inverse between the representative Group A and representative Group B genomic aberrations;
c. providing an expression level for each DEG of each disease-specific gene signature based on a level of RNA transcript for each DEG for the plurality of control subjects;
d. providing an expression level for each DEG of the disease-specific gene signature for the subject;
e. calculating the risk score for the subject based on the fold direction change of expression and/or the expression level of each DEG of each disease-specific gene signature for the plurality of control subjects;
wherein the subject is stratified as high risk or a low risk based on the calculated risk score (Tier 1 groups).

52. The method of claim 51, wherein calculating the risk score comprises:
a. sorting the expression level of each DEG of the disease-specific gene signature of the plurality of control subjects that is upregulated for the corresponding DEG of the representative Group A genomic aberration in ascending order of the expression level; and dividing the sorted expression levels into 1 to n fractions based on a dynamic range of the expression levels for the plurality of control subjects; and assigning a relative expression value for each fraction, wherein the relative expression value is 1 to n for each corresponding fraction from lowest to highest expression level;
b. sorting the expression level of each DEG of the disease-specific gene signature of the plurality of control subjects that is downregulated for the corresponding DEG of the representative Group A genomic aberration in ascending order of the expression level; and dividing the sorted expression levels into 1 to n fractions based on a dynamic range of the expression levels for the plurality of control subjects; and assigning a relative expression value for each fraction, wherein the relative expression value is 1 to n for each corresponding fraction from highest to lowest expression level;
c. comparing the expression level of each DEG of the disease-specific gene signature of the subject to the expression levels of each DEG of the disease-specific gene signature of the plurality of control subjects;
d. assigning a relative expression value to each DEG of the disease-specific gene signature of the subject based on the expression value assigned to the corresponding DEG of the disease-specific gene signature of the plurality of control subjects in steps a and b;
e. calculating the sum of the relative expression values assigned to each DEG of the disease-specific gene signature of the subject to provide the risk score.

53. The method of claim 51, wherein calculating the risk score comprises:
a. calculating the difference between
(i) the sum of the expression level of each DEG of the disease-specific gene signature of the subject that upregulated for the corresponding DEG of the representative Group A genomic aberration, one or more further Group A genomic aberrations and/or control genomic aberration;
(ii) the sum of the expression level of each DEG of the disease-specific gene signature of the subject that is downregulated for the corresponding DEG of the representative Group A genomic aberration, one or more further Group A genomic aberrations and/or control genomic aberration.

54. The method of claim 51, wherein calculating the risk score comprises, calculating weights from the effect of each gene included in the disease-specific gene signature on the clinical outcome based on the coefficient value of each gene using the following formula: Score=Σni=1 Expi*βi, where Expi is the expression value of each gene, and βi is the regression coefficient of a multivariate Cox analysis for each gene that makes up the disease-specific gene signature.

55. The method of claim 51, wherein calculating the risk score comprises, calculating a ratio of expression levels for each DEG of the disease-specific gene signature of the subject that is upregulated for the corresponding DEG of the representative Group A genomic aberration, one or more further Group A genomic aberrations and/or control genomic aberration to the expression levels for each DEG of the disease-specific gene signature of the subject that is downregulated for the corresponding DEG of the representative Group A genomic aberration, one or more further Group A genomic aberrations and/or control genomic aberration.

56. The method of claim 51, wherein selecting two or more DEGs of the shared set of DEGs to provide the one or more disease-specific gene signatures comprises selecting at least two DEGs from the shared set of DEGs having a statistical significance lower than a threshold value.

57. The method of claim 51, further comprising determining efficiency of the prognostic disease-specific gene signature model by assessing based on statistical parameters.

58. The method of claim 57, wherein the statistical parameters are selected from one or more of: area under the curve (AUC) of receiver operating characteristic (ROC) curve, C-Index, Youden's index at 100% sensitivity (sensitivity+specificity−1), and/or reversed model size (1−nᵢ,/n, where nᵢ, is the number of genes in a defined disease-specific gene signature model and n is the total number of prognostic disease-specific gene signature models).

59. The method of claim 47, wherein the control genomic aberration is selected from the most frequently occurring genomic aberrations for the disease.

60. The method of claim 47, wherein the control genomic aberration comprises at least one TP53 gene mutation and wherein
a. the disease is breast cancer: the representative Group A genomic aberration comprises TP53 gene mutations, and the representative Group B genomic aberration comprises PIK3CA gene mutations; or
b. the disease is prostate cancer: the representative Group A genomic aberration comprises TP53 gene mutations, and the representative Group B genomic aberration comprises SPOP gene mutations; or
c. the disease is endometrial cancer, and the representative Group A genomic aberration comprises TP53 gene mutations, and the representative Group B genomic aberration comprises PTEN gene mutations.

61. The method of claim 51, wherein the method further comprises stratifying the subject into one or more risk subgroups (Tier 2 groups) based on the mutational status of the representative Group A and/or Group B genomic aberrations, wherein the one or more risk subgroups (Tier 2 groups) comprise a further indicator of prognosis.

62. The method of claim 51, wherein the method further comprises stratifying the subject into sub-risk groups (Tier 5 groups) based on the mutational status of one or more additional Group A and/or Group B genomic aberrations.

63. The method of claim 61, wherein
a. the disease is breast cancer and wherein the risk subgroup is selected from: i. TP53-WT and PIK3CA-WT; ii. TP53-WT and PIK3CA-MUT; iii. TP53-MUT and PIK3CA-WT; or iv. TP53-MUT and PIK3CA-MUT; or
b. the disease is prostate cancer and wherein the risk subgroup is selected from: i. TP53-WT and SPOP-WT; ii. TP53-WT and SPOP-MUT; iii. TP53-MUT and SPOP-WT; or iv. TP53-MUT and SPOP-MUT; or
c. the disease is endometrial cancer and wherein the risk subgroup is selected from: i. TP53-WT and PTEN-WT; ii. TP53-WT and PTEN-MUT; iii. TP53-MUT and PTEN-WT; or iv. TP53-MUT and PTEN-MUT.

64. The method of claim 61, wherein the one or more risk subgroups (Tier 2 groups) comprises mutational status of one or more further Group A and/or Group B genomic aberrations.

65. The method of claim 48, further comprising calculating an immune risk score for a subject suffering from a disease, the disease comprising one or more genomic aberrations, by:
a. selecting two or more immune associated genes associated with the disease to form at least one immune signature;
b. optionally performing statistical analysis such as Lasso, univariate, and/or multivariate Cox regression analyses to identify prognosis-related immune associated genes for a disease-specific immune signature;
c. sorting the expression level of each immune associated gene of the immune signature for the plurality of control subjects having a first direction or a second direction of association of expression in ascending order of the expression level;
d. dividing the sorted expression levels into 1 to n fractions based on a dynamic range of the expression levels for the plurality of control subjects;
e. assigning a relative expression value for each fraction, wherein the relative expression value is 1 to n for each corresponding fraction, from lowest to highest expression level or highest to lowest expression level, for the first direction and the second direction respectively;
f. comparing the expression level of each gene of the immune signature of the subject to the expression levels of each gene of the immune signature of the plurality of control subjects;
g. assigning a relative expression value to each gene of the immune signature of the subject based on the expression value assigned to the corresponding gene of the immune signature of the plurality of control subjects in steps a and b; and
h. calculating the sum of the relative expression values assigned to each gene of immune signature of the subject to provide the immune risk score, wherein the immune risk score is used to further stratify the subject into one or more immune risk subsets (Tier 3 groups).

66. The method of claim 65, wherein the method further comprises stratifying the subject wherein stratifying comprises,
a. calculating an immune risk score based on the direction of association and the expression level of the each gene of immune signature of the subject
b. stratifying the subject as high immune risk, a low immune risk and/or an intermediate immune risk based on the calculated immune risk score, wherein the immune risk score is indicative of a prognosis of the subject
c. determining the efficiency of the prognostic immune signature model by assessing based on statistical parameters, wherein the statistical parameters are selected from one or more of: area under the curve (AUC) of receiver operating characteristic (ROC) curve, C-Index, Youden's index at 100% sensitivity (sensitivity+specificity−1), and/or reversed model size (1−nᵢ/n, where nᵢ is the number of genes in a defined disease-specific immune signature model and n is the total number of prognostic immune signature models).

67. The method of claim 66, wherein calculating the immune risk score comprises calculating the difference between the sum of the expression level of each gene of the immune signature of the subject having the first direction of association and the sum of the expression level of each gene of the signature of the subject having the second direction of association.

68. The method of claim 66, wherein calculating the immune risk score comprises calculating weights from the effect of each gene included in the immune signature on the clinical outcome based on the coefficient value of each gene using the following formula: Score=Σᵢ₌₁ Expi*βi, where Expi is the expression value of each gene, and βi is the regression coefficient of a multivariate Cox analysis for each gene that makes up the immune signature.

69. The method of claim 66, wherein calculating the immune risk score comprises calculating a ratio of expression level of genes of the immune signature of the subject having the first direction of association to the genes of the immune signature of the subject having the second direction of association.

70. The method of claim 51, wherein the subjects are further stratified into one or more immune subsets (Tier 3 groups) based on an immune risk score.

71. The method of claim 61, wherein the subjects are further stratified into one or more immune subsets (Tier 3 groups) based on an immune risk score.

72. The method of claim 66, wherein the disease is cancer and the immune associated genes comprises one or more of CCL5, CD3D, CXCL9, CXCL10, GBP1, GBP4, GBP5, GZMB, IDO1, NFS1, NKG7, CD247, CD7, CTLA4, CD2, CD38, ICOS, GZMA, GNLY, IL18BP, CD8A, TCRVB, PTPRCAP, CXCR6, SH2D1A, CXCR3, PRF1, PVRIG, ITK, HCST, LTA, PYHIN1, IRF1, MAP4K1, CD3G, PRKCB.

73. The method of claim 48, further comprising calculating a metastatic risk score for a subject suffering from a disease comprising one or more genomic aberration by:
a. selecting two or more metastatic/dissemination associated genes associated with the disease to form at least one disease-specific metastatic signature;
b. assigning a direction of association to each gene of the disease-specific metastatic signature based on a change of expression for a plurality of control subjects;
c. wherein each gene with an increased expression level is designated with a first direction of association and each gene with a decreased expression is designated with a second direction of association inverse to the first direction of association;
d. determining an expression level for each gene of the disease-specific metastatic signature based on a level of RNA transcript for each gene for the plurality of control subjects; and
e. determining an expression level for each gene of the disease-specific metastatic signature based on a level of RNA transcript for each gene for the subject.

74. The method of claim 73, wherein the method further comprises performing statistical analysis such as Lasso, univariate, and/or multivariate Cox regression analyses to identify prognosis-related metastatic/dissemination associated genes for the disease-specific metastatic signature.

75. The method of claim 73, wherein the method further comprises stratifying the subject based on the calculated metastatic risk score, wherein stratifying comprises, calculating a metastatic risk score based on the direction of association and the expression level of each gene of the disease-specific metastatic signature of the subject.

76. The method of claim 75, further comprising stratifying the subject as high metastatic risk, a low metastatic risk or an intermediate metastatic risk based on the calculated metastatic risk score.

77. The method of claim 48, wherein the method further comprises stratifying the subject into an intermediate risk or high risk or low risk subset (Tier 4 groups) based on a median risk score, wherein the median risk score comprises the median value of the risk score calculated for one or more of each risk group, and/or each risk subgroup, for the plurality of control subjects and wherein a score for a subject greater than the median risk score is indicative of a first prognosis and a score less than the median score is indicative of a second prognosis.

78. The method of claim 66, wherein the method further comprises stratifying the subject into an intermediate risk or high risk or low risk subset (Tier 4 groups) based on a median immune risk score, wherein the median immune risk score comprises the median value of the immune risk score calculated for one or more of each immune risk subset, for the plurality of control subjects and wherein a score for a subject greater than the median immune risk score is indicative of a first prognosis and a score less than the median immune risk score is indicative of a second prognosis.

79. The method of claim 75, wherein the method further comprises stratifying the subject into an intermediate risk or high risk or low risk subset (Tier 4 groups) based on a median metastatic risk score, wherein the median metastatic risk score comprises the median value of the risk score calculated for one or more of each metastatic risk subset, for the plurality of control subjects and wherein a score for a subject greater than the median metastatic risk score is indicative of a first prognosis and a score less than the median metastatic risk score is indicative of a second prognosis.

80. The method of claim 75, wherein the metastatic risk score is indicative of a prognosis of the subject.

81. The method of claim 75, wherein the method further comprises determining efficiency of the prognostic disease-specific metastatic signature model by assessing based on statistical parameters, wherein the statistical parameters are selected from one or more of: area under the curve (AUC) of receiver operating characteristic (ROC) curve, C-Index, Youden's index at 100% sensitivity (sensitivity+specificity−1), and/or reversed model size (1−nᵢ/n, where nᵢ is the number of genes in a defined disease-specific metastatic/dissemination signature model and n is the total number of prognostic disease-specific metastatic signature models.

82. The method of claim 75, wherein calculating the metastatic risk score further comprises:
a. sorting the expression level of each gene of the disease-specific metastatic signature for the plurality of control subjects having the first direction or second direction of association of expression in ascending order of the expression level;
b. dividing the sorted expression levels into 1 to n fractions based on a dynamic range of the expression levels for the plurality of control subjects;
c. assigning a relative expression value for each fraction, wherein the relative expression value is 1 to n for each corresponding fraction from lowest to highest or highest to lowest expression level, for the first direction and the second direction respectively;
d. comparing the expression level of each gene of the disease-specific metastatic signature of the subject to the expression levels of each gene of the disease-specific metastatic signature of the plurality of control subjects;
e. assigning a relative expression value to each gene of the disease-specific metastatic signature of the subject based on the expression value assigned to the corresponding gene of the metastatic signature of the plurality of control subjects in steps a and b; and
f. calculating the sum of the relative expression values assigned to each gene of disease specific metastatic signature of the subject to provide the metastatic risk score.

83. The method of claim 82, wherein calculating the metastatic risk score comprises calculating the difference between the sum of the expression level of each gene of the disease-specific metastatic signature of the subject having the first direction of association and the sum of the expression level of each gene of the signature of the subject having the second direction of association.

84. The method of claim 82, wherein calculating the metastatic risk score comprises calculating weights from the effect of each gene included in the disease-specific metastatic signature on the clinical outcome based on the coefficient value of each gene through the following formula: Score=Σⁿᵢ₌₁ Expi*βi, where Expi is the expression value of each gene, and βi is the regression coefficient of a multivariate Cox analysis for each gene that makes up the disease-specific metastatic signature.

85. The method of claim 75, wherein the disease is ER+ HER2− breast cancer and the metastatic associated genes comprises one or more of SEMA3B, DRC3, SPATA4, GREM1, SLC7A2, CELSR2, MCU, FLNB, TBC1D31, DIRAS3, RGS22, CR593862, ENC1, TMEM26, MYL5, RGS4, EIF2B2, NXNL2, CHDH, AGO4, CPT1A, CSPG4, NBPF22P, SLC15A2, RPGR, DDIT4, MYCBPAP, LCN12, DCAKD, DBNDD2, TMEM101, CAPN8 GATAD1, CASP3, AAR2, F8.

86. The method of claim 51, wherein the subjects are further stratified into one or more metastatic risk subsets (Tier 6 groups) based on a metastatic risk score.

87. The method of claim 61, wherein the subjects are further stratified into one or more metastatic risk subsets (Tier 6 groups) based on a metastatic risk score.

88. The method of claim 48, further comprising determining a prognosis of a subject suffering from a disease the method further comprising:
a. providing a subject sample;
b. identifying one or more genomic aberrations associated with the disease from the subject sample;
c. classifying the one or more genomic aberrations;
d. stratifying the subject; and
c. determining a prognosis for the subject based on the risk score.

89. The method of claim 88, wherein the risk score comprises one of a calculated risk score, an immune risk score, a metastatic risk score.

90. The method of claim 89, further comprising determining a prognosis for the subject based on one of: a risk score, a risk score and an immune risk score, a risk score and a metastatic risk score, a risk score, an immune risk score and/or a metastatic risk score, mutational status of the representative Group A and/or Group B genomic aberrations, a risk score and mutational status of the representative Group A and/or Group B genomic aberrations, a risk score, mutational status of the representative Group A and/or Group B genomic aberrations and/or an immune risk score, a risk score, mutational status of the representative Group A and/or Group B genomic aberrations, an immune risk score and/or a metastatic risk score, mutational status of one or more additional Group A and/or Group B genomic aberrations, a risk score and mutational status of one or more additional Group A and/or Group B genomic aberrations, a risk score, mutational status of one or more additional Group A and/or Group B genomic aberrations and/or an immune risk score, a risk score, mutational status of one or more additional Group A and/or Group B genomic aberrations, an immune risk score and/or a metastatic risk score.

91. The method of claim 89, wherein determining the prognosis comprises determining any one or more of, likelihood of relapse, time to relapse, overall survival, disease-free survival, recurrence-free survival, metastasis-free survival, event-free survival, time to metastasis, likelihood of metastasis, efficacy of a treatment, identifying underlying disease biology and/or percentage survival rate over a period of time.

92. The method of claim 91, wherein the method further comprises determining a treatment based on the prognosis, wherein the treatment is selected from one or more of: chemotherapy, hormone therapy, radiotherapy, immunotherapy, targeted therapy, surgery and/or providing no therapeutic intervention.

93. The method of claim 92, wherein the treatment based on prognosis is for treatment of cancer.

94. A computer readable medium having computer executable instructions that when executed by a processor of a computing system causes the computing system to carry out the method steps according claim 47.

95. A computer readable medium having computer executable instructions that when executed by a processor of a computing system causes the computing system to execute the steps of:
a. generating a classification model for classifying genomic aberrations;
b. stratify patients into one or more groups;
c. identify genes in a plurality of control subjects suffering from the disease that undergo a change of expression in response to a first genomic aberration and selecting the genes that undergo a change of expression in response to the first genomic aberration to provide a first set of differentially expressed genes (DEGs) associated with the first genomic aberration;
d. identify DEGs of the first set of DEGs that overlap with DEGs of a control set of DEGs for a control genomic aberration and selecting the overlapping DEGs to form a first set of overlapping DEGs;
e. compare the fold direction of change of expression of each DEG of the first set of overlapping DEGs to a fold direction change of expression of the corresponding DEG of the control set of DEGs;
f. classify the first genomic aberration into a first or second group wherein:
i. the first group comprises at least 51% overlapping DEGs that comprises a fold direction of change of expression that is the same as the fold direction of change of expression as the corresponding DEG of the control genomic aberration (Group A);
ii. the second group comprises at least 51% overlapping DEGs that comprise a fold direction of change of expression that is inverse to the fold direction of change of expression of the corresponding DEG of the control genomic aberration (Group B);
g. generate a model comprising at least one classified genomic aberration;
h. calculate a risk score for the subject based on the classified genomic aberration, wherein the calculated risk score is indicative of prognosis of the subject; and
i. stratify the subject based on the risk score.
j. determine one or more group-specific biomarkers for each of a Tier 1, a Tier 2, a Tier 3, a Tier 4, a Tier 5 and/or Tier 6 groups, wherein the group-specific biomarkers are determined by one or more omics-based methods, such as proteomic analysis, transcriptomic analysis, Gene Set Enrichment Analysis (GSEA), Gene Set Variation Analysis (GSVA), over-representation analysis, pathway/network enrichment analysis, single sample Gene Set Enrichment Analysis (ssGSEA), epigenetic analysis, and/or metabolomic analysis; and
k. predict a prognosis of a subject suffering from a disease comprising:
i. determining one or more group-specific biomarkers;
ii. measuring a level of one or more of the group-specific biomarkers in a sample obtained from the subject;
iii. classifying the subject into one or more of the Tier 1, Tier 2, Tier 3, Tier 4, Tier 5 and/or Tier 6 groups based on the level of the one or more of group-specific biomarkers; and
iv. predicting the prognosis of the subject based on the classification.

96. The computer readable medium of claim 95, wherein the steps of the computer readable medium is used for a diagnostic, a companion diagnostic and/or a prognostic test device.
